SEQUENCE LISTING (14 sequences)

SEQ ID NO: 1 - protein, 7 aa, Artificial Sequence
  Description of Artificial Sequence Synthetic peptide
RDVLPGT

SEQ ID NO: 2 - protein, 7 aa, Artificial Sequence
  Description of Artificial Sequence Synthetic peptide
RDVVPGG

SEQ ID NO: 3 - protein, 16 aa, Drosophila melanogaster
RQIKIWFQNRRMKWKK

SEQ ID NO: 4 - protein, 7 aa, Artificial Sequence
  Description of Artificial Sequence Synthetic peptide
PTDLVRG

SEQ ID NO: 5 - protein, 4 aa, Artificial Sequence
  Description of Artificial Sequence Synthetic peptide
LPGT

SEQ ID NO: 6 - protein, 7 aa, Artificial Sequence
  Description of Artificial Sequence Synthetic peptide
TGPLVDR

SEQ ID NO: 7 - protein, 4 aa, Artificial Sequence
  Description of Artificial Sequence Synthetic peptide
RDVL

SEQ ID NO: 8 - protein, 6 aa, Artificial Sequence
  Description of Artificial Sequence Synthetic peptide
RDVLPG

SEQ ID NO: 9 - protein, 7 aa, Artificial Sequence
  Description of Artificial Sequence Synthetic peptide
RNVCPGC

SEQ ID NO: 10 - protein, 6 aa, Artificial Sequence
  Description of Artificial Sequence Synthetic peptide
RNVLPG

SEQ ID NO: 11 - protein, 6 aa, Artificial Sequence
  Description of Artificial Sequence Synthetic peptide
RDVVPG

SEQ ID NO: 12 - protein, 6 aa, Artificial Sequence
  Description of Artificial Sequence Synthetic peptide
DVVPGG

SEQ ID NO: 13 - protein, 4 aa, Artificial Sequence
  Description of Artificial Sequence Synthetic peptide
VVDR

SEQ ID NO: 14 - DNA, 10 bp, Artificial Sequence
  Description of Artificial Sequence Synthetic oligonucleotide
gggactttcc

(19) United States
(12) Patent Application Publication
(10) Pub. No.: US 2008/0064643 A1
(43) Pub. Date: Mar. 13, 2008

(54) Myd88 Homodimerization Inhibitors

(76) Inventors: Paolo Carminati, Pomezia (IT); Grazia Gallo, Pomezia (IT); Nicola Fanto, Pomezia (IT); Vito Ruggiero, Pomezia (IT); Marica Sassano, Piana Di Monte Verna (IT); Domenico Mastroianni, Piana Di Monte Verna (IT)

Correspondence Address: NIXON & VANDERHYE, PC, 901 NORTH GLEBE ROAD, 11TH FLOOR, ARLINGTON, VA, 22203, US

(21) Appl. No.: 11/793,516

(22) PCT Filed: Dec. 16, 2005
(86) PCT No.: PCT/EP05/56847

§ 371 Date: Jul. 20, 2007

(30) Foreign Application Priority Data
Dec. 20, 2004 (EP) 04425929.9

(51) Int. Cl. A61K 38/04; A61P 43/00; C07K 7/02

(52) U.S. Cl. 514/16; 530/323

(57) ABSTRACT

The present invention relates to peptidic and peptidomimetic compounds with the formula
(X⁻)AA₁-AA₂-AA₃-AA₄-AA₅-AA₆-AA₇ in which the various groups are defined in the description here below, which mimic a particular protein portion of MyD88, preventing its homodimerisation and interfering with its interaction with the TIR domain. The present invention also provides procedures for the preparation of said compounds, pharmaceutical compositions containing them and their use as medicaments, particularly for the treatment of inflammatory and autoimmune diseases.

The present invention relates to peptidic and peptidomimetic compounds that mimic a particular protein portion of MyD88, preventing its homodimerisation and interfering with its interaction with the TIR domain.

The present invention provides processes for the preparation of said compounds, pharmaceutical compositions containing them and their use as medicaments, particularly for the treatment of inflammatory and autoimmune diseases.

### BACKGROUND TO THE INVENTION

The inflammatory response is usually a reaction of a defensive nature, activated by living organisms in order to demarcate and subsequently eradicate the damage resulting from physicochemical insults and infectious attack. In some cases, however, acute inflammatory events, due to persistence of the stimulus, evolve into states of inflammatory hyperactivation, which tend to become chronic, often resulting in the self-destruction of the normal surrounding tissues. This process is due to the increased induction of adhesive molecules, transmigration of inflammatory cellular elements in the site of the pathogenic insult and the consequent release of a battery of inflammatory mediators (Shanley, T. P., et al.; Mol. Med. Today, 40-45, 1995).

The transcription sequence and possible production of the various mediators is under the control of particular protein factors known as transcriptional factors or TFs (Müller, C. W.; Curr. Opin. Struct. Biol., 11:26-32, 2001). These factors, once activated, bind to specific consensus regions present in the DNA and act as molecular switches for the induction or overregulation of the gene expression of inflammatory agents.

The most frequently studied transcriptional factor, on account of its involvement in various inflammatory conditions, is undoubtedly the factor NF-κB, which recognises consensus sequences for the enhancers of various genes coding for pro-inflammatory cytokines (TNF, IL-1, IL-2, IL-6, IL-11, IL-17, GM-CSF), chemokines (IL-8, RANTES, MIP-1α, MCP-2), adhesive molecules (ICAM-1, VCAM-1, E-selectin) and enzymes producing inflammatory mediators (iNOS and COX2) (Ghosh, S., et al.; Annu. Rev. Immunol., 16: 225-260, 1998).

In response to damaging stimuli of various kinds, NF-κB activation is observed in almost all the cells involved in the immune response: neutrophils, macrophages, lymphocytes and endothelial, epithelial and mesenchymal cells. The immediate, transitory activation of NF-κB therefore constitutes a characteristic of primary importance in the functioning of the normal physiological response to pathogenic damage. Nevertheless, the dysregulation of this fine mechanism that manifests itself in the form of excessive, persistent activation has been found to be closely associated with chronic inflammatory diseases (Barnes, P. J. and Karin, M.; New Engl. J. Med., 336: 1066-1071, 1997).

The crucial role played by NF-κB in causing chronic inflammatory diseases makes this factor a therapeutic target of choice for targeted therapeutic interventions.

In those cases in which the safety of an “anti-NF-κB” therapy is considered, a distinction must be made between non-specific side effects and effects directly attributable to the intrinsic suppression of NF-κB. Since the latter constitutes a critical point of convergence of a multitude of signals in the context of the normal cellular physiological response, it is plausible to predict that the prolonged generalised and systemic inhibition of NF-κB may give rise to unwanted and damaging effects. For the purposes of the therapeutic application of such antagonists there appears, then, to be an evident need to identify substances that allow effective treatment while at the same time minimising as far as possible the unwanted effects. A parameter of capital importance in designing potential NF-κB antagonists therefore consists in selectivity of action.

It is expected, moreover, that pharmacological agents capable of inhibiting NF-κB, by interfering with the proximal inflammatory signalling system, may be safer than others that act on more distal biochemical events.

The molecular events that result in the binding of IL-1 and that lead to the activation of transcriptional factors such as NF-κB and AP-1 occur via an amplification cascade based on the sequential activation of multiple protein factors.

In particular, the binding of IL-1 induces the formation of the receptor heterocomplex IL-1R/1L-1RacP which subsequently recruits the adaptor protein MyD88. It is worth stressing that the intracytoplasmic domains of IL-1R e IL-1RAcP interact with MyD88 through interactions of a homophilic type that set in between the respective TIR domains. Whereas the carboxyterminal portion of MyD88 (TIR domain) is responsible for the docking of MyD88 to the receptor heterocomplex, the aminoterminal portion (death domain), by interacting with the death domain of the kinase IRAK (in this case, too, through interactions of a homophilic type), allows the recruitment of the latter to the heterocomplex, which is the site where its phosphorylation takes place. After the phosphorylation has taken place, IRAK is believed to detach itself from the complex and interact with the adaptor protein TRAF6 before being degraded in the proteosome. TRAF6, in turn, leads to the activation of the kinase TAK1 which autophosphorylates and subsequently activates the kinases MAP2K and NIK (NF-κB Inducing Kinase). The end result is that MAP2K and NIK lead to the activation of the transcriptional factors AP1 and NF-κB, respectively, involved in the transcription of genes coding for important inflammatory mediators.

Research conducted in recent years has lent support to the hypothesis concerning the existence of a common transduction mechanism in the context of the IL-1R/TLR superfamily. Effectively speaking, it has been demonstrated (O'Neill, L A J and Dinarello, C A; Immunology Today, 21(5):206-209, 2000) that the transduction mechanism of the signal activated by IL-1 is also operative in the signalling of IL-18 ed LPS.

In particular, it has been shown that the adaptor protein MyD88 plays a crucial role in the transduction events triggered by IL-1, IL-18 and LPS. It has been reported, in fact, that MyD88 KO mice, though proving perfectly viable, lack the normal ability to respond to stimulation with LPS (Kawai, T., et al.; Immunity, 11: 115-122, 1999).

These results were confirmed in a different experimental setting in which it was shown that the F56N point mutation of MyD88, which prevents the formation of the functionally active dimer of MyD88, does not induce activation of the NF-κB transcriptional factor (Burns K et al., J Biol Chem 273(20): 12203-12209, 1998).

Further studies (Adachi, O., et al.; Immunity, 9: 143-150, 1998) have established, moreover, that MyD88 KO mice are not responsive to stimulation with IL-1 or IL-18. It has been observed, in fact, that the thymocytes and spleen cells of these KO mice do not activate the normal proliferative response if stimulated with IL-1. In addition, the production of IFN-γ and NK cell activity are not increased as a result of stimulation with IL-18, unlike what happens in wild-type mice.

On the basis of the findings outlined here, it is clear that MyD88 plays a key role in the activation of NF-κB triggered by various different inflammatory stimuli such as IL-1, IL-18, agonists of the IL-1R family and agonists the TLR family, such as, for example, LPS (Takeuchi, O. and Akira, S.; Curr. Top. Microbiol. Immunol., 270:155-67, 2002).

The aim of approaches currently used to antagonise the signalling of inflammatory cytokines has so far been to neutralise specifically the activity of each of them, by resorting to the use of specific monoclonal antibodies, receptor antagonists or soluble receptors. The obvious purpose was to selectively prevent, from the outside, the binding of the cytokines to the relevant membrane receptors.

The most recent literature suggests, however, that the adoption of experimental approaches based on the inhibition of intracellular signalling mediated by cytoplasmic adaptor proteins may be an effective innovative strategy (L. A. J. O'Neill and C. A. Dinarello; Immuno. Today, 21:206-209, 2000; M. Muzio, et al.; J. Leukoc. Biol., 67:450-456, 2000; J. M. Schuster and P. S, Nelson; J. Leukoc. Biol., 67:767.773, 2000).

Therefore, the inhibition of an adaptor protein such as MyD88 which is involved in the activation of NF-κB, triggered by signals from receptors present on the outer surface of the ell membrane which recognise distinct ligands but which share the same transduction pathway, in principle proves more effective than inhibition of the individual ligand activities.

On alignment of the primary sequences of the TIR domains of the receptors IL-1R/TLR and MyD88, one of the zones conserved consists in a loop between the second beta strand and the second alpha-helix (BB loop) whose consensus sequence is RDXΦ₁Φ₂GX where X is any amino acid and Φ₁Φ₂ are two hydrophobic; in particular, Φ₂ is a proline, except in IL-1RI where it is valine. It is known that the mutations R677->E P681->H and G682->V in TLR4 abolish the transmission of the signalling; moreover, on comparing the crystallographic coordinates of the intracytoplasmic TIR domain of the receptor TLR2 and TLR2 mutated to P681->H, there are no structural variations affecting the loop or the zones adjacent to it (Nature, 2000, Vol. 408, 111). This zone may therefore be involved as an adaptor protein/receptor interaction interface. It is assumed therefore that this zone is also important for the homo-dimerisation of MyD88.

Also known in this connection is a study conducted by Rebek et al. (Bartfai, T., et al.; Proc. Natl. Acad. Sci. USA, 100:7971, 2003) who demonstrated the potential applicability of peptidomimetic compounds capable of interfering with the MyD88/IL1-RI interaction at the level of the intracellular TIR domain; the mimetic of the central portion of the BB loop, having (F/Y)-(V/L/I)-(P/G) as its consensus sequence in different toll receptors and homologues of MyD88, is capable of inhibiting in vitro the phosphorylation of protein kinase p38 in the EL4 cell line stimulated with IL-1β, but also of significantly attenuating in vivo the febrile response in mice injected with murine recombinant IL-1β. This compound, however, is not effective in inhibiting the MyD88/TLR4 interaction.

Experts in the field would welcome a compound that inhibits not the single MyD88/receptor interaction, but the homodimerisation of the adaptor protein, which may thus result in the inhibition of a larger number of pro-inflammatory signals and thus prove therapeutically more effective.

Additionally, a recent invention patented by Yale University, CT, USA (WO 02/090520 A2) claims the use of TIRAP polypeptides for antagonizing MyD88-independent signalling in response to TLR4 ligation.

TIRAP is a novel protein containing a Toll/IL-1 receptor (TIR) domain independently identified by Medzhitov (Horng, T., Barton, G. M., and Medzhitov, R.; Nat. Immunol. 2:835-841, 2001) and by O'Neill (Fitzgerald, K A., Palsson-McDermott, E. M. et al.; Nature 413:78-83, 2001). Despite initial expectations that TIRAP might participate in MyD88-independent NF-kB activation, however, subsequent studies have revealed that TIRAP is not involved in the MyD88-independent pathway, but rather acts an adapter in the MyD88-dependent signaling pathways initiated via TLR2 and TLR4 (Yamamoto, M., Sato, S., Hemmi, H.; Nature 420:324-329, 2002). Actually, the same investigators discovered that an additional adaptor, named Trif, was indeed involved in the MyD88-independent activation of NF-kB (Yamamoto, M., Sato, S., Hemmi, H. et al.; Science 301, 640-643). The presently available evidence suggests that all of the TLRs, with the sole exception of TLR3, utilize MyD88 (Takeda, K and Akira, S.; Int Immunol. 17:1-14, 2005). Dunne et al. carried out an in-depth study into the interaction of TIRAP and MyD88 with TLR2 and TLR4 showing that TIRAP and MyD88 actually bind to different regions of TLR2 and TLR4 (Dunne, A., Ejdeback, M., Ludidi, P. L. et al.; J Biol Chem 278:41443-41451, 2003).

Once again, one of skill in the art would welcome a compound that inhibits neither the single MyD88/receptor interaction nor an adaptor (TIRAP) involved in the signaling of TLR2 and TLR4 solely. Rather, the inhibition of a single bottleneck adapter (MyD88) involved in the transduction of the signalling from all of the known TLRs (except TLR3) is envisioned to antagonise a larger number of pro-inflammatory signals and thus prove therapeutically more efficacious.

On the basis of the information currently available in the literature, it is believed that a number of diseases deriving from dysregulation of the signalling of the TLR/IL-1R receptor system comprise, but are not limited to:
Inflammatory and autoimmune diseases such as, for example, arthritis, gouty arthritis, chronic inflammatory bowel disease (IBD), psoriasis, type 1 diabetes, multiple sclerosis, asthma, and systemic lupus erythematosus (see, for example, Sabroe, I., et al.; J. Immunol.; 171:1630-5, 2003; Liu-Bryan R et al. Arthritis Rheum. 52:2936-46, 2005; Joosten, L A, et al.; J Immunol., 171:6145-53, 2003; Sabroe, I., et al.; Clin. Exp. Allergy, 32:984-9, 2002; Lehnardt, S.; Proc. Natl. Acad. Sci. USA, 100:8514-9, 2003; Choe, J Y, et al.; J. Exp. Med., 197:537-42, 2003; Sabroe, L; Thorax, 59:81, 2004; Bellou, A.; Curr. Opin. Allergy Clin. Immunol., 3:487-94, 2003; O'Neill, L A; Curr. Opin. Pharmacol., 3:396-403, 2003; Schon, M., et al.; Clin. Exp. Immunol., 123:505-10, 2001; Leadbetter, E A et al.; Nature, 416:603-7, 2002; Rifkin, I R et al.; Immunol Rev. 204.27-42, 2005.).
Cardiovascular and atherogenic diseases such as, for example, myocardial infarct, viral myocarditis, atherosclerosis, vein graft atherosclerosis, thrombosis, re-stenosis, re-stenosis due to stents and re-stenosis due to angioplasty (see, for example, de Kleijn, D., and Pasterkamp G.; Cardiovasc Res., 60:58-67, 2003; Oyama, J.-L, et al.; Circulation, 109: 784-789, 2004; Satoh, M., et al.; Lab. Invest., 84:173-81, 2004; Thomas, J A, et al.; Am. J. Physiol. Heart Circ. Physiol., 285:H597-606, 2003); Fairweather, D., et al.; J. Immunol., 170:4731-7, 2003; Kiechl, S., et al.; Ann. Med., 35:164-71, 2003; Edfeldt, K, et al.; Circulation, 105.1158-1161, 2002; Arditi et al., US20030148986).
Sepsis and shock (see, for example, Read, RC., and Wyllie, D H.; Curr. Opin. Crit. Care; 7:371-5, 2001; Carrillo-Esper, R.; Cir. Cir., 71:252-8, 2003; Knuefermann, P.; Chest, 121: 1329-1336, 2002; Knuefermann, P., et al.; Circulation, 106: 2608-2615, 2002).
Transplant rejection (see, for example, Goldstein, D R., et al.; J. Clin. Invest., 111:1571-1578, 2003; Belperio, J. A.; Am. J. Respir. Crit. Care Med., 168: 623-624, 2003).
Cancer (see, for example, Huang, B, et al.; Cancer Res. 65: 5009-14, 2005).
Viral infections (see, for example, Bafica, A. et al.; J Immunol. 172:7229-34, 2004; Equils, O., et al.; J Immunol 170:5159-5164, 2003; Scheller, C. et al.; J Biol Chem 279:21897-21902, 2004; Sundstrom, J. B. et al.; J Immunol 172:4391-4401, 2004)

Patent application US20030148986, filed in the name of Cedars-Sinai Medical Center, describes various ways of inhibiting the expression or the biological activity of the protein MyD88. These also include the use of peptidomimetic agents that prevent the signalling of the protein. This inhibition is accomplished with small peptides (10-20 amino acids) that bind to the TLR-4 receptors, thus preventing the binding with MyD88. Small overlapping segments (approximately 10-20 amino acids) of MyD88 can be separated to test to see which of these prevent the transduction of the MyD88 cell signal by binding to the TLR-4 receptors. After the separation, the segments are duplicated and tested to determine whether the segment comprises at least one portion of MyD88 that binds to the TLR-4 receptor, which will prevent the binding of MyD88 and the transduction of the cell signal. No concrete examples of peptides are given in this reference.

Preliminary studies on the inhibition of the homodimerisation of MyD88 have been conducted by the present inventors using a co-immunoprecipiation assay on a series of natural peptides (synthesised as acetylamide at the N terminal and as primary amide at the carboxyl terminal) comprising the consensus sequence of the BB loop. Table 1 shows the peptides that proved active and the residual interaction found in the MyD88 homodimer as a percentage of the untreated protein. The protein myc-MyD88 is expressed temporarily in HEK293 cells and isolated from cell extracts by immunoprecipitation with the anti-myc antibody. The immunoprecipitated protein is incubated with purified protein GST-MyD88TIR for 60 min at 4° C. in the presence or absence of the synthetic peptide (final concentration 200 μM). After suitable washings, the proteins absorbed to the resin used for the immunoprecipitation are solubilised with SDS and analysed by Western blot for the presence of myc-MyD88 and GST-MyD88TIR. Percentage inhibition of NF-kB activation is also given for these two peptides

TABLE 1

| | | % residual | % NF-kB |
|---|---|---|---|
| Name | Peptide (sequence) | interaction | inhibition |
| ST2348 | MyD88 | 29 | 37 |
| | (Ac-RDVLPGT-NH₂) | | |
| ST2350 | IL-18R | 18 | 20 |
| | (Ac-RDVVPGG-NH₂) | | |

In addition, peptide ST2348, conjugated with a fragment of the Antennapaedia (Ap) protein of Drosophila (Ap-MyD88=ST2345), with the sequence RQIKIWFQNRRMKWKK (penetratin) (Gari, J., and Kawamura, K; TRENDS in Biotechnology, 19:21-28, 2001), proved capable of inhibiting NF-κB activation in HeLa cells stimulated with IL-1α, whereas the corresponding scrambled peptide (ST2403 Ap-PTDLVRG-NH₂) is inactive.

The purpose of the present invention is to identify mimetics of a particular protein portion of MyD88, which prevent the homo-dimerisation of the protein, by interfering with its interaction with the TIR domain. This approach would make it possible to avoid the recruitment of MyD88 to each of the IL-1R/TLR receptors in which it plays its role as an adaptor protein.

The molecules provided by the present invention are useful as medicaments for the therapy of chronic inflammatory diseases and are capable of modulating NF-kB activation mediated by IL-1R/TLR receptors.

The strategy for constructing mimetics of the consensus peptide of the TIR domain of MyD88 is described here below:

The H-Arg-Asp-Val-Leu-Pro-Gly-Thr-OH structure of the consensus peptide of the TIR domain of MyD88 is subdivided into three distinct portions consisting of.

a) a charged portion consisting of the amino acids Arg-Asp,

b) a hydrophobic portion consisting of the amino acids Val-Leu,

c) a β-turn portion consisting of the amino acids Leu-Pro-Gly-Thr.

For each of these portions a certain type of mimetic was chosen to be substituted alternatively or simultaneously in the consensus peptide sequence, maintaining the amide bond as the functional linker group between the three groups:

a) Greater account is taken of the Arg group and it is substituted with an arginino-mimetic group, where what is meant by arginino-mimetic is a chemical structure which when arginine is substituted modulates the basicity of the functional group from the basicity of arginine to zero basicity.

b) Account is taken of the distance between arginine and proline and it is filled with a spacer, where what is meant by spacer is a hydrophobic chemical structure with a limited number of rotational freedom degrees that contains an aromatic linker ring variously substituted and functionalised, only one carboxylic acid group and only one primary amine group engaged in amide bonds.

c) Account is taken of the central portion of the Pro-Gly β-turn and it is substituted with a β-turn mimetic, where what is meant by β-turn mimetic is a chemical structure that, by mimicking the central portion of the Pro-Gly β-turn, allows the molecule to take on a conformation useful for the formation of bonds with the protein MyD88.

### SUMMARY OF THE INVENTION

It has now been found that peptidic and/or peptidomimetic compounds as described here below are capable of mimicking a particular protein portion of MyD88, preventing its homodimerisation and interfering with its interaction with the TIR domain.

Subjects of the present invention are peptidic and/or peptidomimetic compounds with formula (I)
(X−)AA₁-AA₂-AA₃-AA₄-AA₅-AA₆-AA₇ (I)
in which:

X− is an anion of a pharmacologically acceptable acid, or is absent; each of the groups AA₁-AA₇, which may be the same or different, is an amino acid or amino acid mimetic with the following meanings:

AA₁=is the residue of L-arginine (Arg), D-arginine (arg), L-histidine (His), D-histidine (his), or an arginino-mimetic group, where what is meant by arginino-mimetic is a chemical structure that substitutes for arginine and modulates the basicity of the functional group, from the basicity of arginine to zero basicity, with formulas (II), (III) and (IV) 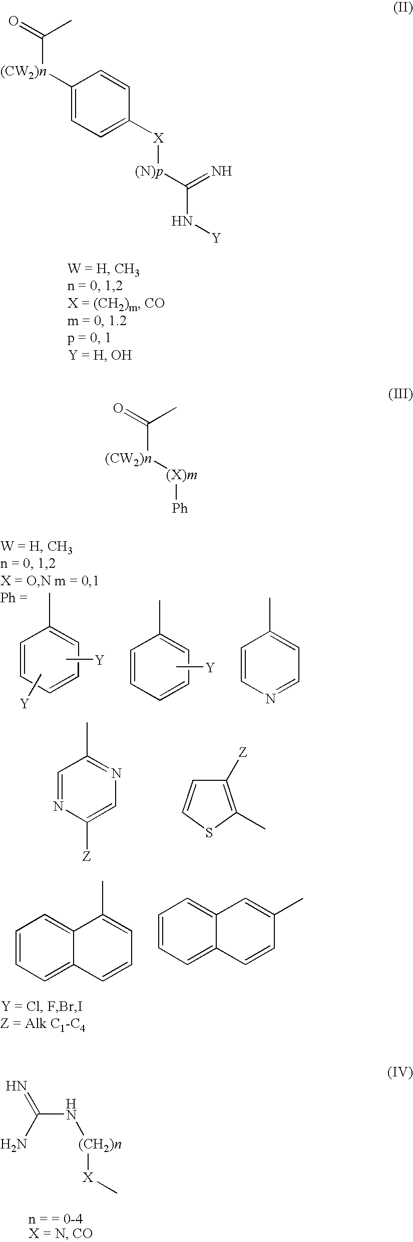
or is absent;

AA₂=L-aspartic acid (Asp), D-aspartic acid (asp), L-asparagine (Asn), D-asparagine (asn), glycine (gly or Gly), or is absent;

AA₃=L-valine (Val), D-valine (val), azavaline (AzaVal), azaglycine (Azagly), azaleucine (AzaLeu);

AA₄=L-leucine, D-leucine, L-valine (Val), D-valine (val), L-cysteine (Cys), D-cysteine (cys), azaleucine (AzaLeu), azavaline (AzaVal)), azaglycine (Azagly);

AA₂-AA₃-AA₄ together can be substituted by a spacer where what is meant by spacer is a hydrophobic chemical structure with a limited number of rotational freedom degrees that contains an aromatic linker ring variously substituted and functionalised, only one carboxylic acid group and only one primary amine group, engaged in amide bonds, with formula (V):

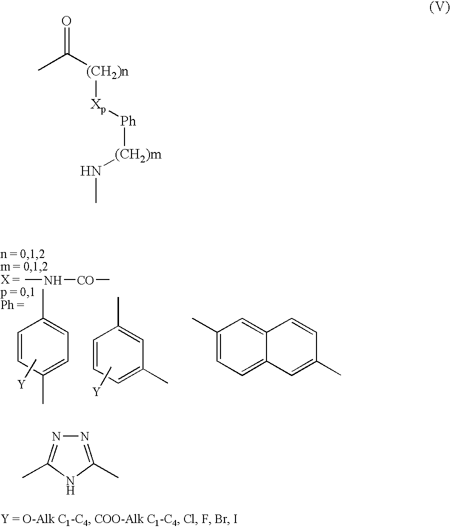

AA₅=L-proline (Pro), D-proline (pro), cis-4,5-(methano)-L-proline (cMe-Pro),

cis-4,5-(methano)-D-proline (cMe-pro), trans-4,5-(methano)-L-proline (tMe-Pro), trans-4,5-(methano)-D-proline (tMe-pro);

AA₆=glycine (gly or Gly), sarcosine (Sar), azaglycine (Azagly);

AA₅-AA₆ together can be substituted by a β-turn mimetic, where what is meant by β-turn mimetic is a chemical structure which, by mimicking the central portion of the Pro-Gly β-turn, allows the molecule to take on a conformation useful for the formation of bonds with the protein MyD88, with formulas (VI) and (VII)

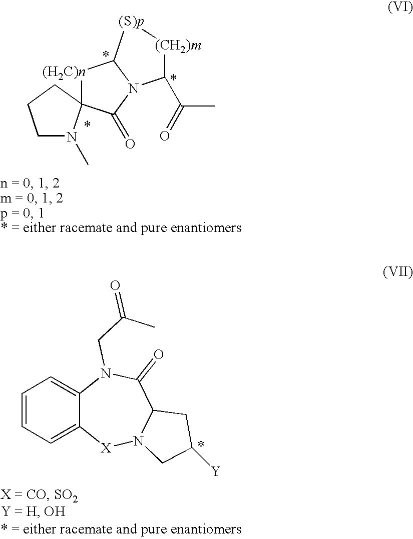

AA₇=is the residue of glycine (gly or Gly), azaglycine (Azagly), L-threonine (Thr), D-threonine (thr), L-cysteine (Cys), D-cysteine (cys), or is absent;

when AA₄=AA₇=Cys or cys there is a disulphide bridge between the two cysteines;

when some or all of AA₁, AA₂, AA₃, AA₄, AA₅, AA₆ and AA₇ are amino acids, these can be L or D and the sequence can be reversed or not;

the bond between the AA₁-AA₇ residues is always of the amide type;

the terminal amine group can be free or acylated with a pharmacologically acceptable radical useful for transporting the molecule, e.g., acetyl, formyl, benzoyl, propionyl, cyclohexyl, myristoyl; the terminal carboxyl can be in the form of carboxylic acid or primary amide.

the individual enantiomers, diastereoisomers, mixtures thereof and their pharmaceutically acceptable salts;

upon the following conditions:

that at least one of AA₁-AA₇ is not a natural amino acid among those indicated above, or

if all of AA₁-AA₇ are natural amino acids among those indicated above, said AA₁-AA₇ sequence is reversed.

The formula (I) compounds are useful as medicaments, particularly for the preparation of a medicament for the treatment of diseases deriving from dysregulation of the signalling of the TLR/IL-R1 receptor system, and particularly inflammatory and autoimmune diseases; cardiovascular and atherogenic diseases; sepsis and shock; and transplant rejection.

Therefore, the subjects of the present invention are formula (I) compounds, pharmaceutical compositions containing them and their use as medicaments.

### DETAILED DESCRIPTION OF THE INVENTION

The terminal amine group can be free or acylated with a pharmacologically acceptable radical useful for transporting the molecule, e.g., acetyl, formyl, benzoyl, propionyl, cyclohexyl, myristoyl; the terminal carboxyl can be in the form of carboxylic acid or primary amide. Examples of anions of pharmacologically acceptable acids are Cl⁻, Br⁻, I⁻ CH₃COO⁻, and CF₃COO⁻. Other pharmacologically acceptable anions can be selected by the expert in the field according to the normal criteria in use in the field, such as, for example, non-toxicity or virtual non-toxicity or in any event acceptable toxicity, or formulation advantages, such as, for example, solubility or crystalline form. What is meant by pharmacologically acceptable salt is any salt that does not give rise to toxic or unwanted effects or in which such effects manifest themselves in an acceptable form from the clinical point of view. In addition to his or her general knowledge, the technician of average experience can easily consult the literature, e.g. the European Pharmacopoeia or the United States Pharmacopeia.

Assuming the general definition of an arginino-mimetic group as indicated above, preferred examples are the following 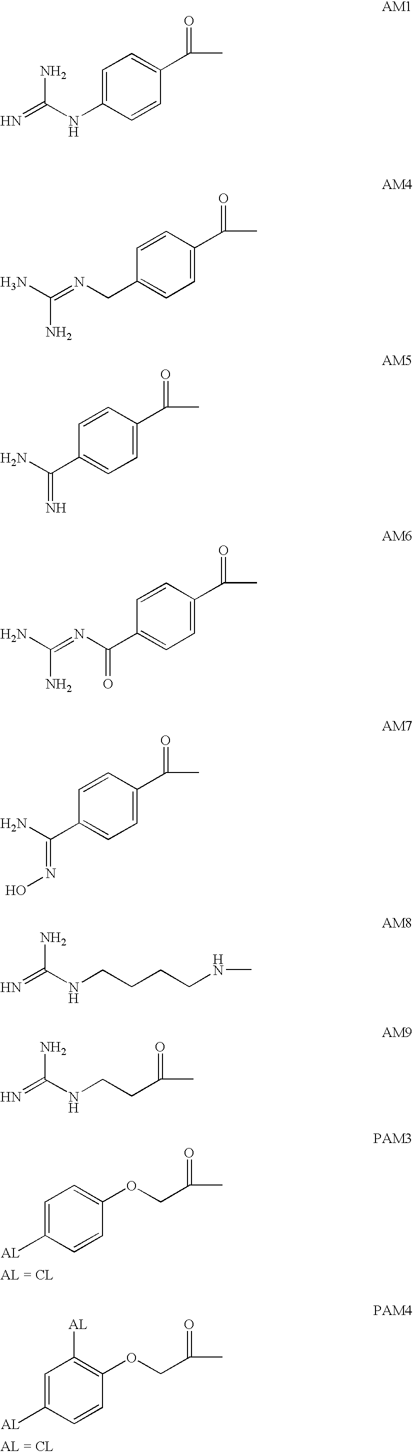 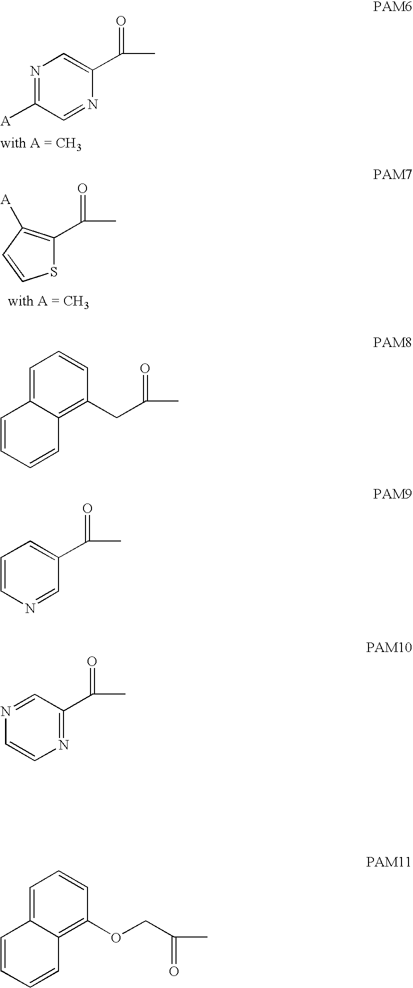
in which A is a straight or branched C₁-C₄ alkyl group; AL is a halogen group selected from F, Cl, Br and I.

Assuming the general definition of a spacer group as indicated above, preferred examples of this group have the formula (SPX)ₙ where n=0-3 and is as defined in the following table 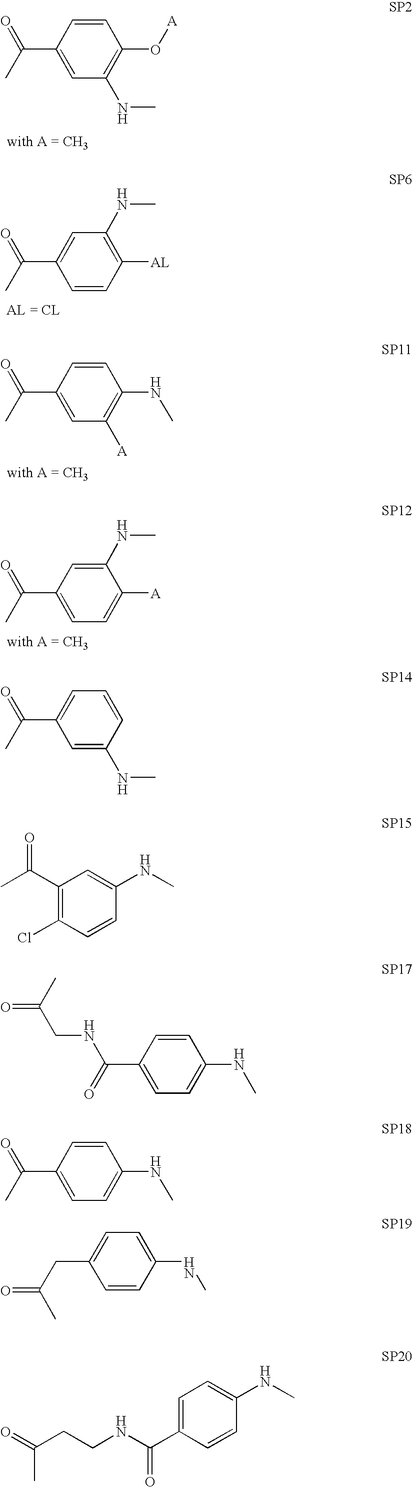 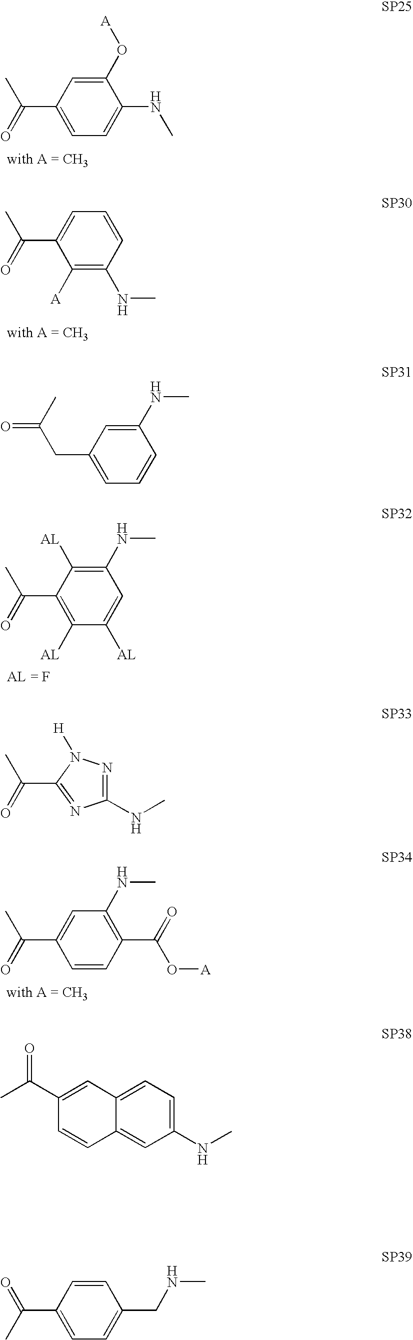
in which A is a straight or branched C₁-C₄ alkyl group; AL is a halogen atom selected from F. Cl, Br and I.

Assuming the general definition of a β-turn mimetic as indicated above, preferred examples of this group are the following

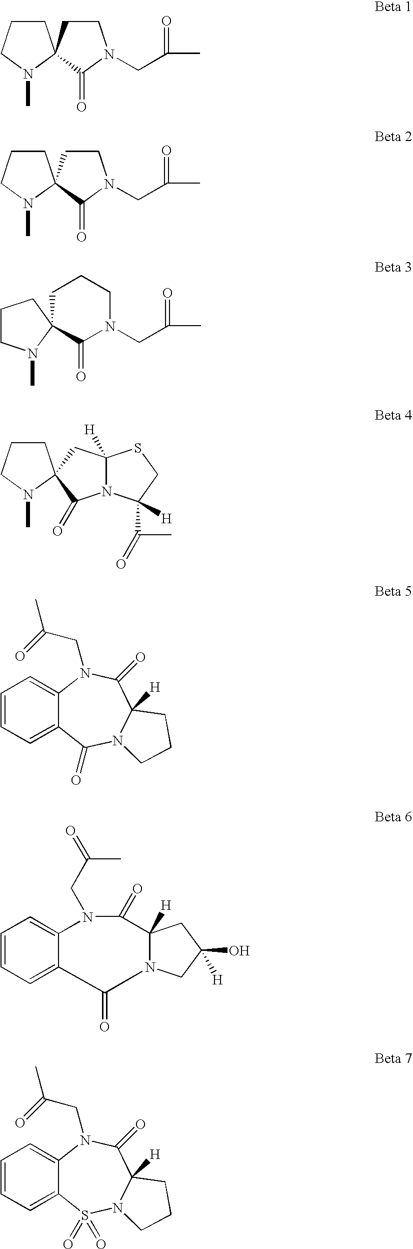

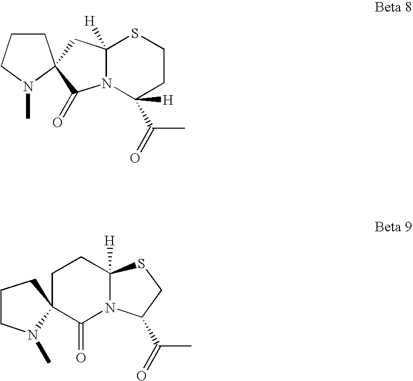

The compounds were subjected to three biological primary screening assays described in the experimental part: a) double hybrid assay, b) NF-κB inhibition assay and c) RGA assay. Compounds proving active in either of the three biological assays were regarded as active compounds.

The compounds represented by formula (I) can be divided into the following classes:

1. Peptidic compounds prepared according to Scheme 9 up to formula I^{V} or formula I^{VI}.

The preferred compounds are:

ST2565: Ac-thr-gly-pro-leu-val-asp-arg-NH₂

2. Peptidomimetic compounds, prepared according to Scheme 9 up to formula I^{II} or formula I^{III}.

The preferred compounds are: 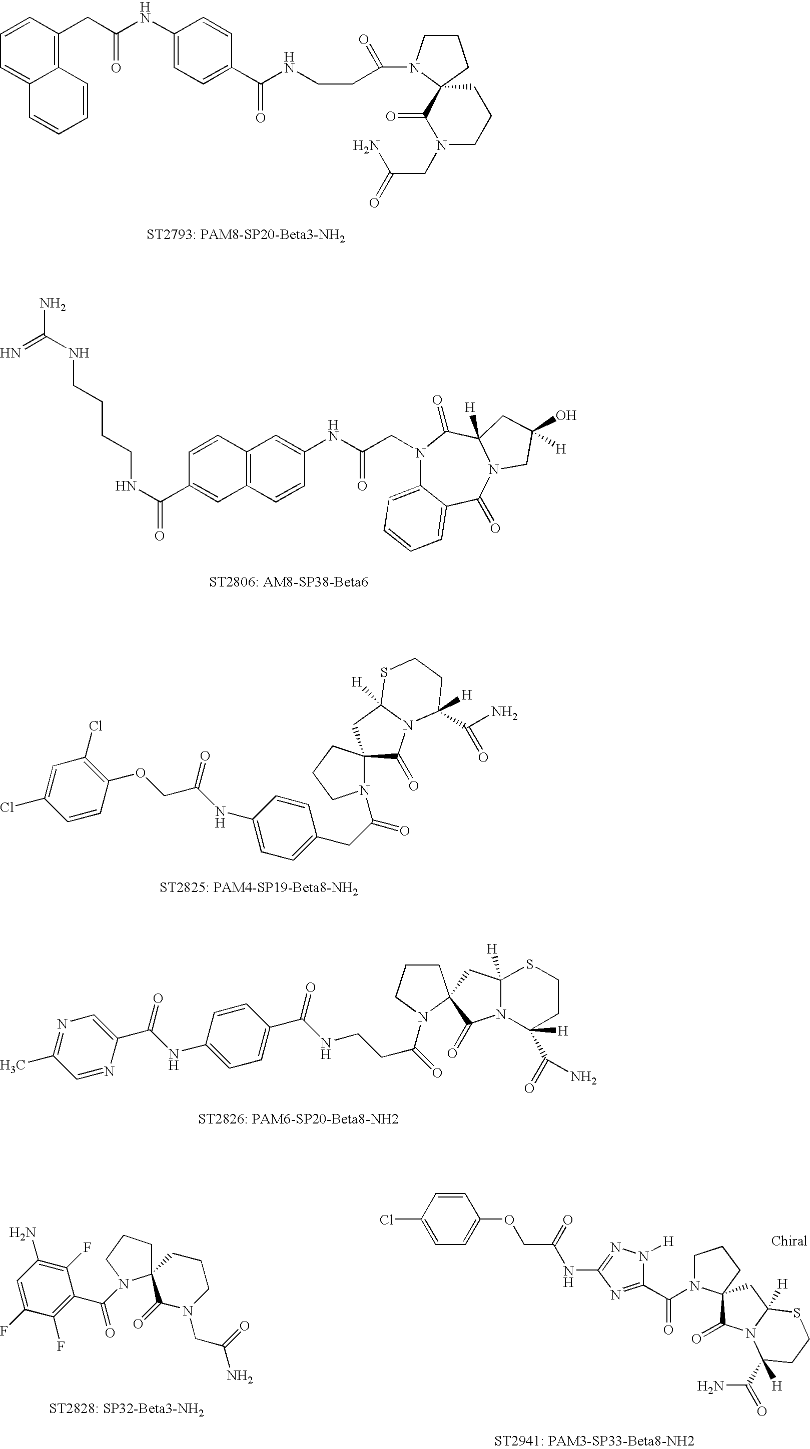 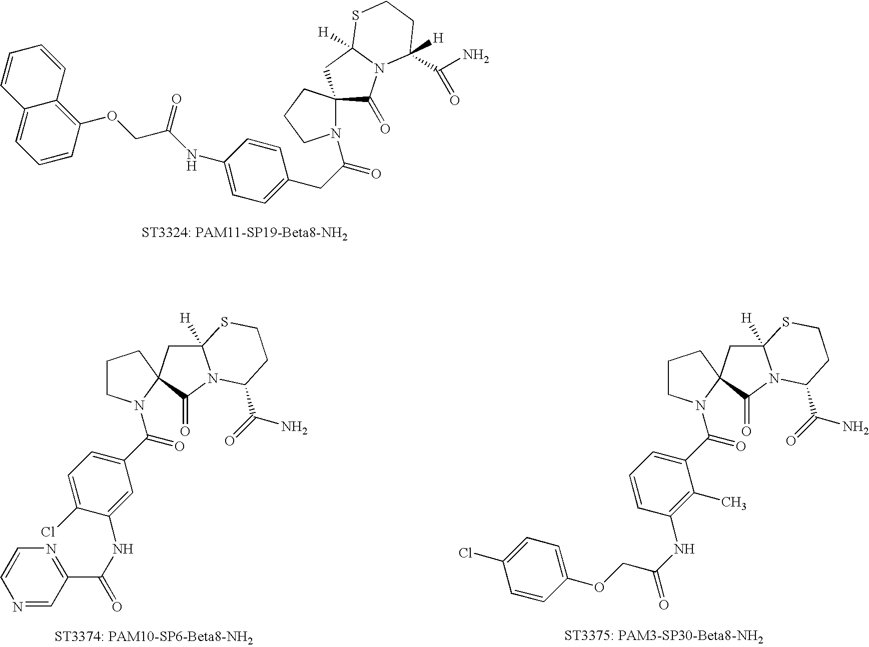
3. Partly peptidic compounds prepared according to Scheme 9 up to formulae I^{II}, I^{III}, I^{IV} and I^{V}, divided into the following subclasses:
Partly peptidic compounds in which AA₅-AA₆ are substituted with a β-turn mimetic, prepared according to Scheme 9 up to formulae I^{IV} and I^{V}.

The preferred compound is: 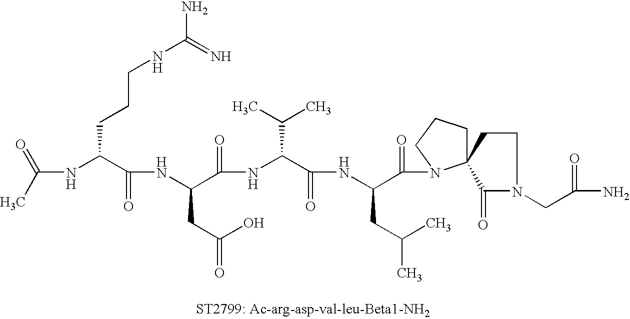
Partly peptidic compounds in which AA₂-AA₃-AA₄ are substituted with a spacer and AA₅-AA₆ are substituted with a β-turn, mimetic prepared according to Scheme 9 up to formulae I^{II} and I^{III}.

The preferred compounds are: 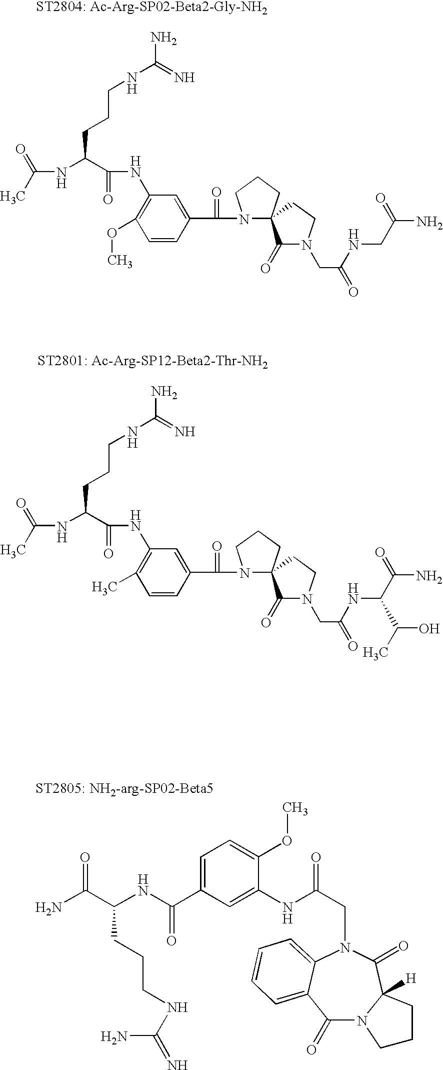
Partly peptidic compounds in which AA1 is an arginino-mimetic and AA₅-AA₆ is substituted with a β-turn mimetic, prepared according to Scheme 9 up to formulae I^{IV} and I^{V}.

The preferred compound is: 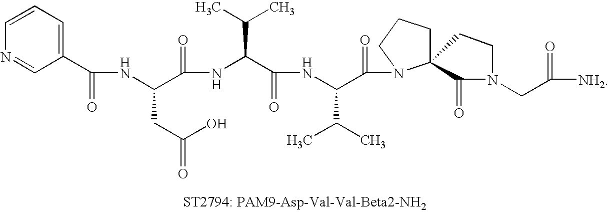
Partly peptidic compounds in which AA₁ is an arginino-mimetic and AA₂-AA₃-AA₄ are substituted with a spacer, prepared according to Scheme 9 up to formulae I^{III} and I^{IV}

The preferred compounds are:

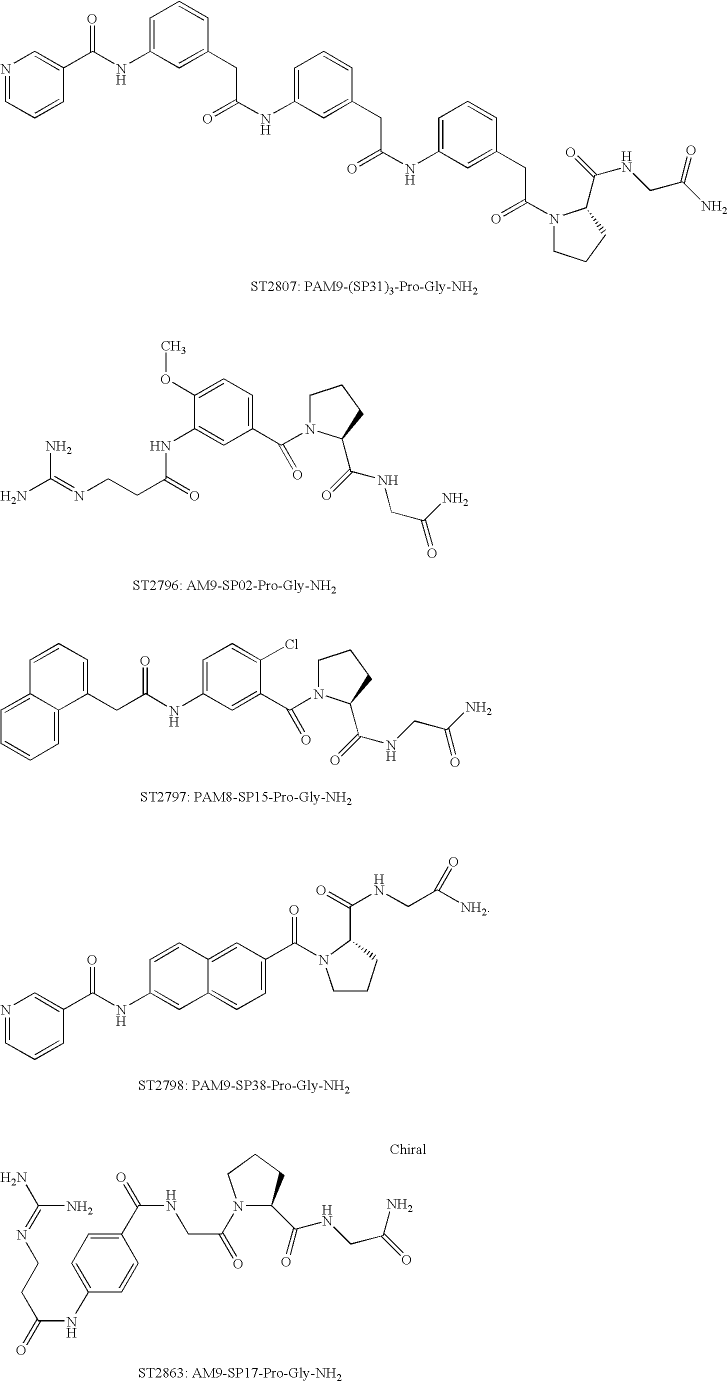

Partly peptidic compounds in which AA₁ is an arginino-mimetic, AA₂-AA₃-AA₄ are substituted with a spacer, AA₅-AA₆ are substituted with a β-turn mimetic and AA7 is an amino acid, prepared according to Scheme 9 up to formula I^{III}.

The preferred compound is:

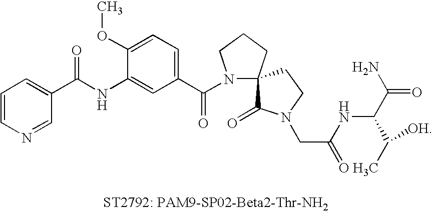

Partly peptidic compounds in which AA₂-AA₃-AA₄ are substituted with a spacer, prepared according to Scheme 9 up to formula I^{III}.

The preferred compound is:

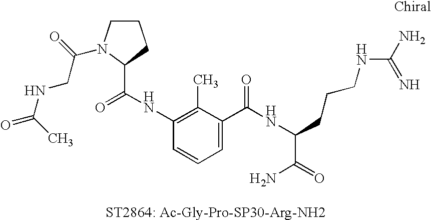

Partly peptidic compounds in which one or more amino acids are substituted with one or more aza-aminoacids, prepared according to Scheme 9 up to formula I^{V} and I^{VI}.

The preferred compounds are:

| | |
|---|---|
| ST2926 | H-Arg-Gly-AzaVal-Val-Pro-Gly-NH₂ |
| ST3032 | Ac-Azagly-Azagly-pro-leu-val-asp-arg-NH₂ |
| ST2927 | Ac-Arg-Asp-Azagly-Val-Pro-Gly-NH₂ |
| ST2930 | Ac-thr-Azagly-pro-leu-val-asp-arg-NH₂ |
| ST2920 | Ac-Arg-Asp-Val-AzaVal-Pro-Gly-NH₂ |
| ST2928 | Ac-Arg-Asp-AzaLeu-Val-Pro-Gly-NH₂ |

The compounds according to the present invention can be prepared by means of conventional synthesis methods with which experts in the field are familiar. The general peptide synthesis techniques are well suited to the purposes of the present invention. Indicative references are, for example: Norbert Sewald, Hans-Dieter Jakubke, Peptides: Chemistry and Biology, Wiley VCH (2002); Miklos Bodanszky, Principles of Peptide Synthesis (Sec. Ed.), Springer-Verlag (1993); John Jones, Amino Acid and Peptide Synthesis (Oxford Chemistry Primers), Oxford Science Publications (2000).

A general synthesis scheme is described in Scheme 9 presented here below and to which the reader is referred for a general description. When the compounds according to the present invention contain non-peptidic portions, their synthesis entails the construction of building blocks on which the peptidic molecule is then completed.

In the schemes described here below, the building blocks are exemplified for a number of preferred embodiments, it being understood that they serve as guides to the expert in the field for the embodiment of the present invention in its complete context. In fact, as regards those that are not explicitly exemplified, the expert in the field can supplement them by resorting to his or her general knowledge, for example, by finding starting compounds among those available on the market, or by preparing them by analogy with the ones outlined in the examples.

Synthesis of the Building Blocks

The portions of the peptidomimetic compounds called β-turn mimetics were synthesised using building blocks synthesised according to the synthesis methods described in the following examples:

The building block ST2364 (11), which is useful for the synthesis of peptidomimetic compounds containing the β-turn mimetic Beta2, was synthesised according to Scheme 1, using the method described by R. L. Johnson and co-workers (Genin, M. J.; et al.; J. Org. Chem.; 1993, 58, 2334-2337) modified according to R. D Long and K. D. Moeller (Long, R. D.; et al.; J. Am. Chem. Soc.; 1997, 119, 12394-1239) up to intermediate 8, and then using the method described in Example 1 here below.

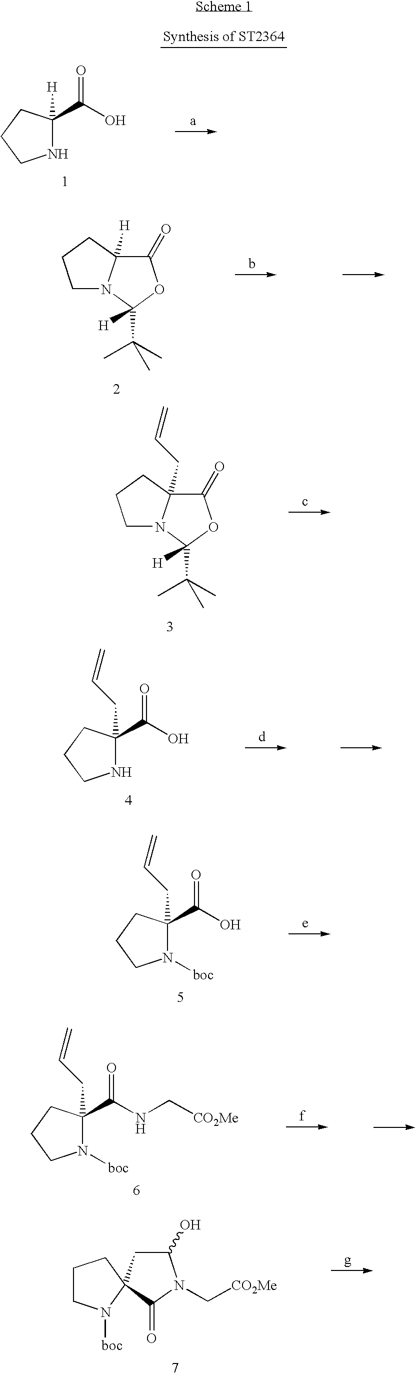

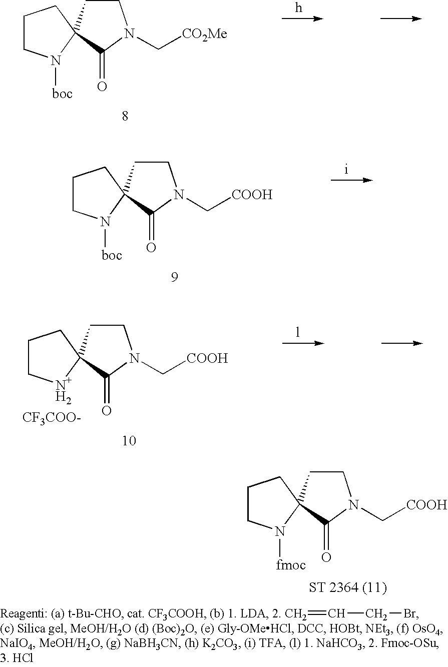

### EXAMPLE 1

### Preparation of {(5R)-1-[(9H-fluoren-9-yl-methoxy)-carbonyl]-6-oxo-1,7-diazaspiro[4.4]non-7-yl}-acetic acid ST2364 (11)

### Preparation of the Intermediate [(5R)-1-(terz-butoxycarbonyl)-6-oxo-1,7-diazaspiro[4.4]non-7-yl]-acetic acid (9)

8.5 g (0.027 mol) of (5R)-7-(2-methoxy-2-oxo-ethyl)-6-oxo-1,7-diazaspiro[4.4]nonan-1-carboxylic acid terz-butyl ester (8) are solubilised in 140 ml of H₂O and 140 ml di MeOH. To the solution are added 7.48 g (0.054 mol) of K₂CO₃ and the mixture is left to stir overnight at room temperature. It is then acidified to pH 5 with HCl 2 N and evaporated under reduced pressure. The residue obtained is dissolved in H₂O, the pH is lowered to 2-3 and extraction is done with CH₂Cl₂. The organic phase is anhydrified with Na₂SO₄ and brought to dryness under reduced pressure, obtaining 6.5 g of a glassy solid (yield: 81%).

TLC: CHCl₃ 8/MeOH 2/AcOH 0.1; RF: 0.54.

¹HNMR (300 MHz, CDCl3): δ 1.30-1.60 (2s, 9H), 1.80-2.20 (m, 4H), 2.20-2.40 (m, 2H), 2.40-2.60 (m, 2H), 3.30-3.70 (m, 4H), 3.85 (d, 1H), 3.20-4.20 (sa, 1H), 4.50 (d, 1H).

### Preparation of the Intermediate (5R)-7-(carboxymethyl)-6-oxo-7-aza-1-azoniaspiro[4.4]nonano-trifluoroacetate (10)

6g (0-02 mol) of [(5R)-1-(terz-butoxycarbonyl)-6-oxo-1,7-diazaspiro-[4.4]non-7-yl]-acetic acid (9) are solubilised in 100 ml of CH₂Cl₂ and 100 ml of trifluoroacetic acid. The solution is held under stirring at room temperature for 1 hour, after which it is brought to dryness under reduced pressure and H₂O is added to the residue and evaporated, again under reduced pressure, to remove any traces of trifluoroacetic acid. The oil obtained is dried with an oil pump, yielding 6.2 g of an oily product (yield: 100%).

TLC: CHCl₃ 60/MeOH 40/H₂O 15/iPrOH 10/AcOH 15; RF: 0.33.

¹HNMR (300 MHz, DMSOd₆): δ 2.10 (m, 4H), 2.30 (m, 2H), 3.35 (m, 2H), 3.50 (m, 2H), 4.05 (2d, 2H), 9.25 (sa, 1H), 9.35 (sa, 1H).

### Preparation of {(5R)-1-[(9H-fluoren-9-yl-methoxy)-carbonyl]-6-oxo-1,7-diazaspiro[4.4]non-7-yl}-acetic acid ST2364 (11)

6.2 g (0.02 mol) of (5R)-7-(carboxymethyl)-6-oxo-7-aza-1-azonia-spiro[4.4]nonano-trifluoroacetate (10) and 4 g (0.047 mol) of NaHCO₃ are solubilised in 200 ml of H₂O. 7.0 g (0.021 mol) of Fmoc-N—OSu dissolved in 300 ml of acetone are then added to the solution and the solution is left to stir at room temperature for 20 hours. H₂O is added to the reaction mixture which is then washed three times with Et₂O. The aqueous phase is brought to pH 2-3 with HCl 2N and extraction is performed with CHCl₃. The organic phase is anhydrified with anhydrous Na₂SO₄ and evaporation performed under reduced pressure. The pitchy solid obtained is crystallised with CHCl₃, yielding 6.1 g of a white solid (yield: 73%).

TLC: CHCl₃ 8/MeOH 2/AcOH 0.1; RF: 0.55.

MP: 144°-146° C.

[α]_{D}:−12.6; conc. 0.5% in MeOH.

HPLC: Column: μBondapack C18 3.9×150 mm;

Mobile phase: KH₂PO₄50 mM./CH₃CN 75/25;

Flow rate: 1.0 ml/min. r. temp.;

R.T.: 10.9 min.

¹HNMR (300 MHz, DMSOd₆): δ 1.50 (m, 1H), 1.60-2.00 (m, 4H), 2.40 (q, 1H), 2.60, 3.02 (2m, 1H), 3.30 (m, 2H), 3.40 (m, 2H), 3.70, 4.10 (2d, 2H), 4.15, 4.20 (2m, 1H), 4.35, 4.75 (2dd, 1H), 7.25-7.45 (m, 4H), 7.55-7.70 (m, 2H), 7.83 (d, 2H) 12.70 (sa, 1H).

The building block ST2201, useful for the synthesis of the peptidomimetic compounds containing the β-turn mimetic Beta1, was synthesised according to Scheme 1, by the same method used to synthesise ST 2364 (Example 1), starting from D-proline as the starting product rather than from L-proline. The analytical data of ST2201 are described in Example 2 here below.

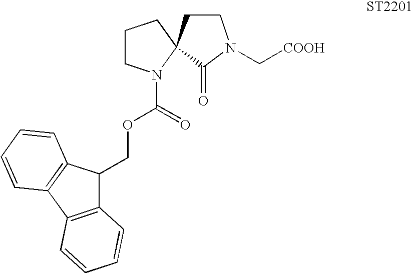

### EXAMPLE 2

### Preparation of {(5S)-1-[(9H-fluoren-9-yl-methoxy) -carbonyl]-6-oxo-1,7-diazaspiro[4.4]non-7-yl}-acetic acid ST2201

TLC: CHCl₃ 8/MeOH 2; RF: 0.33.

MP: 138°-141° C.

[α]_{D}:+15.1; conc. 0.5% in MeOH.

HPLC: Column: μBondapack C18 3.9×150 mm;

Mobile phase: KH₂PO₄ 50 mM/CH₃CN 65/35;

Flow rate: 1.0 ml/min. r. temp.;

R. T.: 8.2 min.

¹HNMR (300 MHz, DMSOd₆): δ 1.50 (m, 1H), 1.60-2.00 (m, 4H), 2.40 (q, 1H), 2.60, 3.02 (2m, 1H), 3.30 (m, 2H), 3.40 (m, 2H), 3.70, 4.10 (2d, 2H), 4.15, 4.20 (2m, 1H), 4.35, 4.75 (2dd, 1H), 7.25-7.45 (m, 4H), 7.55-7.70 (m, 2H), 7.83 (d, 2H) 12.70 (sa, 1H).

The building block ST2451 (22), useful for the synthesis of the peptidomimetic compounds containing the β-turn mimetic Beta3, was synthesised according to Scheme 2, using the method described by R. L. Johnson and co-workers (Genin, M. J.; et al.; J. Med. Chem.; 1999, 42, 628-637) up to intermediate 16, and then using the method described in Example 3 here below.

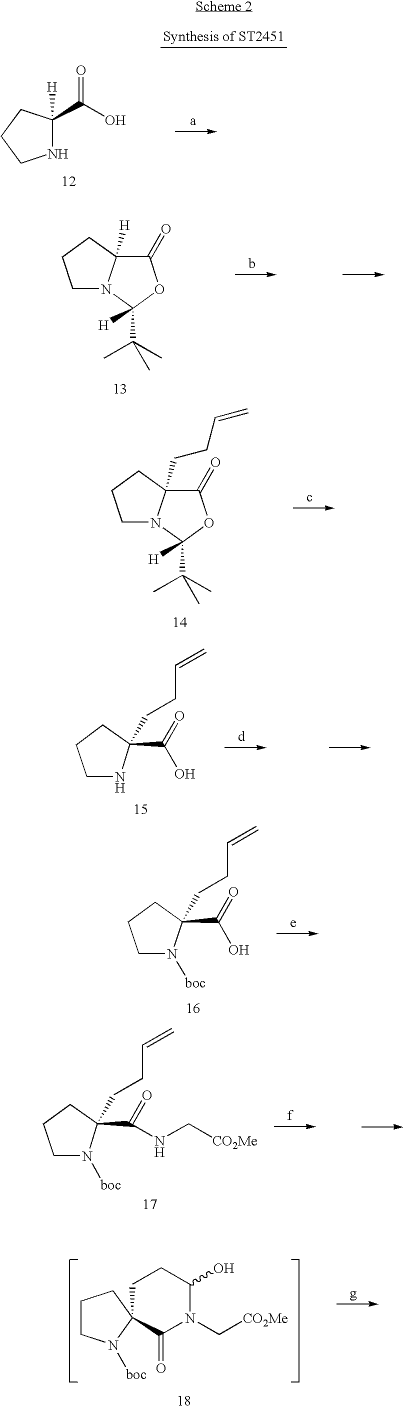

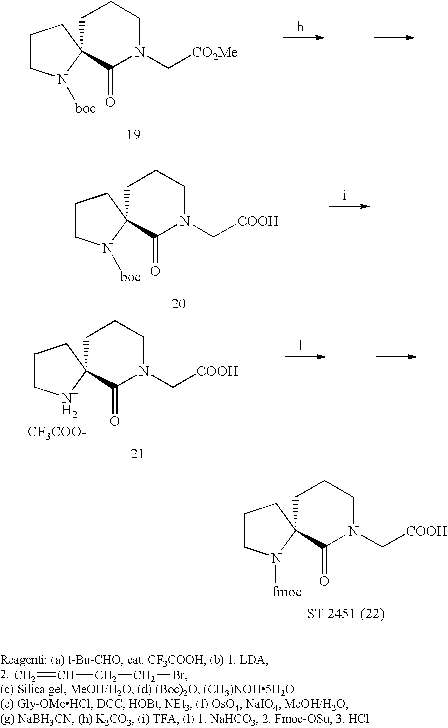

### EXAMPLE 3

### Preparation of {(5S)-1-[(9H-fluoren-9-yl-methyloxy)-carbonyl]-6-oxo-1,7-diazaspiro[4.5]dec-7-yl}-acetic acid ST2451 (22)

### Preparation of the Intermediate 2-but-3-en1-yl-1-(terz-butoxy-carbonyl)-L-prolylglycine methylester (17)

8.0 g (0.0297 mol) of 2-but-3-en-1-yl-1-(terz-butoxycarbonyl)-L-proline (16), 3.7 g (0.0297 mol) of glycine methylester hydrochloride and 4.0 g (0.0297 mol) of hydroxybenzotriazole are solubilised in 100 ml of anhydrous CHCl₃. To the solution are added 4.1 ml (0.0297 mol) of TEA and then 6.1 g (0.0297 mol) of DCC dissolved in 100 ml pf anhydrous CHCl₃. The reaction mixture is left to stir overnight at room temperature in an N₂ atmosphere. The dicyclohexylurea (DCU) formed is filtered and the filtrate is brought to dryness at reduced pressure. The residue thus obtained is shaken with Et₂O and filtered to eliminate any DCU and the liquid phase is washed with NaHCO₃ 1M, salt H₂O, and 10% citric acid and then again with salt H₂O. The organic phase is anhydrified with anhydrous Na₂SO₄ and brought to dryness at reduced pressure, obtaining 13 g of a yellow oil that is purified using a silica gel chromatography column, eluting with n-hexane/AcOEt 2:1. The purification yields 9.4 g of a white solid (yield: 93%).

TLC: hexane 2/AcOEt 1; RF: 0.27.

¹H-NMR (200 MHz, CDCl₃): δ 1.54, 1.60 (2s, 9H), 1.75 (m, 3H), 2.10 (m, 4H), 2.75 (m, 1H), 3.35 (m, 1H), 3.58 (m, 1H), 3.75 (s, 3H), 4.05 (m, 2H), 5.00 (m, 2H), 5.82 (m, 1H), 6.50, 8.32 (2sa, 1H).

### Preparation of the Intermediate (5S)-7-(2-methossy-2-oxoethyl)-6-oxo-1,7-diazaspiro[4.5]decano-1-carboxylic acid terz-butyl ester (19)

9.15 g (0.027 mol) of 2-but-3-en-1-yl-1-(terz-butoxycarbonyl)-L-prolylglycine methylester (17) are solubilised in 300 ml di MeOH/H₂O 2:1. 0.33 g (0.0013 mol) of OSO₄ are added to the solution, which is then left to stir for 10 minutes after bubbling N₂, whereupon 17.1 g (0.08 mol) of NaIO₄ are added portion-wise. A white precipitate is formed from the dark-coloured solution and left to stir at room temperature for 24 hours. H₂O is added to the reaction mixture until a solution is obtained which is extracted several times with AcOEt. The pooled organic phases are washed with H₂O, anhydrified with anhydrous Na₂SO₄ and brought to dryness under reduced pressure, obtaining 9.2 g of a mixture of the diastereoisomers of (5R)-8-hydroxy-7-(2-methoxy-2-oxoethyl)-6-oxo-1,7-diaza-spiro[4.5]-decano-1-carboxylic acid terz-butyl ester (18) as a dark-coloured oil which is reacted without any further purification.

TLC: AcOEt; RF: 0.39 and 0.52 (diastereoisomers).

9.0 g (0.026 mol) of (5R)-8-hydroxy-7-(2-methoxy-2-oxoethyl)-6-oxo-1,7-diazaspiro[4.5]decano-1-carboxylic acid terz-butyl ester (18) are solubilised in 180 ml of anhydrous THF. 18 ml of trifluoroacetic acid are added to the solution and the solution is cooled with an ice bath and 4.68 g (0.074 mol) of NaBH₃CN are added. The reaction mixture is left to stir for 20 hours at room temperature under a nitrogen atmosphere and then alkalinised with K₂CO₃. The solution is separated from the residue by filtration and the filtrate is brought to dryness under reduced pressure. The amorphous mass obtained is dissolved in H₂O and extracted several times with CH₂Cl₂. The organic phase is anhydrified with anhydrous Na₂SO₄ and brought to dryness under reduced pressure. 9 g of a dark-coloured oil are obtained which is purified by silica column chromatography, eluting with AcOEt/n-hexane 3:1. 4.5 g of a light-coloured oil are obtained (yield: 53%).

TLC: AcOEt; RF: 0.5.

¹H-NMR (300 MHz, CDCl₃): δ 1.40, 1.60 (2s, 9H), 1.60-2.15 (m, 6H), 2.15-3.80 (m, 2H), 3.30 (m, 1H), 3.30-3.80 (m, 4H), 3.75, 3.76 (2s, 3H), 4.70, 4.78 (2d, 1H).

### Preparation of the Intermediate [(5S)-1-(terz-butoxycarbonyl) -6-oxo-1,7-diazaspiro[4.5]dec-7-yl]-acetic acid (20)

4 g (0.012 mol) of (5S)-7-(2-methoxy-2-oxoethyl)-6-oxo-1,7-diazaspiro[4.5]decano-1-carboxylic acid terz-butyl ester (19) are dissolved in 60 ml of MeOH and 60 ml of H₂O; 3.32 g (0.024 mol) of K₂CO₃ are then added. The reaction mixture is held under stirring at room temperature for 20 hours, then brought to pH 5 with HCl 2N and brought to dryness under reduced pressure. The residue obtained is solubilised in H₂O, brought to pH 2-3 and extracted several times with CH₂Cl₂. The organic phase is anhydrified on anhydrous Na₂SO₄ and brought to dryness under reduced pressure, obtaining 3.2 g of a white solid (yield: 86%).

TLC: CHCl₃ 8/MeOH 2/CH₃COOH 0.1; RF: 0.59.

¹H-NMR (300 MHz, CDCl₃): δ 1.43 (s, 9H), 1.60-2.15 (m, 6H), 2.15-2.50 (m, 2H), 3.25 (m, 1H), 3.30-3.80 (m, 4H), 4.43, 4.98 (2d, 1H).

### Preparation of the Intermediate (5R)-7-(carboxymethyl)-6-oxo-7-aza-1-azoniaspiro[4.5]decano-trifluoroacetate (21)

3.1 g (0.01 mol) of [(5S)-1-(terz-butoxycarbonyl)-6-oxo-1,7-diazaspiro-[4.5]dec-7-yl]-acetic acid (20) are solubilised in 60 ml of CH₂Cl₂ and 60 ml of trifluoroacetic acid; the reaction solution is left to stir at room temperature for 1 hour. It is then brought to dryness under reduced pressure at 30° C., the residue is taken up with H₂O and again brought to dryness under reduced pressure, drying thoroughly with an oil pump. 3.1 g of a light-coloured oil are thus obtained (yield: 95%).

TLC: CHCl₃ 60/MeOH 40/H₂O 15/isoPrOH/10/AcOH 15; RF: 0.4.

¹H-NMR (200 MHz, D₂O): δ 1.85-2.30 (m, 8H), 3.20-3.60 (m, 4H), 4.05 (d, 2H).

### Preparation of {(5S)-1-[(9H-fluoren-9-yl-metoxy)-carbonyl]-6-oxo-1,7-diazaspiro[4.5]dec-7-yl}-acetic acid ST2451 (22)

3.2 g (0.01 mol) of (5R)-7-(carboxymethyl)-6-oxo-7-aza-1-azoniaspiro-[4.5]decano-trifluoroacetate (21) are dissolved in 100 ml of H₂O; 1.7 g (0.02 mol) of NaHCO₃ are added to the solution and then 3.7 g (0.011 mol) of Fmoc-N—OSu dissolved in 150 ml of acetone. The reaction mixture is left to stir at room temperature for 24 hours, the acetone is evaporated under reduced pressure, followed by dilution with H₂O and washing with Et₂O. The aqueous phase is brought to pH 2-3 with HCl 2N and extracted with CHCl₃; the organic phase is anhydrified on anhydrous Na₂SO₄ and brought to dryness under reduced pressure. The product obtained is crystallised with CH₂Cl₂ and Et₂O, thus obtaining 1.4 g of a white solid (yield: 32%).

TLC: CHCl₃ 8/MeOH 2/CH₃COOH 0.1; RF: 0.62.

MP: 122° C.

[α]_{D}:−26.4 (0.5% in MeOH).

E.A.: Theoretical: C, 69.10; H, 6.03; N, 6.44;

Found: C, 67.81; H, 5.90; N, 6.31.

HPLC: Column: Symmetry C18 (5μ) 3.9×150 mm;

Mobile phase: KH₂PO₄ 50 mM pH 3/CH₃CN 65/35;

Flow rate: 1.0 ml/min, r. temp.;

R.T.: 5.2 min.

¹H-NMR (300 MHz, CDCl₃): δ 1.20, 1.38, 1.62 (3m, 1H), 1.78-2.20 (m, 5H), 2.22-2.50 (m, 2H), 2.80, 3.45 (2m, 1H), 3.25 (m, 1H), 3.60 (dd, 1H), 3.65 (m, 1H), 3.80, 3.85 (2d, 1H), 4.15, 5.2 (2m, 1H), 4.2-4.35 (m, 1H) 4.40 (m, 1H), 4.60 (m, 1H), 7.20-7.45 (m, 8H), 7.58 (m, 2H), 7.75 (d, 2H).

The building block ST2535 (34), useful for the synthesis of the peptidomimetic compounds containing the β-turn mimetic Beta4, was synthesised according to Scheme 3, using the method described by R. L. Johnson and co-workers (Genin, M. J.; et al.; J. Med. Chem.; 1999, 42, 628-637) up to intermediate 31, and then using the method described in Example 4 here below.

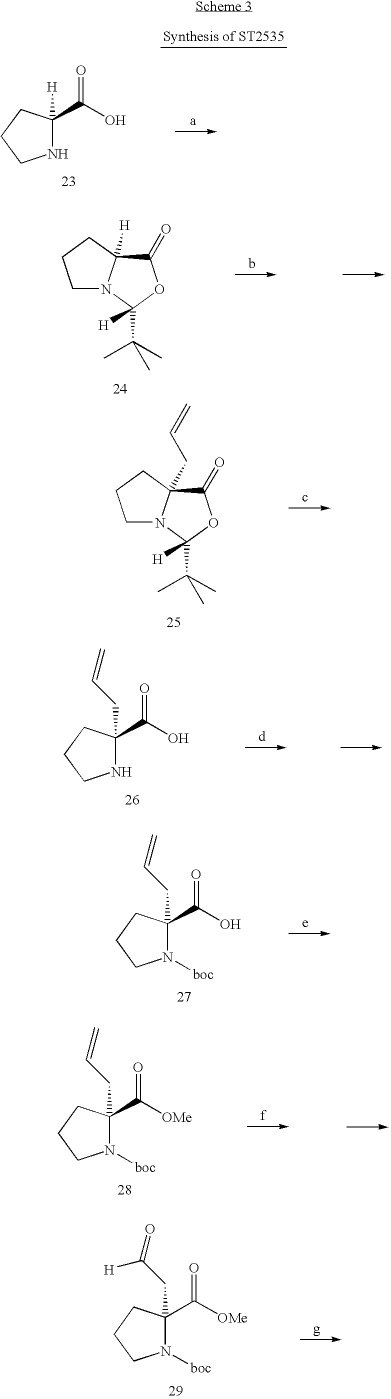

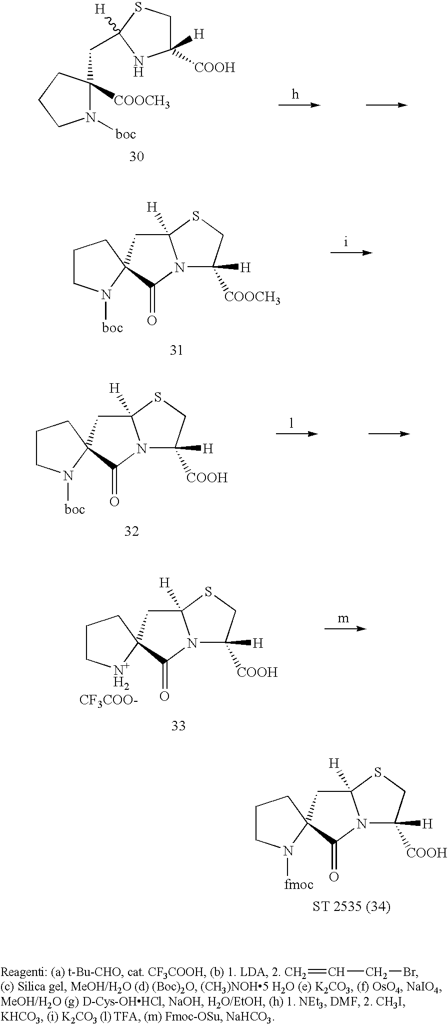

### EXAMPLE 4

### Preparation of (2R,3′S,7a′R)-1-[(9H-fluoren-9-yl-methoxy)-carbonyl]-5′-oxotetrahydrospiro-[pyrrolidine-2,6′-pyrrole-[2,1-b][1,3]thiazole]-3′-carboxylic acid ST2535 (34)

### Preparation of the Intermediate (2R,3′S,7a′R)-1-(terz-butoxy-carbonyl)-5′-oxotetrahydrospiro-[pyrrolidine-2,6′-pyrrole[2,1-b][1,3]thiazole]-3′-carboxylic acid (32)

5 g (0.014 mol) of (2R,3′S,7a′R)-5′-oxotetrahydro-1H-spiro-[pyrrolidine-2,6′-pyrrole[2,1-b][1,3]thiazole]-1,3′-dicarboxylic acid 1-terz-butyl 3′-methylester (31) are dissolved in 65 ml of H₂O and 65 ml of MeOH. 3.87 g (0.028 mol) of K₂CO₃ are added to the solution. The suspension is left to stir at room temperature for 20 hours, the pH of the mixture is lowered to 5 with HCl 2N and the mixture is brought to dryness under reduced pressure. The residue is taken up with H₂O, brought to pH 2-3 and extracted with CHCl₃. The organic phase is washed with H₂O, dried on Na₂SO₄ and brought to dryness under reduced pressure. The glassy residue obtained is crystallised with Et₂O to give 3 g of a white solid (yield: 64%).

TLC: CHCl₃ 8/MeOH 2/CH₃COOH 0.1; RF: 0.56.

¹H-NMR (200 MHz, CDCl₃): δ 1.45 (s, 9H) 1.75-2.45 (m, 5H), 2.85 (dd, 1H) 3.50 (m, 4H) 4.98 (m, 1H), 5.20, 5.30 (d and dd, 1H), 5.00-6.00 (sa, 1H).

### Preparation of the Intermediate (2R,3′S,7a′R)-3′-carboxy-5′-oxotetrahydrospiro[pyrrolidine-2,6′-pyrrole[2,1-b][1,3]thiazole]trifluoroacetate (33)

3.0 g (0.0087 mol) of (2R,3′S,7a′R)-1-(terz-butoxycarbonyl)-5′-oxotetrahydrospiro[pyrrolidine-2,6′-pyrrole[2,1-b]-[1,3]thiazole]-3′-carboxylic acid (32) are solubilised in 60 ml of CH₂Cl₂ and 60 ml of trifluoroacetic acid. The whole is left to stir at room temperature for 2 hours and evaporated at 30° C.; the oil obtained is dried with an oil pump until a pitch is obtained that is crystallised with CHCl₃, giving 2.4 g of a white solid (yield: 80%).

TLC: CHCl₃ 60/MeOH 40/H₂O 15/isoPrOH 10/AcOH 15; RF: 0.57.

¹H-NMR (200 MHz, D₂O): δ 1.95-2.40 (m, 4H), 2.45 (dd, 1H), 2.90 (dd, 1H), 3.25-3.35 (m, 3H) 3.45 (dd, 1H), 4.93 (dd, 1H), 5.22 (t, 1H).

### Preparation of (2R,3′S,7a′R)-1-[(9H-fluorenyl-9-yl-methoxy)-carbonyl]-5′-oxotetrahydrospiro[pyrrolidine-2,6′-pyrrole[2,1-b][1,3]thiazole-]-3′-carboxylic acid ST2535 (34)

2.3 g (0.0064 mol) of (2R,3′S,7a′R)-3′-carboxy-5′-oxotetrahydro-spiro-[pyrrolidine-2,6′-pyrrole[2,1-b][1,3]thiazole]trifluoroacetate (33) are solubilised in 100 ml of H₂O; 1.1 g (0.013 mol) of NaHCO₃ are added to the solution and then 4.4 g (0.013 mol) of Fmoc-N—OSu dissolved in 150 ml of acetone. The whole is left to stir at room temperature for 20 hours and the acetone is evaporated under reduced pressure. More water is added and the aqueous solution is washed with Et₂O, then brought to pH 2-3 and extracted with CHCl₃. The organic phase is separated and anhydrified on anhydrous Na₂SO₄ and brought to dryness under reduced pressure. The glassy solid thus obtained is crystallised with ethyl acetate, obtaining 2.3 g of a filterable white solid (yield: 80%).

TLC: CHCl₃ 8/MeOH 2/CH₃COOH 0.1; RF: 0.67.

MP: 110° C. decomp.

E.A.: Theoretical:+8.4% H₂O═C, 59.21; H, 5.71; N, 5.52 S, 6.32;

Found:=C, 57.11; H, 4.87; N, 5.21: S, 5.43.

[α]²⁰_{D}:+121.9.

¹H-NMR (300 MHz, CDCl₃): δ1.60-2.20 (m, 4H), 2.30 (m, 1H), 2.80 (m, 1H), 3.30, 3.35-3.60 (3m, 4H), 4.05, 4.20 (2m, 1H), 4.40 (m, 2H), 4.60, 4.98(2m, 1H), 5.05, 5.15 (2m, 1H), 6.00-7.20 (sa, 1H), 7.20-7.45 (m, 8H), 7.58 (m, 2H), 7.75 (d, 2H).

HPLC: Column: Symmetry C18 (5) 3.9×150 mm;

Mobile phase: KH₂PO₄ 50 mM/CH₃CN 65/35;

Flow rate: 1.0 ml/min. r. temp.;

R.T.: 12.1 min.

The building block ST2304 (40), useful for the synthesis of the peptidomimetic compounds containing the β-turn mimetic Beta6, was synthesised according to Scheme 4, using the method described by M. R. Peña and J. K. Stille (J. Am. Chem. Soc.; 1989, 111, 5417-5424) up to intermediate 37 and then using the method described in Example 5 here below.

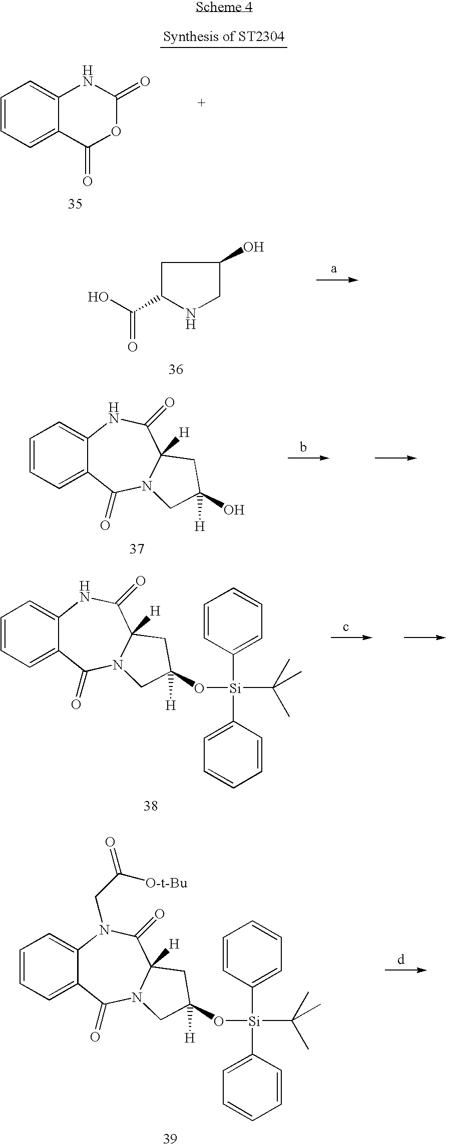

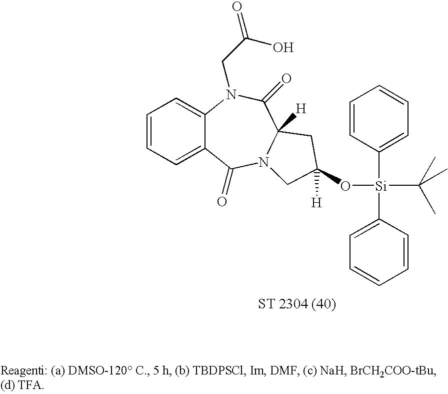

### EXAMPLE 5

### Preparation of ((2R,11aS)-2-{[terz-butyl(diphenyl)silyl]-oxy}-5,11-dioxo-2,3,11,11a-tetrahydro-1H-pyrrole[2,1-c][1,4]benzodiazepin-10(5H)-yl)-acetic acid ST 2304 (40)

### Preparation of the Intermediate (2R,11aS)-2-{[terz-butyl(diphenyl)-silyl]oxy}-2,3-dihydro-1H-pyrrole[2,1-c][1,4]benzodiazepine-5,11(10-H,11aH)-dione (38)

2.3 g (0.01 mol) of (2R,11aS)-2-hydroxy-2,3-dihydro-11H-pyrrole[2,1-c][1,4]benzodiazepine-5,11(10H,11aH)-dione (37) are solubilised in 30 ml of DMF; 3.4 g (0.05 mol) of imidazole are added and 5.6 ml (0.022 mol) of terz-butyl-diphenyl-silylchloride are added to this solution dropwise under stirring. The solution is left to stir at room temperature for 3.5 ore, and then 100 ml of H₂O are added and extraction is done with 100 ml of CH₂Cl₂ washing the organic phase with H₂O. The organic phase is anhydrified with anhydrous Na₂SO₄ and brought to dryness under reduced pressure, also using a mechanical pump, obtaining a dense oil which is solubilised hot in n-hexane/AcOEt and reprecipitated several times. After drying with the mechanical pump 3.9 g of an amorphous solid are obtained (yield: 83%).

TLC: n-hexane 1/AcOEt1; RF: 0.5.

E.A.: Theoretical: C, 71.45; H, 6.42; N, 5.95;

Found: C, 70.47; H, 6.67; N, 5.53.

¹H-NMR: (200 MHz, CDCl₃): δ 1.03 (s, 9H), 2.20 (m, 1H), 2.78 (m, 1H), 3.55, 3.60 (2d, 1H), 3.85, 3.90 (2d, 1H), 4.25 (m, 1H), 4.55 (m, 1H), 7.00 (d, 1H), 7.25-7.55 (m, 8H), 7.60-7.80 (m, 4H), 8.02 (d, 1H), 8.65 (s, 1H).

### Preparation of the Intermediate of [(2R,11aS)-2-{[terz-butyl-(diphenyl)silyl]oxy}-5,11-dioxo-2,3,11,11a-tetrahydro-1H-pyrrole-[2,1-c]-[1,4]benzodiazepin-10(5H)-yl)-acetic acid terz-butyl ester (39)

330 mg (0.008 mol) of 60% NaH are suspended in 6 ml of anhydrous THF after washing several times with THF. The suspension is cooled to −40° C. and 3.45 g (0.0073 mol) of (2R,11aS)-2-{[terz-butyl(diphenyl)-silyl]oxy}-2,3-dihydro-1H-pyrrol[2,1-c][1,4]benzodiazepine-5,11(10H, 11aH)-dione (38) solubilised in 18 ml of THF are added dropwise, maintaining the solution under stirring. After 45 minutes 1.2 ml (0.008 mol) of terz-butyl-bromoacetate are also added dropwise and the mixture is left to react under stirring for 2 hours, allowing the temperature to rise to room temperature. The suspension is then poured into 50 ml of H₂O and extracted twice with 30 ml of CH₂Cl₂ washing the pooled organic phases several times with H₂O and salt and anhydrifying with anhydrous Na₂SO₄. The solution is brought to dryness under reduced pressure obtaining an oil which, after washings with petroleum ether and drying with a mechanical pump, gives 3.6 g of an amorphous solid (yield: 84%).

TLC: n-hexane 7/AcOEt:3; R.F. 0.5.

E.A.: Theoretical: C, 69.83; H, 6.89; N, 4.79;

Found: C, 68.87; H, 7.37; N, 4.01.

¹H-NMR: (200 MHz, CDCl₃): δ 1.03 (s, 9H), 1.45 (s, 9H), 2.15 (m, 1H), 2.80 (m, 1H), 3.55, 3.60 (2d, 1H), 3.75, 3.82 (2d, 1H), 4.30 (m, 1H), 4.05-4.60 (dd, 2H), 4.62 (m, 1H), 7.20 (d, 1H), 7.30-7.60 (m, 8H), 7.60-7.80 (m, 4H), 8.00 (d, 1H).

### Preparation of ((2R11aS)-2-{[terz-butyl(diphenyl)silyl]oxy}-5,11-dioxo-2,3,11,11a-tetrahydro-1H-pyrrole[2,1-c][1,4]benzodiazepin-10(5H)-yl)-acetic acid ST2304 (40)

3.6 g (0.0061 mol) of [(2R,11aS)-2-{[terz-butyl-(diphenyl)silyl]oxy}-5,11-dioxo-2,3,11,11a-tetrahydro-1H-pyrrole[2,1-c]-[1,4]benzo-diazepin-10(5H)-yl)-acetic acid terz-butyl ester (39) are suspended in 30 ml of CH₂Cl₂ and 25 ml of trifluoroacetic acid are added dropwise under stirring at 15° C. The temperature is allowed to rise to room temperature, and then the solution is left to stir for 45 minutes. The reaction solution is brought to dryness under reduced pressure with a mechanical pump; the residue is solubilised in ethyl ether from which it is precipitated by cooling, forming 2.6 g of a white solid (yield: 81%).

TLC: CHCl₃ 8/MeOH 2/CH₃COOH 0.1; RF: 0.51.

M.P.: 191-193° C.

E.A.: Theoretical:+0.6% H₂O (TG): C, 67.74; H, 6.13; N, 5.26;

Found: C, 67.12; H, 6.09; N, 5.28.

[α]²⁰_{D}+246.2; conc. 1% CHCl₃.

1H-NMR (300 MHz, CDCl₃): δ 1.03 (s, 9H), 2.05 (m, 1H), 2.75 (m, 1H), 3.50, 3.55 (2d, 1H), 3.75, 3.78 (2d, 1H), 4.25 (m, 1H), 4.20-4.57 (dd, 2H), 4.60 (m, 1H), 7.10 (d, 1H), 7.20-7.50 (m, 8H), 7.60 (m, 4H), 7.90 (d, 1H).

HPLC: Column: Inertsil ODS 3(5μ) 4.6×250 mm;

Mobile phase: CH₃CN/KH₂PO₄ 50 mM 70/30; pH 3 (H₂PO₄ 85%);

Flow rate: 1.0 ml/min, t=30° C.;

R.T.: 10.79 min.

The building block ST2393 (45), useful for the synthesis of the peptidomimetic compounds containing the β-turn mimetic Beta5, was synthesised according to Scheme 5, using the method described by M. R. Peña and J. K. Stille (J. Am. Chem. Soc.; 1989, 111, 5417-5424), using isatoic anhydride (41) and L-proline (42) as the starting products. The synthesis is described in Example 6 here below.

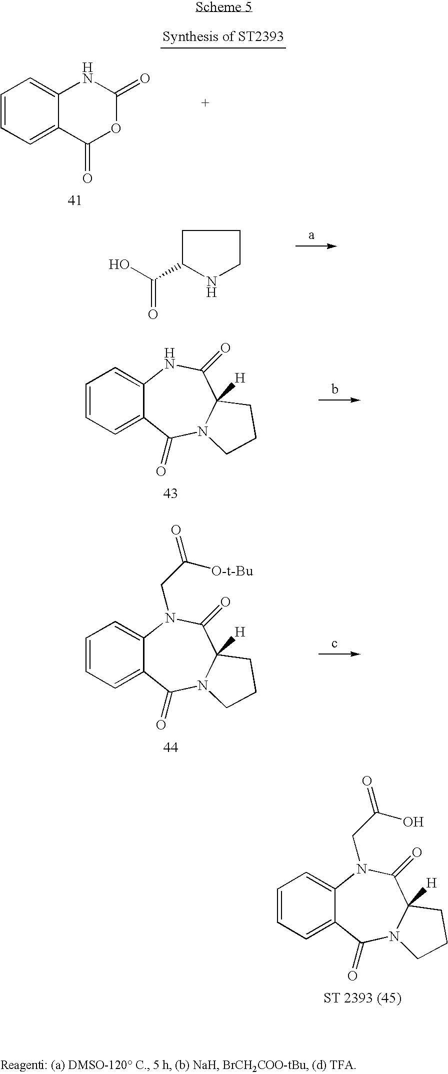

### EXAMPLE 6

### Preparation of [(11aS)-5,11-dioxo-2,3,11,11a-tetrahydro-1H-pyrrole[2,1-c][1,4]benzodiazepin-10(5H)-yl]-acetic acid ST 2393 (45)

### Preparation of the Intermediate (11aS)-2,3-dihydro-1H-pyrrole[2,1-c]-[1,4]benzodiazepine-5,11(10H,11aH)-dione (43)

2.67 g (0.0164 mol) of isatoic anhydride (41) are solubilised in 20 ml of anhydrous DMSO under an N₂ atmosphere and 1.72 g of L-proline (0.0149 mol) (42) are added under stirring. The solution is then heated to 120° C. under stirring for 2.5 hours, then cooled and poured into 130 ml of cold H₂O under stirring. The precipitate formed by the aqueous solution is filtered and washed again with cold water and oven-dried in vacuo for 3 hours. 2.6 g are obtained (yield: 80%).

TLC: AcOEt; RF: 0.5.

[α]²⁰_{D}:+528 (0.5% CH₃OH).

¹H-NMR (200 MHz, CDCl₃): δ 2.05 (m, 3H), 2.80 (m, 1H), 3.65 (m, 1H) 3.82 (m, 1H) 4.12 (d, 1H), 7.00 (d, 1H), 7.30 (t, 1H), 7.52 (t, 1H), 8.03 (d, 1H), 8.25 (sa, 1H).

### Preparation of the Intermediate [(11aS)-5,11-dioxo-2,3,11,11a-tetrahydro-1H-pyrrole[2,1-c][1,4]benzodiazepin-10(5H)-yl]-acetic acid terz-butyl ester (44)

518 mg (0.0127 mol) of 60% NaH are suspended in 5 ml of anhydrous THF after washing several times with THF. The suspension is cooled to −40° C. and 2.5 g (0.0115 mol) of (11aS)-2,3-dihydro-1H-pyrrole-[2,1-c][1,4]benzodiazepine-5,11-(10H,11aH)-dione (43) solubilised in 25 ml of anhydrous THF are added dropwise, maintaining the solution under stirring. After 45 minutes 1.87 ml (0.0127 mol) of terz-butyl-bromoacetate are added dropwise and the mixture is left to react under stirring for 2 hours, allowing the temperature to rise to room temperature. The suspension is then poured into 150 ml of H₂O and extraction is done twice with 100 ml of CH₂Cl₂, washing the pooled organic phases several times with H₂O and salt and anhydrifying with anhydrous Na₂SO₄. The solution is brought to dryness under reduced pressure, obtaining an oil, which, after washings with petroleum ether and drying with a mechanical pump, gives 3.7 g of an amorphous solid (yield: 97%).

TLC: n-hexane 1/AcOEt 1; RF: 0.4.

¹H-NMR: (200 MHz, CDCl₃): δ 1.50 (s, 9H), 2.05 (m, 3H), 2.68 (m, 1H), 3.60 (m, 1H), 3.83 (m, 1H), 4.15 (m, 1H), 4.15 (d, 1H), 4.59 (d, 1H), 7.18-7.40 (m, 2H), 7.55 (dt, 1H), 7.97 (dd, 1H).

### Preparation of [(11aS)-5,11-dioxo-2,3,11,11a-tetrahydro-1H-pyrrole-[2,1-c][1,4]benzodiazepin-10(5H)-yl]-acetic acid ST2393 (45)

3.5 g (0.01 mol) of [(11aS)-5,11-dioxo-2,3,11,11a-tetrahydro-1H-pyrrole[2,1-c][1,4]benzodiazepin-10(5H)-yl]-acetic acid terz-butyl ester (44) are solubilised in 30 ml of CH₂Cl₂ and 25 ml of trifluoroacetic acid are added dropwise at 10° C. under stirring. The temperature is allowed to rise to room temperature, and the solution is then stirred for 1.5 hours. The reaction solution is brought to dryness under reduced pressure with a mechanical pump; the residue is treated with ethyl ether, from which it solidifies, giving 2.65 g of a white solid after oven drying in vacuo for 2 hours (yield: 96%).

TLC: CHCl₃ 60/MeOH 40/H₂O 15/IprOH 10/CH₃COOH 15; RF: 0.8.

M.P.: 267-269° C.

E.A.: Theoretical:+1.9% H₂O (TG): C, 60.14; H, 5.26; N, 10.02;

Found: C, 60.31; H, 5.36; N, 9.70.

[α]²⁰_{D}:+408 (0.7% CHCl₃).

¹H-NMR (300 MHz, MeOD): δ 2.07 (m, 3H), 2.61 (m, 1H) 3.55 (m, 1H), 3.77 (m, 1H), 4.25 (m, 1H), 4.48 (d, 1H), 4.60 (d, 1H), 7.38 (m, 2H), 7.60 (m, 1H), 7.83 (m, 1H).

HPLC: Column: Symmetry C18 (3.5);

Mobile phase: KH₂PO₄ 50 mM/CH₃CN;

Gradient: from 100% to 30% KH₂PO₄ 50Mm;

Flow rate: 1.0 mL/min., r. temp.;

R.T.: 10.85 min.

The building block ST2590 (52), useful for synthesising the peptidomimetic compounds containing the β-turn mimetic Beta7,was synthesised according to Scheme 6 here below, starting from the commercial products (46) and (47). The synthesis of ST2590 is described in Example 7 here below:

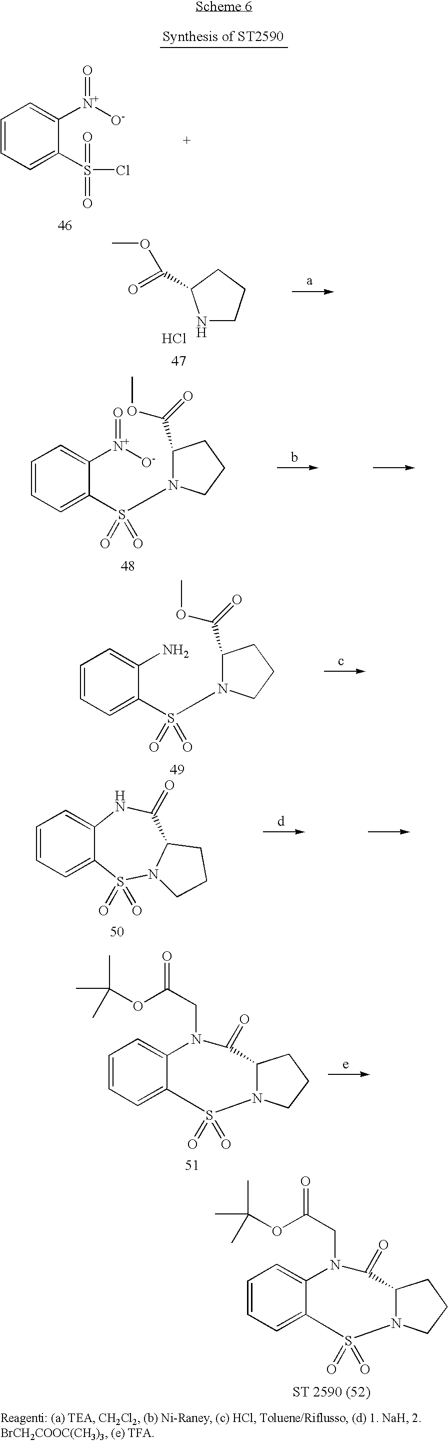

### EXAMPLE 7

### Preparation of [(11aS)-5,5-diossido-11-oxo-2,3,11,-11a-tetrahydro-pyrrole[1,2-b][1,2,5]benzothiadiazepin-10(1H)-yl]-acetic acid ST2590 (52)

### Preparation of the Intermediate 1-[(2-nitrophenyl)sulphonyl]-L-proline methylester (48)

5.0 g (30 mmol) of L-proline methylester (47) are suspended in 30 ml of CH₂Cl₂ and 8.5 ml (60 mmol) of triethylamine are added under stirring. 6.75 g (30 mmol) of 2-nitro-benzenesulphonyl chloride (46) in 30 ml di CH₂Cl₂ are added dropwise under N2 at 5° C. and the mixture is left to react under stirring for 1.5 hours with an N2 atmosphere. The reaction solution is washed twice with cold water, and the organic phase is separated and anhydrified on anhydrous Na₂SO₄ and brought to dryness in vacuo, obtaining an oily crude product which, after chromatography on silica with n-hexane/ethyl acetate 6:4 as the eluent, gives 3.5 g of product (yield: 37%).

TLC: n-hexane 1/AcOEt:1; R.F. 0.56.

¹H-NMR (200 MHz, CDCl₃): δ 1.90-2.20 (m, 3H), 2.20-2.45 (m, 1H), 3.45-3.85 (m, 1H), 3.70 (s, 3H), 4.65 (dd, 1H), 7.70 (m, 3H), 8.17 (m, 1H).

### Preparation of the Intermediate 1-[(2-aminophenyl)-sulfphonyl]-L-proline methylester (49)

3.95 g (12.5 mmol) of 1-[(2-nitrophenyl)-sulphonyl]-L-proline methylester (48) are solubilised in 55 ml of methanol, an excess of Ni-Raney is added as a 50% aqueous dispersion after activating it in methanol and the solution is left to stir for 2.5 hours. More activated Ni-Raney is added, and the solution is then left to stir for another 45 minutes and filtered on celite, washing with a mixture of ethyl acetate and methanol without bringing the Ni-Raney residue to dryness. The methanol filtering solutions are anhydrified on anhydrous Na₂SO₄ and brought to dryness in vacuo, giving 3.1 g of a sufficiently pure product (yield: 87%).

TLC: n-hexane 1/AcOEt 1; R.F. 0.45.

¹H-NMR (200 MHz, CDCl₃): δ 1.80-2.35 (m, 4H), 3.40 (t, 2H), 4.55 (dd, 1H), 5.05-5.40 (sa, 1H), 6.74 (t, 2H), 7.30 (t, 1H), 7.72 (d, 1H).

### Preparation of the Intermediate (11aS)-1,2,3,11a-tetrahydropyrrole-[1,2-b][1,2,5]benzothiadiazepin-11(10H)-one 5,5-dioxide (50)

3.1 g (10.9 mmol) of 1-[(2-aminophenyl)-sulphonyl]-L-proline methylester (49) are solubilised in 250 ml of toluene together with 50 mg of p-toluenesulphonic acid (0.268 mmol) and heated at reflux temperature for three days, eliminating the water by means of Dean-Stark. The solution is then washed twice with a saturated solution of NaHCO₃ and with water at neutral pH, anhydrified with anhydrous Na₂SO₄, filtered and brought to dryness under reduced pressure. The brown crude solid is crystallised hot with hexane and ethyl acetate, obtaining 1.1 g of product. The mother waters, brought to dryness under reduced pressure, are treated by flash chromatography using n-hexane/AcOEt 55:45 as the eluent, thus obtaining another 550 mg. A total of 1.65 g of product is thus obtained (yield: 60%).

TLC: n-hexane1/AcOEt 1; R.F. 0.25.

¹H-NMR (200 MHz, CDCl₃): δ 1.75-2.15 (m, 2H), 2.15-2.38 (m, 1H), 2.38-2.63 (m, 1H), 3.05 (q, 1H), 3.55 (q, 1H), 4.65 (t, 1H), 7.00-7.40 (m, 2H), 7.55 (t, 1H), 7.95 (d, 1H), 8.80 (sa, 1H).

### Preparation of the Intermediate [(11aS)-5,5-dioxido-11-oxo-2,3,11, 11a-tetrahydropyrrole[1,2-b][1,2,5]benzothiadiazepin-10(1H)-yl]acetic acid terz-butyl ester (51)

To a suspension of 200 mg of NaH (0.0087 mol) in 15 ml of anhydrous THF brought to −40° C. and under nitrogen flow is added a solution of 2.0 g of (11aS)-1,2,3,11a-tetrahydropyrrole[1,2-b][1,2,5]-benzothiadiazepin-11(10H)-one 5,5-dioxide (50) (0.0079 mol) in 25 ml of anhydrous THF. After 45 minutes at −40° C. 1.17 ml of terz-butyl-bromoacetate (0.0087 mol) are added, allowing the temperature to rise to 20° C. (r. temp.) and the solution is left to stir for 2 hours. The solution is diluted with cold water and extracted twice with CH₂Cl₂, pooling the organic extracts which are washed with water and salt. The organic solution is anhydrified with anhydrous Na₂SO₄, filtered and brought to dryness under reduced pressure. The product is then crystallised hot with hexane and ethyl acetate, obtaining 1.76 g of product (yield: 61%).

TLC: n-hexane/AcOEt 1:1; R.F. 0.55.

¹H-NMR (200 MHz, CDCl₃): δ 2.52 e 2.58 (2s, 9H), 2.80-2.22 (m, 3H), 2.45 (m, 1H), 3.45 (m, 2H), 3.88 (t, 1H), 4.05 (d, 1H), 4.70 (d, 1H), 7.45 (t, 2H), 7.68 (t, 1H), 8.03 (d, 1H).

### Preparation of [(11aS)-5,5-dioxido-11-oxo-2,3,11,11a-tetra-hydro-pyrrole[1,2-b][1,2,5]benzothiadiazepin-10(1H)-yl])-acetic acid ST2590 (52)

To a solution of 1.75 g (0.00478 mol) of [(11aS)-5,5-dioxido-11-oxo-2, 3,11,11a-tetrahydropyrrole[1,2-b][1,2,5]benzothiadiazepin-10(1H)-yl]acetic acid terz-butyl ester (51) in 20 ml of CH₂Cl₂ brought to 0° C. are added dropwise 13 ml of TFA, leaving the solution to stir at room temperature for 2 hours.

After the TLC control (Silica, CHCl₃ 9/MeOH 1) the solvent is evaporated, the residue is taken up with CH₂Cl₂ and the organic phase is treated with a saturated solution of Na₂CO₃ which is separated.

This is then washed with ethyl ether and treated with a concentrated HCl solution. A product is precipitated from the solution, which, when extracted with CH₂Cl₂, anhydrified with Na₂SO₄ and brought to dryness on the rotavapor, gives 1.1 g of 98% pure final product (yield: 74%).

M.P.: 149-152° C.

[α]_{D}²⁰:+210.1; conc. 0.47% Me-OH.

HPLC: Column: Symmetry C18 (3.5μ) 4.6×150 mm;

Mobile phase: KH₂PO₄ 50 mM pH3/CH₃CN 80/20;

Flow rate: 1.0 ml/min; r. temp.;

R.T.: 10.51 min.

¹H-NMR (300 MHz, CDCl₃): δ 1.8-2.2 (3H, m, CH₂CH₂); 2.4-2.6 (1H, m, CH₂CH₂);3.4-3.6 (2H, m, CH₂N); 3.8-3.9 (1H, m, CHN); 4.25 and 4.75 (2H, 2d, CH₂COOH); 5.6-6.6 (1H, bs, COOH); 7.45 (2H, t, Ar); 7.7 (1H, t, Ar); 8.0 (1H, d, Ar).

E.A. (C₁₄H₁₄N₂O₄): Theoretical: C, 50.31; H, 4.54; N, 9.02; S, 10.33;

Found: C, 50.03; H, 4.45; N, 8.78; S, 10.13.

The building block ST2610 (64), useful for synthesising the peptidomimetic compounds containing the β-turn mimetic Beta8, was synthesised according to Scheme 7 here below, up to intermediate 61 using the method described by Ehab M. Khalil and co-workers., J. Med. Chem.; 1999, 42, 628-637, and then using the method described in Example 8 here below.

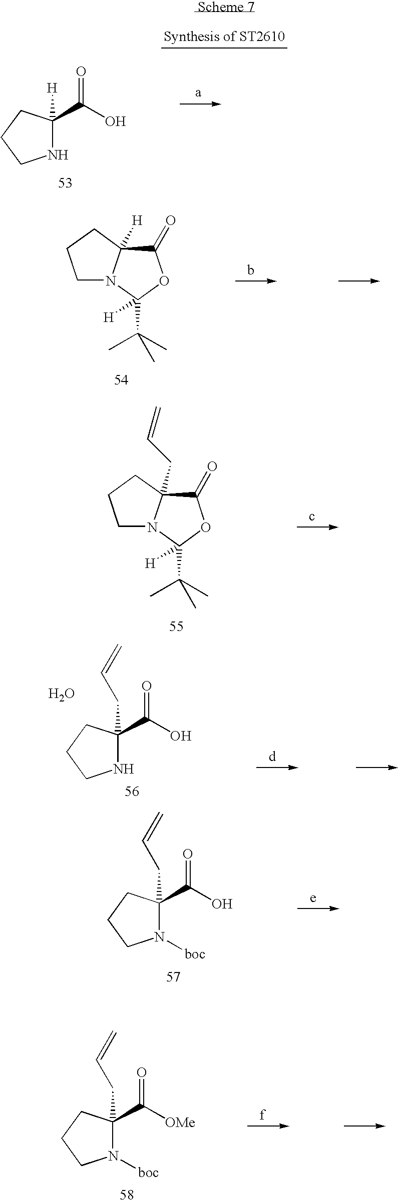

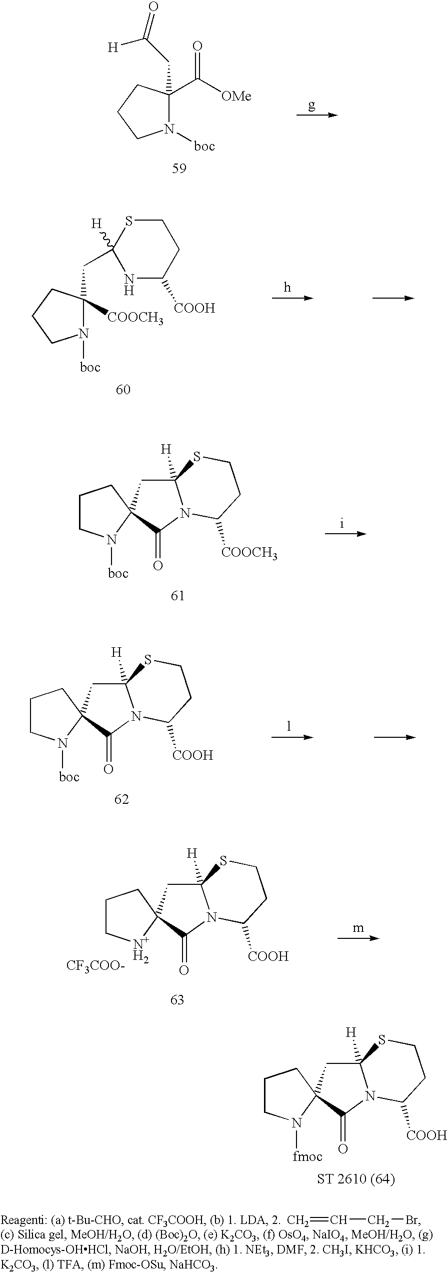

### EXAMPLE 8

### Preparation of (2R,4′R,8a′R)-1-[(9H-fluoren-9-yl-methoxy)carbonyl]-6′-oxotetrahydro-2′H-spiro[pyrrolidine-2,7′-pyrrole-[2,1-b][1,3]thiazine]-4′-carboxylic acid ST2610 (64)

### Preparation of the Intermediate (2R,4′R,8a′R)-1-(terz-butoxy-carbonyl)-6′-oxotetrahydro-2′H-spiro [pyrrolidine-2,7′-pyrrole[2,1-b]-[1,3]-thiazine]-4′-carboxylic acid (62)

2.55 g (0.0069 mol) of (2R,4′R,8a′R)-6′-oxotetrahydro-1H,2′H-spiro[-pyrrolidine-2,7′-pyrrole[2,1-b][1,3]thiazine]-1,4′-dicarboxylic acid 1-terz-butyl 4′-methylester (61) are dissolved in 45 ml of MeOH and 45 ml of H₂O; to the solution are added 1.9 g (0.0138 mol) of K₂CO₃. The solution is held under stirring at room temperature for 20 hours, and is then treated by lowing the pH to 5 and bringing it to dryness under reduced pressure. The residue obtained is taken up with H₂O, brought to pH 2-3 and extracted with CHCl₃. The organic phase is anhydrified on anhydrous Na₂SO₄ followed by evaporation to dryness. The pitchy mass is crystallised with ethyl ether, obtaining 2.4 g of a filterable white solid (yield: 97%).

TLC: CHCl₃ 8/MeOH 2/CH₃COOH 0.1; RF: 0.65;

¹H-NMR (300 MHz, CDCl₃): δ 1.40, 1.45 (2d, 9H), 2.70-2.05 (m, 4H), 2.00-2.30 (m, 1H), 2.25-2.55 (m, 1H), 2.55-2.80 (m, 1H), 2.75-3.15 (m, 1H), 3.50 (m, 2H), 3.70 (t, 2H), 4.98 (dd, 1H), 5.10 (d, 1H).

### Preparation of the Intermediate (2R,4′R,8a′R)-4′-carboxy-6′-oxotetrahydro-2′H-spiro[pyrrolidine-2,7′-pyrrole[2,1-b][1,3]thiazine]trifluoroacetate (63)

2.1 g (0.0058 mol) of (2R,4′R,8a′R)-1-(terz-butoxycarbonyl)-6′-oxotetrahydro-2′H-spiro[pyrrolidine-2,7′-pyrrole[2,1-b][1,3]thiazine]-4′-carboxylic acid (62) are dissolved in 45 ml of trifluoroacetic acid. The mixture is held under stirring at room temperature for 3 hours and evaporated to dryness at 30° C. under reduced pressure, The residue obtained is solubilised in a small amount of CHCl₃, precipitated with ethyl ether and the solid formed is filtered rapidly. This operation is performed twice, obtaining 1.55 g of a very hygroscopic white solid (yield; 74%).

TLC: (CHCl₃ 60/MeOH 40/H₂O 15/IsoPrOH 10/AcOH 15); RF: 0.68.

¹H-NMR (300 MHz, D₂O): δ 1.75-2.00 (m, 1H), 2.00-2.38 (m, 5H), 2.45 (dd, 1H), 2.70 (dt, 1H), 2.77-3.05 (m, 2H), 3.25-3.55 (m, 2H), 3.96 (d, 1H), 5.18 (d, 1H).

### Preparation of (2R,4′R,8a′R)-1-[(9H-fluoren-9-yl-methoxy)carbonyl]-6′-oxotetrahydro-2′H-spiro[pyrrolidine-2,7′-pyrrole[2,1-b][1,3]thiazine]-4′-carboxylic acid ST2610 (64)

1.48 g (0.004 mol) of (2R,4′R,8a′R)-4′-carboxy-6′-oxotetrahydro-2′H-spiro[pyrrolidin-2,7′-pyrrole[2,1-b]-[1,3]thiazine]trifluoroacetate (63) are solubilised in 50 ml of H₂O. 0.67 g (0.008 mol) of NaHCO₃ are added to the solution and then 1.42 g (0.0042 mol) of Fmoc-N—OSu dissolved in 75 ml of acetone. The reaction solution is left to stir at room temperature for 20 hours, and then the acetone is eliminated under reduced pressure, H₂O is added and the solution is washed with Et₂O. The aqueous solution is brought to pH 3 with HCl 2N and extraction is done with CHCl₃. The organic phase is dried on anhydrous Na₂SO₄ and evaporation is performed, obtaining 1.9 g of a glassy white solid (yield: 98%).

M.P.: 108°-115° C.

[α]_{D}²⁰: +70.1.

E.A.: (C₂₆H₂₆N₂O₅S). Theoretical: 7.47% H₂O: C, 60.38; H, 5.90; N, 5.41; S, 6.20;

Found: C, 56.77; H, 4.85; N, 5.06; S, 5.26.

¹H-NMR (300 MHz, CDCl₃): δ 1.85 (m, 1H), 1.94 (dd, 1H), 2.05 (m, 1H), 2.15 (m, 1H), 2.42 (m, 1H), 2.67 (d, 1H), 2.82 (m, 2H), 3.00 (m, 1H), 3.58 (dd, 2H), 4.20 (t, 1H).

HPLC: Column: Symmetry C18 (5μ) 3.9×150 mm;

Mobile phase: KH₂PO₄ 50 mM/CH₃CN 65/35;

Flow rate: 1.0 ml/min; r. temp.;

R.T.: 12.19 min.

The building block ST2775 (78), useful for synthesising the peptidomimetic compounds containing the β-turn mimetic Beta9, was synthesised according to Scheme 8, up to intermediate 75 which was synthesised using the method described by Ehab M. Khalil and co-workers, J. Med. Chem.; 1999, 42, 628-637, and then using the method described in Example 9 here below.

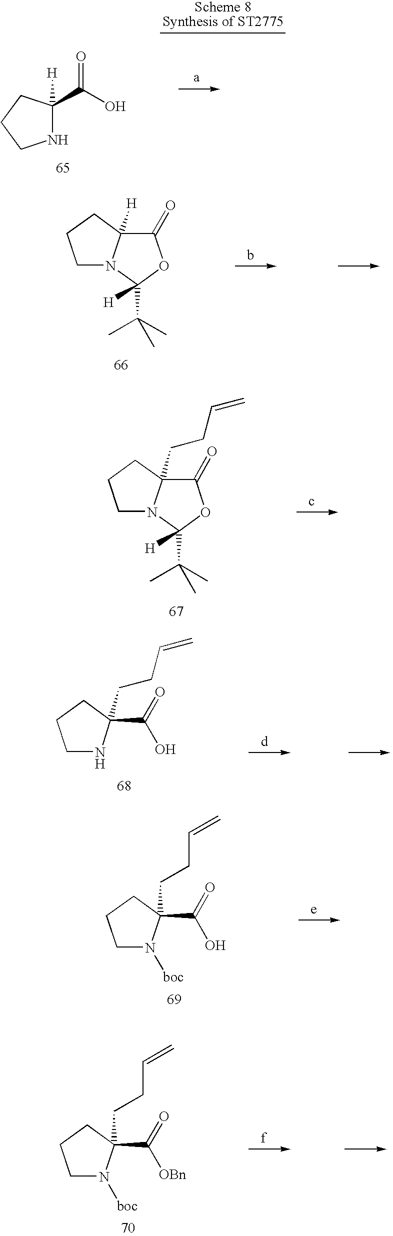

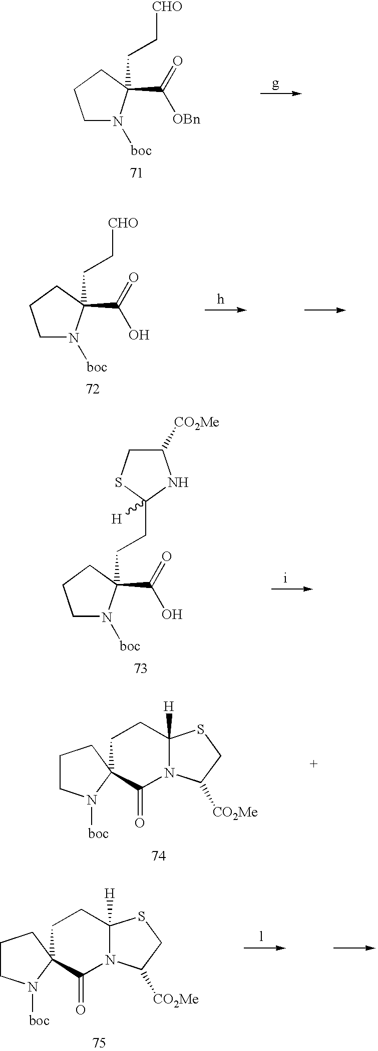

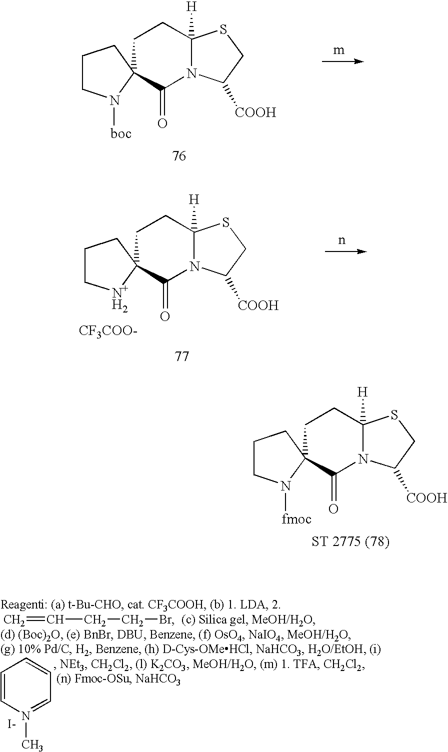

### EXAMPLE 9

### Preparation of (2R,3′S,8a′R)-1-[(9H-fluoren-9-yl-methoxy)carbonyl]-5′-oxotetrahydro-7′H-spiro[pyrrolidine-2,6′-[1,3]-thiazole[3,2-α]pyridine]-3′-carboxylic acid ST2775 (78)

### Preparation of the Intermediate (2R, 3′S, 8a′R)-1-(terz-butoxy-carbonyl)-5′-oxotetrahydro-7′H-spiro[pyrrolidine-2,6′-[1,3]thiazole[3,2-α]-pyridine]-3′-carboxylic acid (76)

1.5 g (0.004 mol) of (2R,3′S,8a′S)-5′-oxotetrahydro-1H,7′H-spiro[pyrrolidine-2,6′-[1,3]thiazole[3,2-α]pyridine]-1,3′-dicarboxylic acid 1-terz butyl 3′-methylester (75) are dissolved in 80 ml of MeOH and 50 ml of H₂O. To the solution are added 1.1 g (0.008 mol) of K₂CO₃ and the reaction mixture is left to stir at room temperature for 20 hours.

The reaction solution is treated by lowering the pH to 5 with HCl 2N and then bringing it to dryness under reduced pressure. The residue obtained is taken up with H₂O, brought to pH 2-3 and extraction is performed with CHCl₃. The organic phase is anhydrified on anhydrous Na₂SO₄, followed by evaporation to dryness, obtaining 1.4 g of a glassy white solid (yield: 100%).

TLC: CHCl₃ 8/MeOH 2/CH₃COOH 0.1; RF: 0.7.

¹H-NMR (300 MHz, CDCl₃): δ 1.45, 1.50 (2s, 9H), 1.60-2.18 (m, 6H), 2.20-2.50 (m, 2H), 3.20-3.80 (m, 4H), 4.95 (dd, 1H), 4.38 (dd, 1H), 7.35 (sa, 1H).

### Preparation of the Intermediate (2R,3′S,8a′R)-3′-carboxy-5′-oxo-tetrahydro-7′H-spiro[pyrrolidine-2,6′-[1,3]thiazole[3,2-α]pyridine]trifluoroacetate (77)

1.4 g (0.004 mol) of (2R,3′S,8a′R)-1-(terz-butoxycarbonyl)-5′-oxotetrahydro-7′H-spiro[pyrrolidine-2,6′-[1,3]thiazole-[3,2-α]pyridine]-3′-carboxylic acid (76) are dissolved in 25 ml of CH₂Cl₂ and 25 ml of trifluoroacetic acid. The mixture is stirred at room temperature; after 2 hours evaporation is performed at 30° C. The residue obtained is solubilised in a small amount of CHCl₃, precipitated with Et₂O and the precipitate formed is filtered rapidly. This operation is repeated twice. 1.07 g of a very hygroscopic white solid are obtained (yield: 70%).

TLC: CHCl₃ 60/MeOH 40/H₂O 15/IPrOH 10/AcOH 15; RF: 0.5.

¹H-NMR (300 MHz, D₂O): δ 1.70-2.50 (m, 8H), 3.15 (dd, 1H), 3.22-3.60 (m, 3H), 4.60-5.00 (m, 2H).

### Preparation of (2R,3′S,8a′R)-1-[(9H-fluoren-9-yl-methoxy) carbonyl]-5′-oxotetrahydro-7′H-spiro[pyrrolidine-2,6′-[1,3]thiazole[3,2-α]pyridine]-3′-carboxylic acid ST2775 (78)

1.00 g (00027 mol) of (2R,3′S, 8a′R)-3′-carboxy-5′-oxotetrahydro-7′H-spiro[pyrrolidine-2,6′-[1,3]thiazole[3,2-α]pyridine]trifluoroacetate (77) is dissolved in 50 ml of water. 0.45 g (0.0054 mol) of NaHCO₃ and after 5 minutes 0.94 g. (0.0028 mol) of Fmoc-N—OSu dissolved in 75 ml of acetone are added to the solution obtained. More acetone is added to the suspension formed until a solution is obtained, which is left to stir at room temperature. After 24 hours, the acetone is evaporated, the water is restored and washing is done with Et₂O. The mixture is then brought to pH 3 with HCl 2 N and the aqueous phase is extracted with CHCl₃. The organic phase is anhydrified with anhydrous Na₂SO₄ and brought to dryness under reduced pressure, obtaining 1.2 g of a glassy white solid (yield: 90%).

M.P.: 95°-100° C.

E.A.: (C₂₆H₂₆N₂O₅S).

Theoretical with 3.76% H₂O: C, 62.80; H, 5.69; N, 5.63; S, 6.44.

Found: C, 57.27; H, 4.89; N, 4.97; S, 5.25.

[α]²⁰_{D}:+108.6; conc. 0.5% in MeOH.

TLC: CHCl₃ 8/MeOH 2; RF: 0.64.

¹H-NMR (CDCl3 300 MHz): δ 1.60-2.20 (m, 5H), 2.25-2.45 (m, 2H), 2.80, 3.05 (2dd, 1H), 3.30-3.50 (m, 2H), 3.50-3.70 (m, 2H), 4.07, 4.17-4.35 (s, m, 2H), 4.37-4.50 (m, 1H), 4.73, 4.92 (t, dd, 1H), 5.35, 5.65 (2dd, 1H), 5.00-6.40 (sa, 1H), 7.22-7.45 (m, 4H), 7.50-7.65 (m, 2H), 7.65-7.80 (m, 2H).

HPLC: Column: Symmetry C18 (5μ) 3.9×150 mm;

Mobile phase: KH₂PO₄ 50 mM/CH₃CN 60/40;

Flow rate: 1.0 ml/min. r. temp.;

R.T.: 7.38 min;

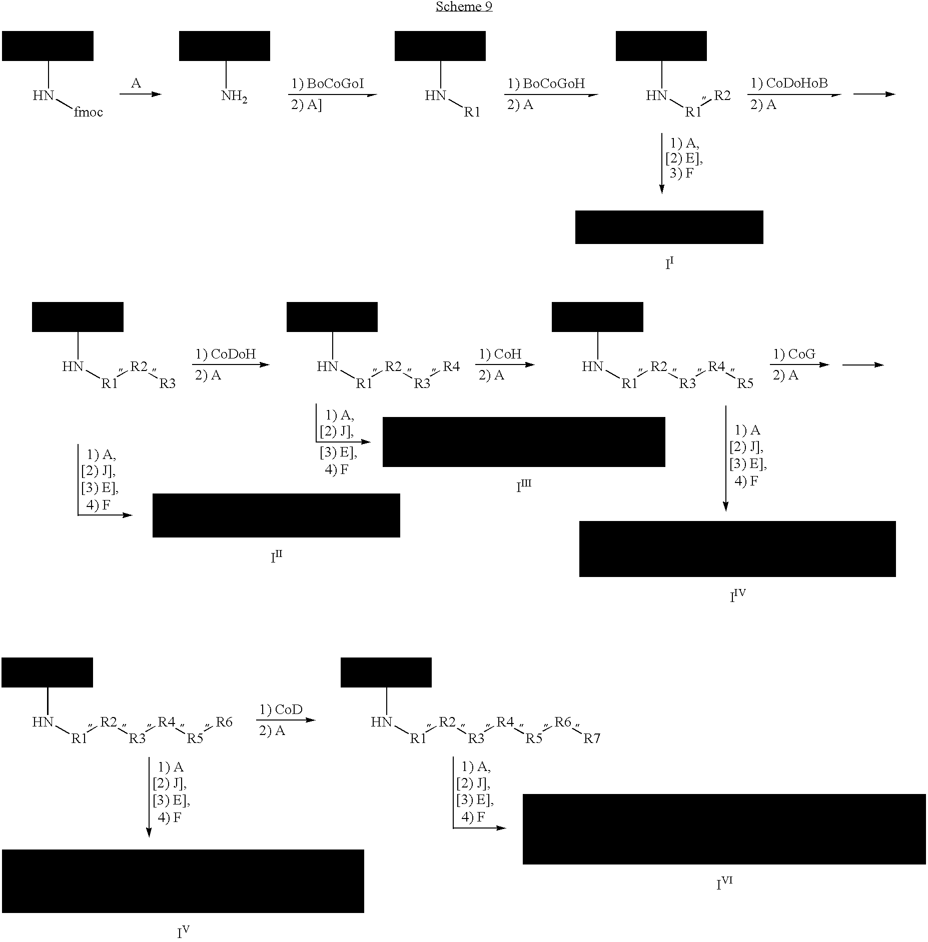

All the molecules were synthesised on a polymer support consisting of an aminomethyl NovaGel™ with a modified Rink linker (0.74 mmol/g) and the peptidomimetic sequences were synthesised with protocols based on Fmoc chemistry. The reactions were normally carried out in DMF, using HOBt-TBTU as activating species in the acylation reactions and DIPEA as a proton scavenger. Where a more vigorous activation of the carboxylic function was necessary, for example, in the acylation of aromatic amines, the reaction was con-ducted at 70° C. or by converting the carboxylic function of the Fmoc-protected acylating species into the corresponding acylic chloride^{[1]}, with thionyl chloride (SOCl₂). In this case, the reaction was conducted in tetrahydrofurane at 70° C. using DIPEA or collidine as proton scavengers.

The protected natural amino acids, the solvents and the reagents used in the synthesis were purchased from Chem-Impex; the substituted aminobenzoic acids (spacers) were purchased from Sigma-Aldrich and were protected with fluorenylmethyloxycarbonyl chloride (Fmoc-Cl), as described in the literature.

The LC/MS and MS-inf. analyses of the synthesis intermediates and reaction products were carried out on a Thermofinnigan LCQ-Duo Mass Spectrometry System using an H₂O-acetonitrile gradient system (solvent B) with pH buffered with 0.1% TFA for the HPLC-RP runs,

Gradient 1

Time

| | 0 | 2 | 13 | 15 | 18 | 20 |
|---|---|---|---|---|---|---|
| % B | 5 | 5 | 60 | 95 | 95 | 5 |

Gradient 2

Time

| | 0 | 5 | 45 | 48 | 50 | 52 |
|---|---|---|---|---|---|---|
| % B | 5 | 5 | 60 | 95 | 95 | 5 |

in Luna (Phenomenex) C18 columns; 50×2 mm; 3 μm, with a flow rate of 0.5 mL/min (Gradient 1) or Jupiter (Phenomenex) C18 columns; 250×4.6 mm; 5 μm, with a flow rate of 1.0 mL/min (Gradient 2) and all are understood to be developed on material obtained by treatment of a sample of dry resin (5-10 mg) with the cleavage mixture consisting of TFA/H₂O/Tis/(95:2.5:2.5) (300 μL) for 3 hours.

The semipreparative HPLC purifications were done on a Jupiter-Proteo (Phenomenex) C column; 250×21.2 mm; 10 μm; 90 Å, using an H₂O-MeCN-TFA (97:3:0.1) gradient system [solvent A] and MeCN:H₂O:TFA (80:20:0.1) [solvent B]

| | Time | | | | | | |
|---|---|---|---|---|---|---|---|
| | 3 | 20 | 10 | 10 | 10 | 5 | 3 |
| % B | 8 | 30 | 40 | 60 | 98 | 98 | 8. |

Abbreviations
AAA amino acid (generic indication)
Ac₂O acetic anhydride
DMF dimethylformamide
DIPEA diisopropylethylamine
DIPEA_{abs} pure diisopropylethylamine
Fmoc 9-fluorenylmethyloxycarbonyl
HOBt 1-hydroxy-1-H-benzotriazole
MeCN acetonitrile
min minutes
TBTU 2-(1H-benzotriazol-1-yl)-1,1,3,3-tetramethyluronium tetrafluoroborate
THF tetrahydrofurane
TFA trifluoroacetic acid
Tis triisopropylsilane
   Typical Reaction Procedures (See Scheme 9)
   Typical Procedure A—Removal of Fmoc from the Amine Function of the Linker of the Rink Amide MBHA Resin or from the Peptidemimetic Sequence Anchored to the Resin.

In a polypropylene reactor equipped with a cap and with a porous frit and drainage cock on the bottom, the swelling of the resin was done with methylene chloride (15 mL/g of resin) and dimethylformamide (15 mL/g of resin). The resin was then treated with a 25% solution of piperidine in dimethylformamide (15 mL/g of resin) for removal of the Fmoc protecting group from the amine function of the linker or from the peptidomimetic sequence anchored to the resin and growing. The mixture was stirred for 20 minutes. After the time indicated, the deprotection mixture was drained off and the resin was washed with dimethylformamide (15 mL/g of resin, repeated 5 times for 5 minutes each).

Typical Procedure B—Loading of the First Amino Acid or β-Turn Mimetic on Rink Amide MBHA.

In a polypropylene reactor equipped with a cap and with a porous frit and drainage cock on the bottom, the free amine function of the resin linker, obtained as described in Section A, was treated with the acylating species (5 equivalents relative to the resin) for the loading of the first amino acid or β-turn mimetic using the standard procedure for the formation of the peptide bond described in Typical Procedure C. The reaction trend was monitored by means of the Kaiser test for non-reacted amines. With a positive Kaiser test, the resin was drained off from the reaction mixture and washed with dimethylformamide (15 mL/g of resin, repeated 5 times for 5 minutes of each washing).

Typical Procedure C—Loading of Amino Acids or Hydrophobic Spacers on Peptidyl-Aminoacyl-Resin or β-Turn Mimetic-Resin.

In a polypropylene reactor equipped with a cap and with a porous frit and drainage cock on the bottom, the Fmoc-deprotected peptidomimetic sequence anchored to the resin as described in Typical Procedure B, was treated with the acylating species (5 equivalents relative to the resin), dissolved in a solution of HOBt and TBTU 0.5 M in dimethylformamide (5 equivalents relative to the resin) and DIPEA 1.0 M in dimethylformamide (10 equivalents relative to the resin). The mixture was stirred and wherever possible, the coupling trend was monitored by means of the Kaiser test. Within not more than 120 minutes, the resin was drained off from the reaction mixture and washed with dimethylformamide (15 mL/g of resin, repeated 5 times for 5 minutes each).

Typical Procedure D—Coupling of Amino Acids or Argininomimetics to the Aromatic Amine Function of Hydrophobic Spacers.

In a glass reactor with a screw cap, to the peptidomimetic sequence anchored to the resin were added the acyl chloride of the parent Fmoc-amino acid or arginino-mimetic (5 equivalents relative to the resin, for the preparation see down the typical procedure) and the proton scavenger DIPEA (or collidine) (500 μL) dissolved in tetrahydrofurane. The mixture was stirred for 2 hours at 70° C. With a positive test result, the resin was drained off from the reaction mixture and washed with dimethylformamide (15 mL/g of resin, repeated 5 times for 5 minutes each).

Preparation of the argininomimetic Acyl Chloride. Typical Procedure: In a glass reactor thionyl chloride (5 mL) was added to a suspension of arginino-mimetic acid or Fmoc protected arginino-mimetic acid in ethyl ether (16 mmol in 20 mL) under magnetic stirring. The suspension obtained was heated up to reflux conditions (75-80° C.) for 2 hours. During this phase, the mixture became limpid. When the transformation was complete (IPC: TLC on silica gel 60 F₂₅₄ Merck plates, of the methanolysis product, eluent N-hexane-AcOEt 70:30), the reaction mixture was cooled to ambient temperature and the solution evaporated. The oily residue, co-evaporated with N-hexane (4×50 mL) and dried in high vacuo, ultimately yielded the expected product.

Typical Procedure E—Acetylation of the N-Terminal Amine Function of the Peptidomimetic Anchored to the Resin.

On completing the synthesis of the peptidomimetic and removing the Fmoc protecting group as described in Typical Procedure A, in a polypropylene reactor equipped with a cap and with a porous frit and drainage cock on the bottom, the sequence anchored to the resin was treated with a mixture of Ac₂O/DIPEA/DMF [15:45:40] (10 mL/g of resin, repeated twice for 30 minutes each) for the acetylation of the α-amine function of the N-terminal amino acid of the peptidomimetic sequence. The acetylation trend was monitored by means of the Kaiser test. With a positive test result, the resin was drained off from the reaction mixture, washed with dimethylformamide (15 mL/g of resin, repeated 5 times for 5 minutes each), dichloromethane (15 mL/g of resin, repeated 3 times for 5 minutes each), ethyl ether (15 mL/g of resin) and dried by means of a gaseous nitrogen flow passage.

Typical Procedure F—Detachment of the Peptidomimetic Sequence from the Rink Amide MBHA Resin.

In a polypropylene reactor equipped with a cap and with a porous frit and drainage cock on the bottom, the resin, after washing with ethyl ether and nitrogen flow, was treated with a mixture of TFA-Tis-H₂O [95:2.5:2.5] (10 mL/g of resin) for detachment of the peptidomimetic. The mixture was stirred for 2 hours. After the time indicated the mixture was filtered from the resin and ethyl ether (15 mL) was added to the filtrate. The solution obtained was cooled for 12 hours at −20° C. Where a precipitate separated from the solution, the mixture was centrifuged to recover the solid. In the absence of the latter, the solution was evaporated under reduced pressure to recover the residue.

Typical Procedure G—Formation of Azaglycine on Resin.

For this procedure the protocol used was that described by Kessler in the article in J. Org. Chem.; 1999, 64, 7388-7394 using Fmoc-protected Rink amide MBHA after treating it according to the description in Typical Procedure A, instead of the resin described in the article:

In a polypropylene reactor equipped with a cap and with a porous frit and drainage cock on the bottom, the Fmoc deprotected Rink amide MBHA resin or the Fmoc-deprotected peptidomimetic sequence anchored to the resin prepared as described in Typical Procedure B was washed with anhydrous CH₂Cl₂ (3×1 mL) and a solution of 5-(9H-fluoren-9-ylmethoxy)-1,3,4-oxadiazol-2(3H)-one (5 equivalents relative to the resin) in anhydrous CH₂Cl₂ (1 mL) was added. The reaction mixture was shaken at room temperature for 90 min., the resin was drained off from the reaction mixture and washed with anhydrous CH₂Cl₂ (3×1 mL) and dimethylformamide (3×1 mL).

Typical Procedure H—Formation of Azavaline and Azaleucine on Resin.

For this procedure the protocol used was that described by Kessler in the article in J. Org. Chem.; 1999, 64, 7388-7394 for the formation of aza-alanine on resin 2-(chlorocarbonyl)-1-Fmoc-2-isopropyl-hydrazine or 2-(chlorocarbonyl)-1-Fmoc-2-isobutyl-hydrazine instead of 2-(chlorocarbonyl)-1-Fmoc-2-methyl-hydrazine and using Fmoc-protected Rink amide MBHA resin instead of the resin described in the article, after treating it according to the description in Typical Procedure A:

In a polypropylene reactor equipped with a cap and with a porous frit and drainage cock on the bottom, the Fmoc deprotected Rink amide MBHA resin or the Fmoc-deprotected peptidomimetic sequence anchored to the resin prepared as described in Typical Procedure B was washed with anhydrous dimethylformamide (3×1 mL) and a solution of 2-(chlorocarbonyl)-1-Fmoc-2-isopropyl-hydrazine or 2-(chlorocarbonyl)-1-Fmoc-2-isobutyl-hydrazine (5 equivalents relative to the resin) and 47 μL of DIPEA in 1 mL of anhydrous di-methylformamide was added. The reaction mixture was shaken at room temperature for 15 h, the resin was drained off from the reaction mixture and washed with anhydrous dimethylformamide (3×1 mL).

Typical Procedure I—Preparation of the Arginino-Mimetic AM8. Synthesis of the Guanidylic Group by Fmoc-Isothiocyanate on Rink Amide MBHA Resin.

In a polypropylene reactor equipped with a cap and with a porous frit and drainage cock on the bottom, Fmoc-isothiocyanate (10 equivalents relative to the resin) dissolved in dimethylformamide (4.0 mL/g) was added to Rink amide MBHA resin treated as de-scribed in Typical Procedure A. The mixture was stirred for 12 hours. After this time, the resin was drained off, washed with di-methyl-formamide (5 mL×5) and treated with a solution of CH₃I (10 equivalents relative to the resin) and DIPEA (30 equivalents relative to the resin) in dimethylformamide (10 mL). The mixture was stirred for 2 hours at room temperature, and then drained and washed with dimethylformamide (5 mL×5). To this material was added a solution of 1,4-diaminobutane (10 equivalents relative to the resin) in dimethylformamide (10 mL). The mixture was stirred for 12 hours at room temperature. After this time, the resin was drained off and washed with dimethylformamide (5×5 mL).

Typical Procedure J—Guanidinylation of the Terminal Amine Function of a Peptidomimetic Sequence Anchored to the Rink Amide MBHA Resin.

In a polypropylene reactor equipped with a cap and with a porous frit and drainage cock on the bottom, the resin to which the Fmoc-free peptidomimetic sequence was anchored was treated by guanidinylation of the N-terminal amine function, with a solution of 1,3-di-Boc-2-(trifluoromethylsulphonyl)guanidine (3.0 equivalents) in dichloromethane (15 mL/g). The mixture was stirred for 24 hours. With a positive test result, the resin was drained off the reaction mixture, washed with dichloromethane (15 mL/g of resin, repeated 5 times for 5 minutes each) and ethyl ether (15 mL/g of resin) and dried by gaseous nitrogen flow.

### EXAMPLE 10

### Preparation of ST2565 [Ac-thr-gly-pro-leu-val-asp-arg-NH₂, MW=797.4].

In a polypropylene reactor equipped with a cap and with a porous frit and drainage cock on the bottom, Fmoc-(D)Arg(pbf)-OH (250 μmol) was added to Rink amide MBHA (66 mg,˜50 μmol) treated as described in Typical Procedure A, for the loading of the first amino acid as described above. The mixture was stirred for 45 minutes. After the time indicated, the resin was washed with dimethyl-formamide (2 mL×5) and treated for the removal of the Fmoc (Typical Procedure A). The synthesis cycle described was repeated for the loading of the following amino acids after the first. To the growing peptidomimetic were added in sequence Fmoc-(D)Asp(Ot-Bu)-OH (250 μmol), Fmoc-(D)Val-OH (250 μmol), Fmoc-(D)Leu-OH (250 μmol), Fmoc-(D)Pro-OH (250 μmol), Fmoc-Gly-OH (250 μmol), and Fmoc-(D)Thr(OtBu)-OH (250 μmol). The couplings were monitored by means of the Kaiser test. On completing the sequence and removing the Fmoc as described, the peptidomimetic was treated with a solution of Ac₂O/DIPEA/DMF [15:45:40] (2 mL×2×30 min) for the acetylation of the α-amine function of the last amino acid, as described above. The acetylation trend was monitored by means of the Kaiser test. With a positive test result, the resin was washed with dimethylformamide (2 mL×5), dichloromethane (2 mL×3), and ethyl ether (2 mL) and dried with a gaseous nitrogen flow.

The dry resin was treated by cleavage of the peptide with a mixture of TFA-Tis-H₂O [95:2.5:2.5] (1 mL for 2 hours) as described above. After the time indicated, the mixture was filtered and ethyl ether (15 mL) was added to the filtrate. As a result of this operation a precipitate separated from the solution. The suspension was kept at −20° C. for 12 hours and then centrifuged. The solid obtained was dissolved in a solution of H₂O/acetonitrile/TFA [50:50:0.1] (5 mL) and liophilised, yielding the expected peptide [13.5 mg; LC (Gradient 1): retention time=5.70 minutes; Ms: (m+1)=within the given range].

### EXAMPLE 11

### Preparation of ST2792 [PAM9-SP02-Beta2-Thr-NH₂, MW=552.1]

In a polypropylene reactor equipped with a cap and with a porous frit and drainage cock on the bottom, Fmoc-Thr(OtBu)-OH (250 μmol) was added to the Rink amide MBHA resin (66 mg,˜50 μmol) treated as described in Typical Procedure A, for the loading of the first amino acid as described in Typical Procedures B and C. The mixture was stirred for 45 minutes. After this time, the resin was drained off from the reaction mixture, washed with dimethyl-formamide (2 mL×5) and treated for the removal of the Fmoc, Typical Procedure A.

The synthesis cycle described was repeated for the loading of Fmoc-[Beta2]-OH (250 μmol) and Fmoc-SP02-OH (250 μmol). After removing the Fmoc, PAM9-Cl (250 μmol) was added to the peptidemimetic sequence obtained, as described in Typical Procedure D, using DIPEA as a scavenger in the coupling reaction of PAM9-Cl with the amine function of SP02. The mixture was stirred for 2 hours at 70° C. With a positive test result, the resin was drained off from the reaction mixture, washed with dimethylformamide (2 mL×5), di-chloromethane (2 mL×3), and ethyl ether (2 mL×3) and dried by means of gaseous nitrogen flow. Finally, the dry resin was treated by cleavage of the peptidomimetic as described in Typical Procedure F. The cleavage product from the resin, purified by HPLC-RP and liophilised, ultimately yielded the expected molecule [9.1 mg; LC (Gradient 1): retention time=5.55 minutes; Ms: (m+1) within the given range.

### EXAMPLE 12

### Preparation of ST2864 [Ac-Gly-Pro-SP30-Arg-NH₂, MW=502.6].

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [19.0 mg; LC (Gradient 2): retention time=17.31 minutes; Ms: (m+1) within the given range].

### EXAMPLE 13

### Preparation of ST2794 [PAM9-Asp-Val-Val-Beta2-NH₂, MW=615.2]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [19.0 mg; LC (Gradient 1): retention time=6.20 minutes; Ms: (m+1) within the given range].

### EXAMPLE 14

### Preparation of ST2796 [AM9-SP02-Pro-Gly-NH₂, MW=433.2]

The peptidomimetic sequence indicated was synthesised on Rink Amide MBHA resin (66 mg,˜50 μmol) as described in Example 11. On completing the sequence and removing the Fmoc, the peptidomimetic, still on the resin, was treated by guanidinylation of the terminal amine function of β-alanine as described in Typical Procedure J. The cleavage product from the resin, purified by HPLC-RP and liophilised, ultimately yielded the expected molecule [10.0 mg; LC (Gradient 2): retention time=19.12 minutes; Ms: (m+1) within the given range].

### EXAMPLE 15

### Preparation of ST2863 [AM9-SP17-Pro-Gly-NH₂, MW=460.5]

The peptidomimetic sequence indicated was synthesised on Rink Amide MBHA resin (66 mg,˜50 μmol) as described in Example 13. The cleavage product from the resin, purified by HPLC-RP and liophilised, ultimately yielded the expected molecule [12.5 mg; LC (Gradient 2): retention time=18.12 minutes; Ms: (m+1) within the given range].

### EXAMPLE 16

### Preparation of ST2797 [PAM8-SP15-Pro-Gly-NH₂, MW=492.1]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [8.3 mg; LC (Gradient 2): retention time=29.03 minutes; Ms: (m+1) within the given range].

### EXAMPLE 17

### Preparation of ST2807 [PAM9-(SP31)₃-Pro-Gly-NH₂, MW=675.5]

The peptidomimetic sequence indicated was synthesised on Rink Amide MBHA resin (66 mg,˜50 μmol) as described in Example 11, repeating Typical Procedure C three times. The cleavage product from the resin, purified by HPLC-RP and liophilised, ultimately yielded the expected molecule [4.4 mg; LC (Gradient 2): retention time=22.54 minutes; Ms: (m+1) within the given range].

### EXAMPLE 18

### Preparation of ST2798 [PAM9-SP38-Pro-Gly-NH₂, MW=445.3]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [11.8 mg, LC (Gradient 2): retention time=18.83 minutes; Ms: (m+1) within the given range].

### EXAMPLE 19

### Preparation of ST2799 [Ac-arg-asp-val-leu-Beta1-NH₂, MW=722.3]

The peptidomimetic sequence indicated was synthesised on Rink Amide MBHA resin (66 mg,˜50 μmol) as described in Example 10. The cleavage product from the resin, purified by HPLC-RP and liophilised, ultimately yielded the expected molecule [11.9 mg; LC (Gradient 1): retention time=6.30 minutes; Ms: (m+1) within the given range].

### EXAMPLE 20

### Preparation of ST2801 [Ac-Arg-SP12-Beta2-Thr-NH₂, MW=629.3]

The peptidomimetic sequence indicated was synthesised on Rink Amide MBHA resin (66 mg,˜50 μmol) as described in Typical Procedures A, B, C and D (in this case collidine was used as the scavenger in the coupling reaction of the Fmoc-Arg(pbf)-Cl with the spacer 12 amine function and in Typical Procedures E and F). The cleavage product from the resin, purified by HPLC-RP and liophilised, ultimately yielded the expected molecule [2.9 mg; LC (Gradient 2): retention time=18.16 minutes; Ms: (m+1) within the given range].

### EXAMPLE 21

### Preparation of ST2804 [Ac-Arg-SP02-Beta2-Gly-NH₂, MW=601.0]

The peptidomimetic sequence indicated was synthesised on Rink Amide MBHA resin (66 mg,˜50 μmol) as described in Example 21. The cleavage product from the resin, purified by HPLC-RP and liophilised, ultimately yielded the expected molecule [2.7 mg; LC (Gradient 2): retention time=18.22 minutes; Ms: (m+1) within the given range].

### EXAMPLE 22

### Preparation of ST2806 [AM8-SP38-Beta6-NH₂, MW=571.3]

In a polypropylene reactor equipped with a cap and with a porous frit and drainage cock on the bottom, the Rink Amide MBHA resin (135 mg,˜100 μmol) was treated as described in Typical Procedure I. The synthesis of the peptidomimetic sequence was completed on the G8 constructed on resin (80 mg of resin˜50 μmol), as described in the Typical Procedures B, C and F. The material obtained from the cleavage from the resin, purified by HPLC-RP and liophilised, ultimately yielded the expected product [0.9 mg; LC (Gradient 1): retention time=6.78 minutes; Ms: (m+1) within the given range].

### EXAMPLE 23

### Preparation of ST2805 [NH₂-arg-SP02-Beta5, MW=578.3]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [0.3 mg, LC (Gradient 2): retention time=20.70 minutes; Ms: (m+1) within the given range].

### EXAMPLE 24

### Preparation of ST2825 [PAM4-SP19-Beta8-NH₂, MW=591.0]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [9.3 mg; LC (Gradient 1): retention time=12.28 minutes; Ms: (m+1) within the given range].

### EXAMPLE 25

### Preparation of ST2828 [H-SP32-Beta3-NH₂, MW=384.1]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [33.3 mg; LC (Gradient 1): retention time=7.62 minutes; Ms: (m+1) within the given range].

### EXAMPLE 26

### Preparation of ST3324 [PAM11-SP19-Beta8-NH₂, MW=572.7]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [9.4 mg; LC (Gradient 1): retention time=17.30 minutes; Ms: (m+1) within the given range].

### EXAMPLE 27

### Preparation of ST3374 [PAM10-SP6-Beta8-NH₂, MW=514.99]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [1.6 mg; LC (Gradient 1): retention time=10.15 minutes; Ms: (m+1) within the given range].

### EXAMPLE 28

### Preparation of ST3375 [PAM3-SP30-Beta8-NH₂, MW=557.07]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [0.6 mg; LC (Gradient 1): retention time=11.23 minutes; Ms: (m+1) within the given range].

### EXAMPLE 29

### Preparation of ST2793 [PAM8-SP20-Beta3-NH₂, MW=569.2]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [3.5 mg; LC (Gradient 1): retention time=10.02 minutes; Ms: (m+1) within the given range].

### EXAMPLE 30

### Preparation of ST2941 [PAM3-SP33-beta8-NH₂, MW=533.0]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [1.3 mg; LC (Gradient 1): retention time=10.08 minutes; Ms: (m+1) within the given range].

### EXAMPLE 31

### Preparation of ST2826 [PAM6-SP20-Beta8-NH₂, MW=565.2]

The peptidomimetic sequence indicated was synthesised on Rink Amide MBHA resin (66 mg,˜50 μmol) as described in Example 11. The cleavage product from the resin, purified by HPLC-RP and liophilised, ultimately yielded the expected molecule [5.5 mg; LC (Gradient 1): retention time=8.87 minutes; Ms: (m+1) within the given range].

### EXAMPLE 32

### Preparation of ST2926 [H-Arg-Gly-AzaVal-Val-Pro-Gly-NH₂, MW=583.7].

The peptidomimetic sequence indicated was synthesised on Rink Amide MBHA resin (66 mg,˜50 μmol) as described in Example 11, except the formation of azavaline that was performed according to the description in Typical Procedure H. The cleavage product from the resin, purified by HPLC-RP and liophilised, ultimately yielded the expected molecule [4.9 mg; LC (Gradient 1): retention time=4.98 minutes; Ms: (m+1) within the given range].

### EXAMPLE 33

### Preparation of ST3032 [Ac-Azagly-Azagly-pro-leu-val-asp-arg-NH₂, MW=755.8].

The peptidomimetic sequence indicated was synthesised on Rink Amide MBHA resin (66 mg,˜50 μmol) as described in Example 11, except the formation of azaglycine that was performed according to the description in Typical Procedure G. The cleavage product from the resin, purified by HPLC-RP and liophilised, ultimately yielded the expected molecule [10.0 mg; LC (Gradient 1): retention time=8.25 minutes; Ms: (m+1) within the given range].

### EXAMPLE 34

### Preparation of ST2927 [Ac-Arg-Asp-Azagly-Val-Pro-Gly-NH₂, MW=641.7].

The peptidomimetic sequence indicated was synthesised on Rink Amide MBHA resin (66 mg,˜50 μmol) as described in Example 11, except the formation of azaglycine that was performed according to the description in Typical Procedure G. The cleavage product from the resin, purified by HPLC-RP and liophilised, ultimately yielded the expected molecule [18.2 mg; LC (Gradient 1): retention time=5.17 minutes; Ms: (m+1) within the given range].

### EXAMPLE 35

### Preparation of ST2930 [Ac-thr-Azagly-pro-leu-val-asp-arg-NH₂, MW=798.9].

The peptidomimetic sequence indicated was synthesised on Rink Amide MBHA resin (66 mg,˜50 μmol) as described in Example 11, except the formation of azaglycine that was performed according to the description in Typical Procedure G. The cleavage product from the resin, purified by HPLC-RP and liophilised, ultimately yielded the expected molecule [19.1 mg; LC (Gradient 1): retention time=6.20 minutes; Ms: (m+1) within the given range].

### EXAMPLE 36

### Preparation of ST2920 [Ac-Arg-Asp-Val-AzaVal-Pro-Gly-NH₂, MW=681.8].

The peptidomimetic sequence indicated was synthesised on Rink Amide MBHA resin (66 mg,˜50 μmol) as described in Example 11, except the formation of azavaline that was performed according to the description in Typical Procedure H. The cleavage product from the resin, purified by HPLC-RP and liophilised, ultimately yielded the expected molecule [11.7 mg; LC (Gradient 1): retention time=5.88 minutes; Ms: (m+1) within the given range].

### EXAMPLE 37

### Preparation of ST2928 [Ac-Arg-Asp-AzaLeu-Val-Pro-Gly-NH₂ MW=697.8].

The peptidomimetic sequence indicated was synthesised on Rink Amide MBHA resin (66 mg,˜50 μmol) as described in Example 11, except the formation of azaleucine that was performed according to the description in Typical Procedure H. The cleavage product from the resin, purified by HPLC-RP and liophilised, ultimately yielded the expected molecule [12.00 mg; LC (Gradient 1): retention time=5.18 minutes; Ms: (m+1) within the given range].

The compounds according to the present invention are useful as medicaments and as means for use in biological assays.

Their activity consists in the inhibition of the homodimerisation of the protein MyD88, thus proving capable of inhibiting a greater number of pro-inflammatory signals and constituting a more effective therapeutic agent.

In its general application, the present invention provides for the use of formula (I) compounds for the preparation of a medicament useful for the treatment of diseases deriving from dysregulation of the signalling system of the TLR/IL-R1 receptor system.

Knowledge of the field enables experts to determine, on the basis of the molecular biology mechanism indicated above, the diseases that can be treated.

The diseases that can be treated according to the present invention are selected from the group consisting of inflammatory and auto-immune diseases; cardiovascular and atherogenic diseases; sepsis and shock; and transplant rejection.

Examples of inflammatory and autoimmune diseases are arthritis, chronic inflammatory bowel disease (IBD), psoriasis, type 1 diabetes, multiple sclerosis, asthma, and systemic lupus erythematosus.

Examples of cardiovascular and atherogenic diseases are myocardial infarct, viral myocarditis, atherosclerosis, vein graft atherosclerosis, thrombosis, re-stenosis, re-stenosis due to stents and re-stenosis due to angioplasty.

Examples of non inflammatory diseases include cancer and AIDS

The medicaments according to the present invention will contain an efficacious amount of formula (I) compounds, determined according to normal clinical trials. The primary care physician will then determine the posology according to the type of disease to be treated, the patient's condition and any concomitant therapies.

Biological Assays

The compounds which are the subject of the present invention were subjected to tests of a biological nature in order to identify their ability to inhibit the homodimerisation of MyD88 partly or totally and thus to modulate the activation of NF-kB. To this end, three types of assays were used: a) the double hybrid assay in yeast, a brief description of which is provided here below, b) the NF-kB inhibition assay, also reported here below and c) the reporter gene assay of luciferase activity, also reported here below. Compounds considered as being active are those found to be active in either of the three biological assays.

a) Double Hybrid Assay

The double hybrid system in the yeast Saccharomyces cerevisiae is based on the ability to reconstitute in vitro the transcription factor GAL4, which can be divided into two functional domains, the activation domain (AD) and the binding domain (BD) (Field, S.; Song, O.; Nature, 1989, 340:245-247; Chien, C. T.; et al.; Proc. Nat. Acad. Sci. USA; 1991, 88:9578-9582). If, by means of molecular biology techniques, these two domains are fused with two proteins capable of interacting, the result will be the functional reconstitution of GAL4, which will activate the transcription of a number of reporter genes under the control of its own upstream activation sequence (UAS). The transcription of the reporter genes under the control of the UAS of GAL4 will permit the synthesis of enzymes which are important for growth in selective medium. The assay is conducted using 384-well plates on the bottom of which is present a silicone matrix in which a fluorescent substance is incorporated, the emission of which is sensitive to levels of oxygen (Wodnicka, M.; et al.; J. Biomol. Screen; 1995, 5:141-152). When the interaction occurs between the two proteins fused with the BD and AD domains of GAL4, respectively, in the double hybrid system, the yeast will be capable of growing in the selective medium and will consume oxygen, and the fluorescence emitted, which will increase proportionately in the course of time, will be detectable with a suitable fluorescence reader (Fusion, Perkin Elmer). If the yeast is placed in the presence of molecules capable of inhibiting this interaction, the transcription of the reporter genes will be reduced and thus the ability to grow in minimal medium will be slowed down with a reduction of the fluorescence signal.

The vectors (pGBKT7 and pGADT7) used for the expression of MyD88 fused with the domains AD and BD were supplied by Clontech, as was the yeast strain AH109 (MATa, trp1-901 leu2-3 112 ura3-52 his3-200 gal4Δ gal80Δ used for the co.transformation and the minimal media SGd/-Leu/-Trp e SGd/-Ade/-His/-Leu/-Trp. The 384-well plates used for the assay were supplied by BD Biosciences (Oxygen BioSensor Plates) and the instrument for the measurement of the fluorescence is the Perkin Elmer Fusion device. The strain AH109 co-transformed with the two gene fusions (AD-MyD88 and BD-MyD88) is preinoculated in 2 mL of SGd/-Leu/-Trp and incubated overnight at 30° C. under stirring at 200 rpm; the preinoculum is then diluted (1/20) in 100 mL of SGd/-Ade/-His/-Leu/-Trp for each well of the 384-well plate in the presence of the molecule to be assayed (final concentration 100 mM). The plate is incubated in the Fusion at 30° C. and the fluorescence emitted by each individual well is measured using an excitation wavelength of the fluorophore of 485 nm and reading the emission at 630 nm from the bottom of the plate every 90 minutes for a total of 25 readings. Fluorescence intensity is an arbitrary unit, and therefore normalisation is necessary: the fluorescence intensity of a well at time n must be divided by the initial fluorescence intensity for the same well.

Analysis of Fluorescence Increase Curves

The fluorescence increase curves generated by the double hybrid assay were analysed by means of a system specifically elaborated for this type of investigation (software elaborated by Chrysallis s.a.s. software-house, granted under unlimited usage license to Tecnogen S.C.p.A.).

The curve analysis system is capable of interfacing directly with the files produced by the data acquisition instruments without any need for pretreatment of the data, thus immediately allowing a qualitative-type analysis.

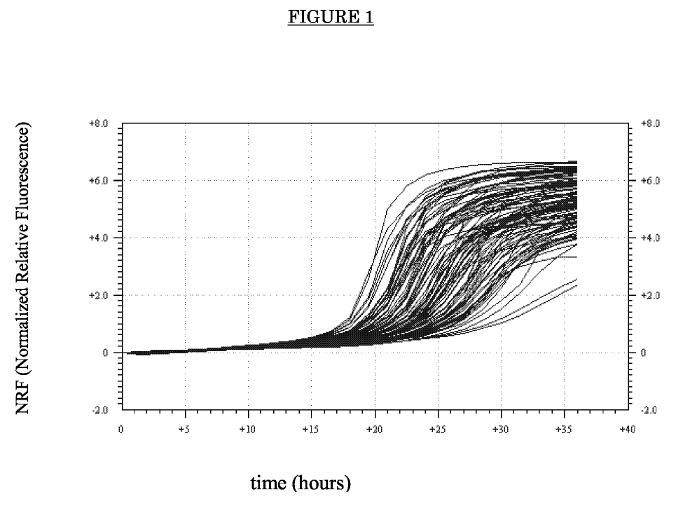

By way of an example, FIG. 1/4 presents the curves obtained from one plate that contained most of the class 2 compounds. The curves for the increase in fluorescence are represented as NRF (normalised relative fluorescence in relation to time 0) as a function of time.

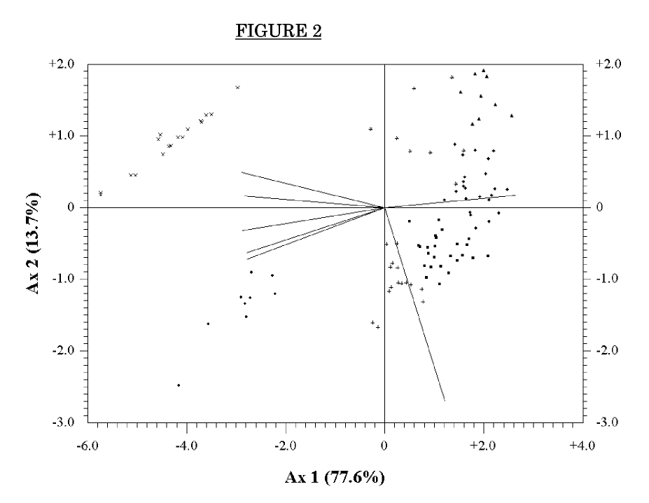

The software provides quantitative parameters capable of describing the characteristics of the individual curves, which, as shown in the figure, present distinctly different aspects. In particular, seven different descriptors were used: slope (slope of the plateau), level (height of the plateau), range (plateau range), ΔT (growth time), t½ (time to grow to 50% of plateau height), factor (“hump” factor), and Tau (sigmoidal τ). By means of these quantitative parameters, it is possible to obtain a multidimensional representation of the set of experimental curves. Starting from this, and using extraction techniques for the main components, a two-dimensional representation of the data is constructed such as to highlight as clearly as possible the differences between the curves. The factorial plane that follows “explains” the variability identified between the curves (represented by different symbols) obtained from the plate analysed, attributing them to the various parameters or to combinations thereof (FIG. 2/4).

The variability explained by the first two factorial axes is 77.6%+13.7%=91.3% so that the two-dimensional graph on the first factorial plane is more than sufficient to display to a good degree of approximation the spatial dispersion of the curves. The first factorial axis tends to separate the curves that have a high plateau level, which we find to the right of the axis, from those that have a lower plateau level, which, on the contrary, will be to the left of the axis. The latter also present the characteristic of having higher ΔT, Tau, t½, range and slope than the curves lying to the right. The values of these descriptors are higher, the further they lie from the intersection of the two axes.

This type of representation allows the identification of “groups” of curves with similar behaviours (curves with the same symbols correspond to curves with similar characteristics) and to identify clearly which curves have been generated by compounds that have influenced the consumption of oxygen and thus the growth of the yeast.

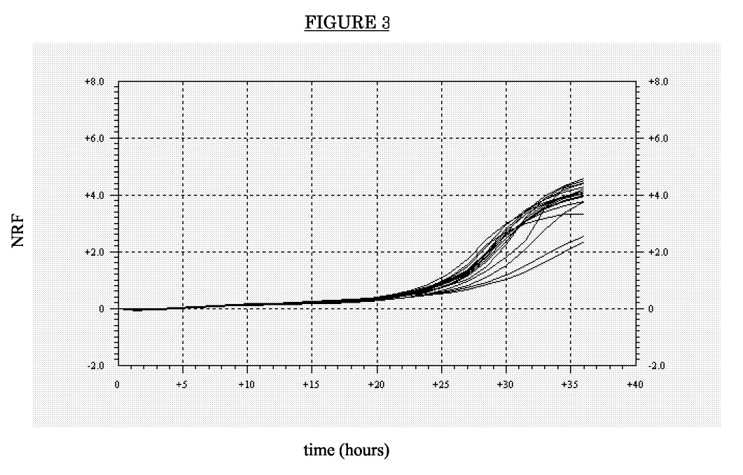

Selected therefore from this plate, which provided this representation (FIG. 3/4), were the group of curves indicated in FIG. 2/4 by the symbol x, which present a lower plateau level and higher growth time (ΔT) and tau values compared to the mean value of the curves generated by the compounds present on this plate, as reported in the statistics here below:

ΔT=36.001±0.0 (14.615±10.252)

Level=2.884±0.667 (4.763±1.131)

Slope=0.263±0.077 (0.125±0.096)

Range=0.801±0.279 (0.364±0.294)

Tau=30.005±0.0 (8.805±12.146)

t ½=33.006±0.012 (25.142±4.738)

Factor=0.006±0.002 (0.402±0.258)

The values indicated in brackets are the mean values obtained for that parameter in the plate analysed.

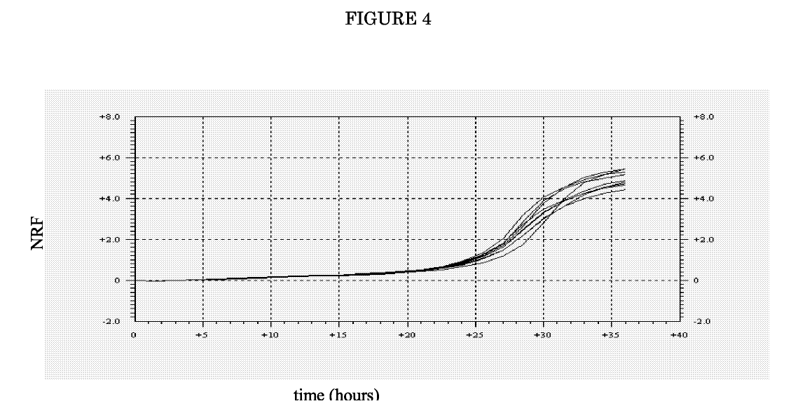

In addition, the group of curves indicated in FIG. 2/4 by the symbol ● were selected from this plate and, despite the fact that they present a factor greater than the mean and a ΔT value below the mean, they present t ½, ranges and slopes greater than the mean of the curves generated by the compounds present on the plate, as reported in the statistics here below (FIG. 4/4):

ΔT=22.687±1.577 (14.615±10.252)

Level=4.044±0.377 (4.763±1.131)

Slope=0.261±0.071 (0.125±0.096)

Range=0.746±0.201 (0.364±0.294)

Tau=30.005±0.003 (8.805±12.146)

t ½=33.004±0.0 (25.142±4.738)

Factor=0.619±0.056 (0.402±0.258)

The analysis system described was applied in the same way to all the plates used for the selection of the compounds according to the present invention and yielded the following results:

| NAME | SEQUENCE |
|---|---|
| CLASS 1 | |
| ST2402 | Ac-arg-asp-val-leu-pro-gly-NH₂ |
| ST2565 | Ac-thr-gly-pro-leu-val-asp-arg-NH₂ |
| ST2842 | 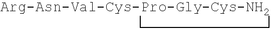 |
| ST2946 | Ac-arg-asn-val-leu-pro-gly-NH₂ |
| ST2947 | Ac-arg-asp-val-val-pro-gly-NH₂ |
| CLASS 2 | |
| ST2793 | PAM8-SP20-Beta3-NH₂ |
| ST2806 | AM8-SP38-Beta6 |
| ST2825 | PAM4-SP19-Beta8-NH₂ |
| ST2826 | PAM6-SP20-Beta8-NH₂ |
| ST2827 | PAM6-(SP39)_{2-Beta3-NH}₂ |
| ST2828 | SP32-Beta3-NH₂ |
| ST2848 | AM8-SP12-Beta7 |
| ST2849 | PAM8-SP33-Beta4-NH₂ |
| ST2851 | PAM3-SP39-Beta3-NH₂ |
| ST2852 | PAM6-(SP39)₄-BETA3-NH₂ |
| ST2935 | PAM8-SP12-Beta3-NH₂ |
| ST2936 | PAM10-SP19-Beta3-NH₂ |
| ST2937 | AM8-SP33-Beta5 |
| ST2938 | PAM3-SP39-Beta4-NH₂ |
| ST2940 | AM4-SP33-Beta3-NH₂ |
| ST2941 | PAM3-SP33-Beta8-NH₂ |
| ST3374 | PAM10-SP6-Beta8-NH₂ |
| CLASS 3 | |
| ST2791 | PAM8-SP2-Beta1-Thr-NH₂ |
| ST2795 | PAM6-SP18-Pro-Gly-NH₂ |
| ST2796 | AM9-SP2-Pro-Gly-NH₂ |
| ST2797 | PAM8-SP15-Pro-Gly-NH₂ |
| ST2853 | AM1-GLY-SP30-Pro-Gly-NH₂ |
| ST2854 | Beta7-SP2-arg-NH₂ |
| ST2855 | Ac-ARG-SP12-Beta1-THR-NH₂ |
| ST2856 | PAM8-ASP-VAL-VAL-Pro-Gly-Gly-NH₂ |
| ST2857 | PAM10-SP18-Pro-Gly-NH₂ |
| ST2858 | PAM3-SP18-Pro-Gly-NH₂ |
| ST2859 | PAM6-SP12-Pro-Gly-NH₂ |
| ST2862 | AM9-SP15-Pro-Gly-NH₂ |
| ST2863 | AM9-SP17-Pro-Gly-NH₂ |
| ST2864 | Ac-Gly-PRO7-SP30-Arg-NH₂ |
| ST2867 | Beta6-val-val-asp-arg-NH₂ |
| ST2868 | Ac-Gly-Pro-SP2-Arg-NH₂ |
| ST2869 | Ac-Pro-Gly-SP2-ARG-NH₂ |
| ST2870 | PAM8-(SP31)₄-Pro-Gly-NH₂ |
| ST2942 | Beta5-SP38-His-OH |
| ST2943 | PAM10-SP2-Pro-Gly-NH₂ |
| ST2944 | PAM6-SP14-Pro-Gly-NH₂ |
| ST2945 | PAM9-(SP17)₂-Pro-Gly-NH₂ |

B) NF-kB Inhibition Assay

The activation of NF-kB is an event that takes place downstream of the homodimerisation of MyD88 and its binding to the intracytoplasmic portions of number of receptor complexes. The ability of the compounds which are the subject of the present invention to inhibit the activation of NF-kB downstream of the amplification cascade of the signal triggered by IL1α was therefore evaluated.

All the molecules were assayed beforehand with the MTT cell viability test, verifying that the dose of compound used in the NF-kB inhibition assay was lower than the toxic dose.

HeLa* cells were cultured in EMEM (EBSS) medium supplemented with 2 mM glutamine+1% non-essential amino acids+7.5% FBS (Foetal Bovine Serum)+10 ml/l of a solution of penicillin-streptomycin (10,000 units/ml penicillin and 10 mg/ml streptomycin).

(All cell media and the various components were purchased from Sigma-Aldrich. *HeLa cell line Human Negroid cervix epithelioid carcinoma Human from SIGMA ALDRICH ECACC Ref No: 93021013. IL1α*=SIGMA I2778 Interleukin-1-alpha IL1a Human, recombinant expressed in E. coli).

The cells were used after a number of cell passages ranging from 14 to 35.

Cells are seeded in 6-well plates at a density of 300,000 cells/well in complete medium and incubated overnight at 37° C., 5% CO₂.

After approximately 18 hours, the complete medium is eliminated, cells are washed twice with PBS 1× and 1 ml of FBS-free medium is added to each well.

The molecules to be tested are subsequently added to the medium at a concentration of 100 μM. Cells are then incubated for 6 hours at 37° C., 5% CO₂.

All the molecules assayed were dissolved in DMSO.

An equivalent volume of DMSO is added to the negative controls, i.e. cells not treated with the molecules.

At the end of the treatment with the molecules, the cells are stimulated with 5 ng/ml of IL1α* for 30 minutes and incubated at 37° C., 5% CO₂.

An equivalent volume of PBS/BSA 0.4% (solution used to dissolve IL1α) is added to the negative controls, i.e. non-stimulated cells.

After stimulation with IL1α the cells are washed twice with PBS 1× and collected by cell scraping.

The cells are then centrifuged at 800 rpm, 4° C., 10 min. The supernatants are eliminated. The pellets are re-suspended in lysis buffer**, incubated for 10 minutes at 4° C. and centrifuged at maximum speed at 4° C. The pellets are eliminated and the supernatants frozen at −80° C.

The total protein content is subsequently measured by Bradford Assay using, as the standard, BSA at known concentration, and as used in the ELISA assay as described here below.

Activation/inhibition of NF-kB is evaluated using the Trans AM# (Active Motif) kit.

Il Trans AM kit permits detection of the activation of NF-kB induced by IL1α, through a calorimetric reaction that is obtained at the end of a routine ELISA assay.

The kit provides 96-well plates derivatised with an oligonucleotide that contains the consensus site of NF-kB (5′-GGGACTTT-CC-3′). This oligonucleotide specifically binds only the active form of NF-kB which is released after stimulation with IL1α. The first antibody supplied for detecting NF-kB recognises the p65 epitope which is accessible only when the transcriptional factor is active and binds its target DBNA sequence.

The secondary antibody supplied is conjugated to horse radish peroxidase which, with the addition of a chromogenic substrate, makes it possible to obtain a calorimetric reaction that can be evaluated spectrophotometrically at the wavelength of 450 nm.

The samples are assayed in duplicate and 10 μg of each extract are loaded into each well.

At the end of the assay, the % inhibition is calculated by processing the values obtained in the spectrophotometric readings as follows:

IL*-C*/IL-C=% activation of NF-kB.

IL*=A₄₅₀ of cells stimulated and treated with the study molecules.

C*=A₄₅₀ of cells not stimulated and treated with the study molecules.

IL=A₄₅₀ of cells stimulated with IL1α and not treated with the molecules.

C=A₄₅₀ of cells not stimulated and not treated.

The greater the NFkB activation, the lower will be the activity of the molecule assayed.

All the reagents used to perform the cell lysis and the ELISA assay are supplied by the kit used.

Compounds considered positive in the NF-kB inhibition assay were those that gave a percentage inhibition ≧15%.

| | | |
|---|---|---|
| | | % NF-κB |
| | | INHIBI- |
| NAME | SEQUENCE | TION |

CLASS 1

| | | |
|---|---|---|
| ST2565 | Ac-thr-gly-pro-leu-val-asp-arg-NH₂ | 30 |

| | | % NF-kB |
|---|---|---|
| | | INHIBI- |
| NAME | SEQUENCE | TION |
| CLASS 2 | | |
| ST3375 | PAM3-SP30-Beta8-NH₂ | 33 |
| ST2828 | SP32-Beta3-NH₂ | 27 |
| ST2825 | PAM4-SP19-Beta8-NH₂ | 26 |
| ST2806 | AM8-SP38-Beta6 | 24 |
| ST2826 | PAM6-SP20-Beta8-NH₂ | 23 |
| ST2793 | PAM8-SP20-Beta3-NH₂ | 18 |
| ST2863 | AM9-SP17-Pro-Gly-NH₂ | 17 |
| ST2941 | PAM3-SP33-BETA8-NH2 | 17 |
| | | % NF-kB |
| | | INHIBI- |
| NAME | SEQUENCE | TION |
| CLASS 3 | | |
| ST2804 | Ac-Arg-SP2-Beta2-Gly-NH₂ | 34 |
| ST2807 | PAM9-(SP31)₃-Pro-Gly-NH₂ | 33 |
| ST2794 | PAM9-Asp-Val-Val-Beta2-NH₂ | 25 |
| ST2799 | Ac-arg-asp-val-leu-Beta1-NH₂ | 23 |
| ST2792 | PAM9-SP2-Beta2-Thr-NH₂ | 22 |
| ST2797 | PAM8-SP15-Pro-Gly-NH₂ | 20 |
| ST2798 | PAM9-SP38-Pro-Gly-NH₂ | 18 |
| ST2796 | AM9-SP2-Pro-Gly-NH₂ | 17 |
| ST2801 | Ac-Arg-SP12-Beta2-Thr-NH₂ | 17 |
| ST2864 | Ac-Gly-Pro-SP30-Arg-NH2 | 16 |
| ST2805 | NH₂-arg-SP2-Beta5 | 15 |

c) Reporter Gene Assay of Luciferase Activity in Human Intestinal CaCo2 Epithelial Cells Stimulated with IL-1.

It is based on transient co-transfection of CaCo2 human intestinal epithelial cells with a reporter gene plasmid whose luciferase reporter gene expression is under control of the IL-1-responsive human IL-8 promoter gene region. The second plasmid in co-transfection is a vector coding for a control Renilla luciferase reporter gene, whose constitutive expression is used to estimate unspecific cellular toxicity of tested compounds. The read-out of the assay has been defined as the Relative Response Ratio (RRR) for the firefly and the Renilla luciferase, as follows:

$$RRR = \frac{( experimental sample ) - ( negative control )}{( positive control ) - ( negative control )} ⨯ 100$$

where the experimental sample is the value in cont per second (cps) of the experimental reporter luminescence, as defined for any unknown sample. The positive control is the value in cps of the reporter luminescence, as defined for the sample which identifies the maximal induction by IL-1 in the absence of reference inhibitor compound. The negative control is the value in cps of the reporter luminescence, as defined for the sample which identifies the absence of induction by IL-1. For the non-inducible Renilla luciferase, the RRR has been defined as follows:

$$RRR = \frac{( experimental sample )}{( positive control )} ⨯ 100.$$

The RGA is the read-out of the above experiments, and it was run as follows:

Plate 6×10⁶ adherent CaCo2 cells on 10 cm plates. After 18-24 hours culture medium is changed, using 9 mL of D-MEM+glutamine 580 mg/L, without FBS and antibiotics. For each plate, 26 μg of IL-1-responsive reporter vector DNA (pGL2-NA-INT) and 4 μg of control reporter vector DNA are then dissolved in 500 μL Optimem (Invitrogen). Lipofectamine 2000 Reagent (Invitrogen) 30 μL are then added to 500 μL Optimem; this latter reaction mix is incubated for 5 min at room temperature. The DNA mix is then added drop by drop to the Lipofectamine 2000 mix. The resulting DNA/Lipofectamine mix is incubated for 20 min at room temperature and it is added drop by drop over the culture plate, by gently rocking the plate itself. Culture plates are then incubated for about 6 hours at 37° C. and 5% CO₂. Cells are then trypsinized and transferred in 96-well plates, at 5×10⁴ cells/well in 100 μL of culture medium (D-MEM with FBS 1%,+glutamine 580 mg/L), as required by the experimental design. Afterwards, cells were incubated for 16-18 hours at 37° C. and 5% CO₂ and treated as follows:
Take out culture medium from each well.
Add 60 μL of fresh culture medium to control cell wells.
Add 40 μL of fresh culture medium to IL-1-treated wells.
Add 40 μL of fresh culture medium and 20 μL of substance at a concentration of 100 μM and 0.4% DMSO to inhibitor substance wells.
Add 20 μL fresh culture medium supplemented with 0.4% DMSO in IL-1-treated wells and in control cell wells.
Incubate at 37° C. and 5% CO₂ for 4 hours.
After 2 hours, stimulate with 20 μL of IL-1 (500 pg/mL) for two additional hours at 37° C. and 5% CO₂.
Appropriate amount of luciferase standard protein is included in additional assay wells.
Add for each well 80 μL of firefly luciferase substrate (Dual-Glo Luciferase Assay System reagent).
Incubate at room temperature for 10 min.
Read the firefly luciferase output on the Veritas lumenometer (Turner BioSystems).
Immediately add to each well 80 μL of the Renilla luciferase substrate (Dual-Glo Luciferase Assay System reagent).
Incubate at room temperature for 10 min.
Read the Renilla luciferase output on the Veritas lumenometer (Turner BioSystems).

Compounds considered positive in the RGA assay were those that gave a percentage inhibition ≧20%.

| | | |
|---|---|---|
| NAME | SEQUENCE | % INHIBITION |

CLASS 2

| | | |
|---|---|---|
| ST2828 | SP32-Beta3-NH₂ | 24 |
| ST2825 | PAM4-SPT9-Beta8-NH₂ | 71 |
| ST2793 | PAM8-SP20-Beta3-NH₂ | 20 |
| ST3324 | PAM11-SP19-Beta8-NH₂ | 51 (80 μM) |
| CLASS 3 | | |
| ST2926 | H-Arg-Gly-AzaVal-Val-Pro-Gly-NH₂ | 20 |
| ST3032 | Ac-Azagly-Azagly-pro-leu-val-asp-arg-NH₂ | 21 |
| ST2927 | Ac-Arg-Asp-Azagly-Val-Pro-Gly-NH₂ | 22 |
| ST2930 | Ac-thr-Azagly-pro-leu-val-asp-arg-NH₂ | 24 |
| ST2920 | Ac-Arg-Asp-Val-AzaVal-Pro-Gly-NH₂ | 25 |
| ST2928 | Ac-Arg-Asp-AzaLeu-Val-Pro-Gly-NH₂ | 29 |
| ST2797 | PAM8-SP15-Pro-Gly-NH₂ | 31 |

In accordance with the present invention, the pharmaceutical compositions contain at least one active ingredient in an amount such as to produce a significant therapeutic effect. The compositions covered by the present invention are entirely conventional and are obtained with methods that are common practice in the pharmaceutical industry, such as, for example, those illustrated in Remington's Pharmaceutical Science Handbook, Mack Pub. N.Y.—latest edition. According to the administration route opted for, the compositions will be in solid or liquid form, suitable for oral, parenteral or intravenous administration. The compositions according to the present invention contain, along with the active ingredient, at least one pharmaceutically acceptable vehicle or excipient. Formulation adjuvants may be particularly useful, e.g. solubilising agents, dispersing agents, suspension agents or emulsifying agents.

In view of the peptidic nature of the compounds according to the present invention, technicians with average experience in this field will be able to determine the advisability of formulating the compounds in pharmaceutical compositions for oral administration in gastroprotected or controlled-release form.

1. Peptidic and/or peptidomimetic compounds with formula (I)
(X⁻)AA₁-AA₂-AA₃-AA₄-AA₅-AA₆-AA₇ (I)
in which:
X⁻ is an anion of a pharmacologically acceptable acid, or is absent;
each of the groups AA₁-AA₇, which may be the same or different, is an amino acid or amino acid mimetic with the following meanings:
AA₁=is the residue of L-arginine (Arg), D-arginine (arg), L-histidine (His), D-histidine (his), or an arginino-mimetic group, where what is meant by arginino-mimetic is a chemical structure that substitutes for arginine and modulates the basicity of the functional group, from the basicity of arginine to zero basicity, with formulas (II), (III) and (IV) 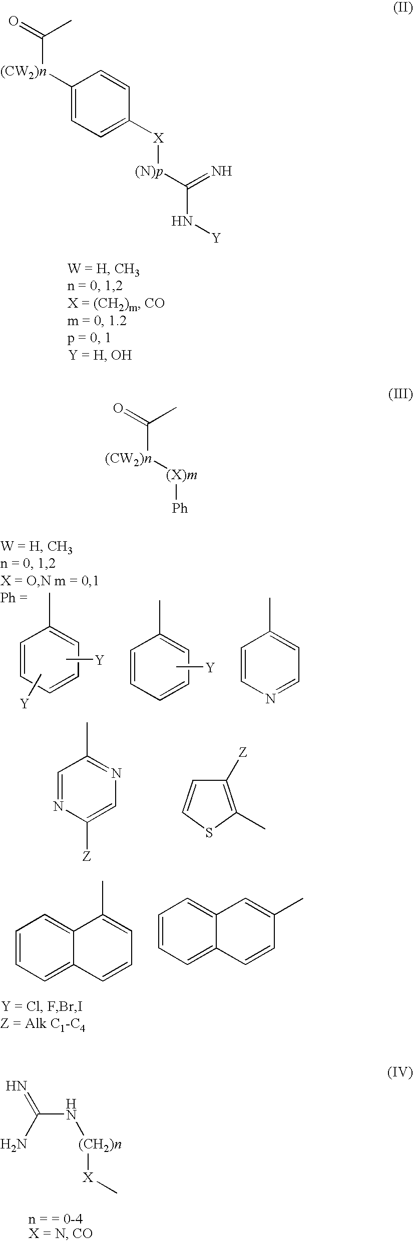
or is absent;
AA₂=L-aspartic acid (Asp), D-aspartic acid (asp), L-asparagine (Asn), D-asparagine (asn), glycine (gly or Gly), or is absent;
AA₃=L-valine (Val), D-valine (val), azavaline (Aza-Val), azaglycine (Azagly), azaleucine (AzaLeu);
AA₄=L-leucine (Leu), D-leucine (leu), L-valine (Val), D-valine (val), L-cysteine (Cys), D-cysteine (cys), azaleucine (Aza-Leu), azavaline (Aza-Val), azaglycine (Azagly);
AA₂-AA₃-AA₄ together can be substituted by a spacer where what is meant by spacer is a hydrophobic chemical structure with a limited number of rotational freedom degrees that contains an aromatic linker ring variously substituted and functionalised, only one carboxylic acid group and only one primary amine group, engaged in amide bonds, with formula (V): 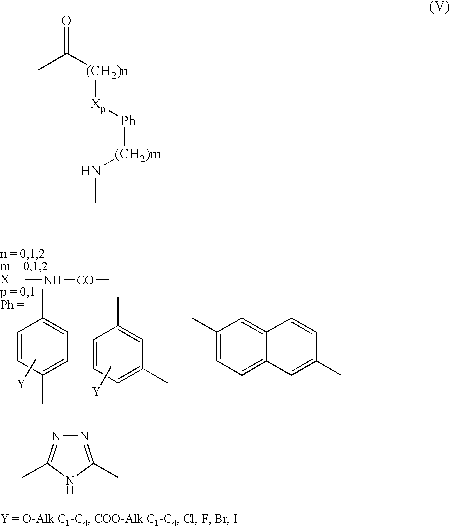
AA₅=L-proline (Pro), D-proline (pro), cis-4,5-(methano)-L-proline (cMe-Pro), cis-4,5-(methano)-D-proline (cMe-pro), trans-4,5-(methano)-L-proline (tMe-Pro), trans-4,5-(methano)-D-proline (tMe-pro);
AA₆=glycine (gly or Gly), sarcosine (Sar), azaglycine (Azagly);
AA₅-AA₆ together can be substituted by a β-turn mimetic, where what is meant by β-turn mimetic is a chemical structure which, by mimicking the central portion of the Pro-Gly β-turn, allows the molecule to take on a conformation useful for the formation of bonds with the protein MyD88, with formulas (VI) and (VII) 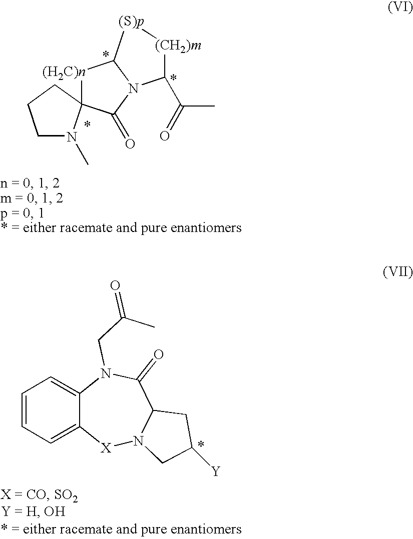
AA₇=is the residue of glycine (gly or Gly), azaglycine (Azagly), L-threonine (Thr), D-threonine (thr), L-cysteine (Cys), D-cysteine (cys), or is absent;
when AA₄=AA₇=Cys or cys there is a disulphide bridge between the two cysteines;
when some or all of AA₁, AA₂, AA₃, AA₄, AA₅, AA₆ and AA₇ are amino acids, these can be L or D and the sequence can be reversed or not;
the bond between the AA₁-AA₇ residues is always of the amide type;
the terminal amine group can be free or acylated with a pharmacologically acceptable radical useful for transporting the molecule, e.g., acetyl, formyl, benzoyl, propionyl, cyclohexyl, myristoyl; the terminal carboxyl can be in the form of carboxylic acid or primary amide.
the individual enantiomers, diastereoisomers, mixtures thereof and their pharmaceutically acceptable salts;
upon the following conditions:
that at least one of AA₁-AA₇ is not a natural amino acid among those indicated above, or
if all of AA₁-AA₇ are natural amino acids among those indicated above, said AA₁-AA₇ sequence is reversed.

2. Compounds according to claim 1, in which said arginino-mimetic is a chemical structure that substitutes for arginine and modulates the basicity of the functional group from the basicity of arginine to zero basicity.

3. Compounds according to claim 1, in which said spacer is a hydrophobic chemical structure with a limited number of rotational freedom degrees that contains an aromatic linker ring variously substituted and functionalised, only one carboxylic acid group and only one primary amine group engaged in amide bonds.

4. Compounds according to claim 1, in which said β-turn mimetic is a chemical structure that allows the molecule to take on a conformation useful for the formation of bonds with the protein MyD88.

5. Compounds according to claim 1, in which, in AA₁, the argininomimetic is selected from the group consisting of 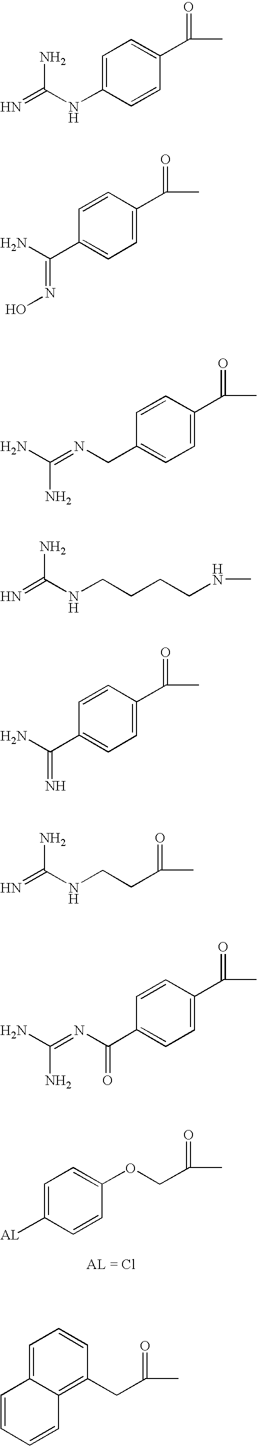 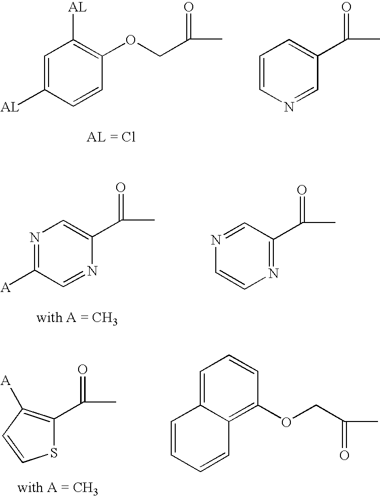
in which A is a straight or branched C₁-C₄ alkyl group; Al is a halogen atom selected from F, Cl, Br and I.

6. Compounds according to any one of claims 1, in which, when AA₂-AA₃-AA₄ are substituted with a spacer (SPX)ₙ where n=0-3 and is selected from the group consisting of 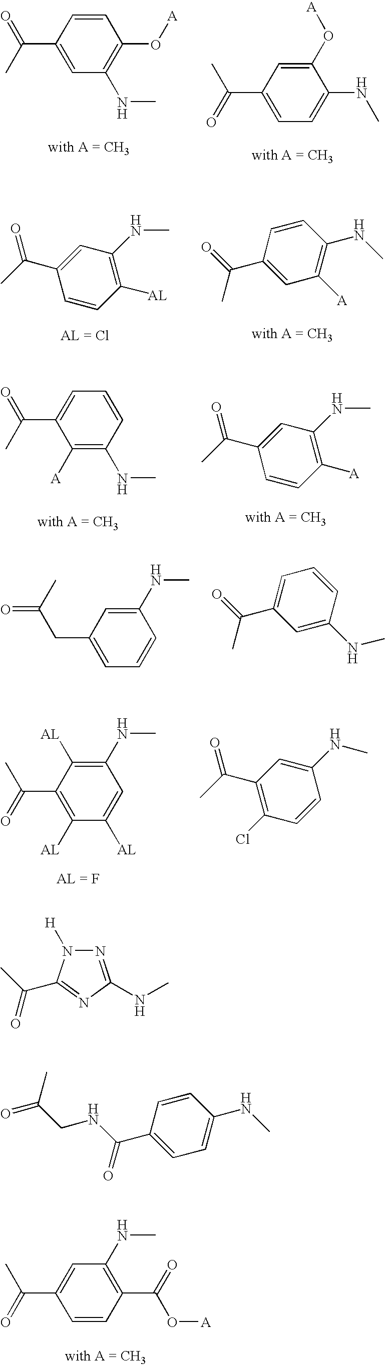 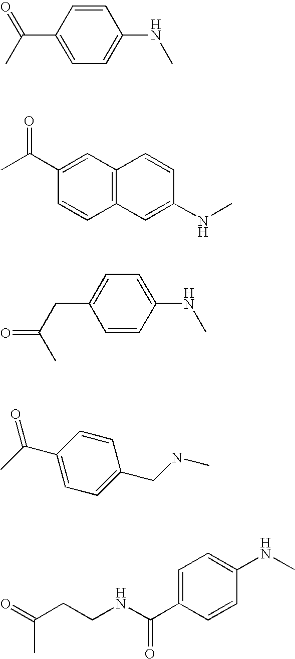
in which A is a straight or branched C₁-C₄ alkyl group; AL is a halogen atom selected from F, Cl, Br and I.

7. Compound according to any one of claims 1, in which said β-turn is selected from the group consisting of

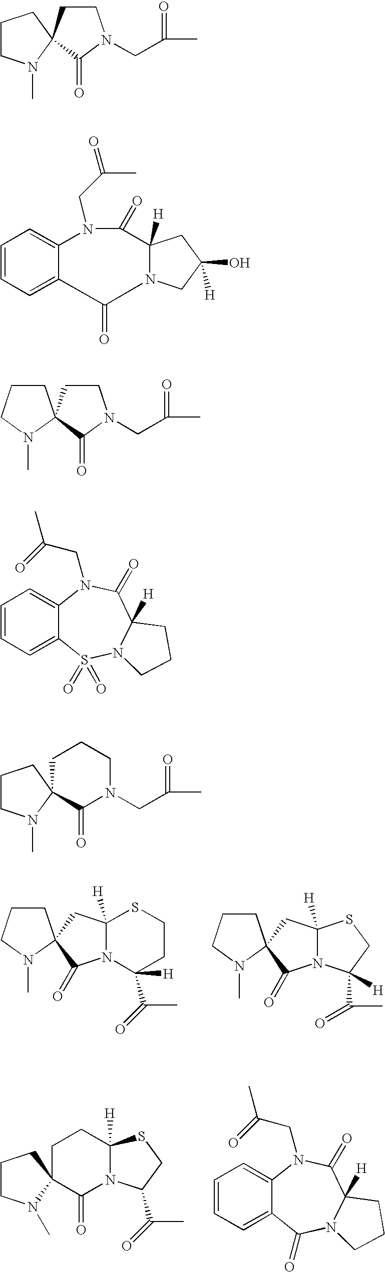

8. Compound according to claim 1, with the formula Ac-thr-gly-pro-leu-val-asp-arg-NH₂.

9. Compound according to claim 1, selected from the group consisting of:

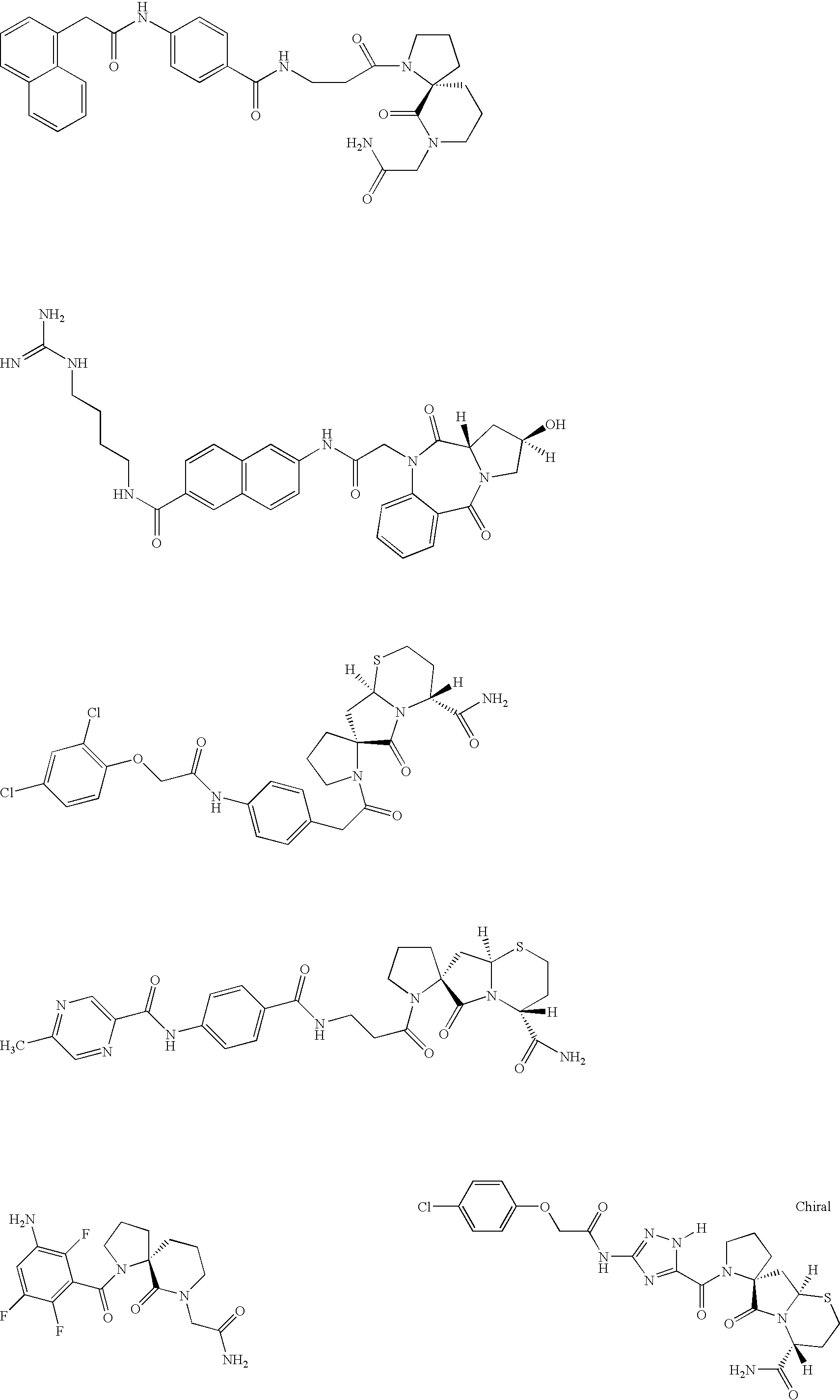

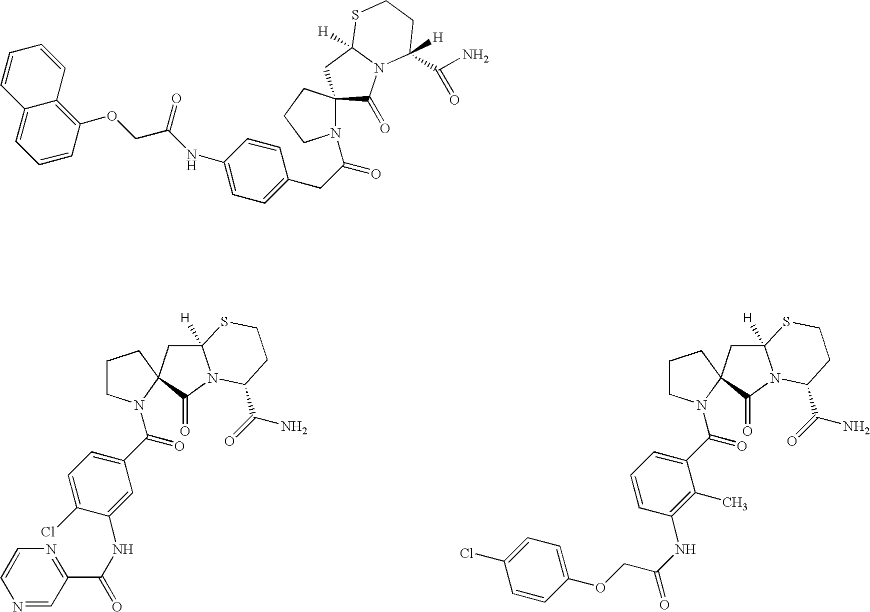

10. Compounds according to claim 1, in which AA₅-AA₆ are substituted with a β-turn mimetic.

11. Compound according to claim 10 with the formula

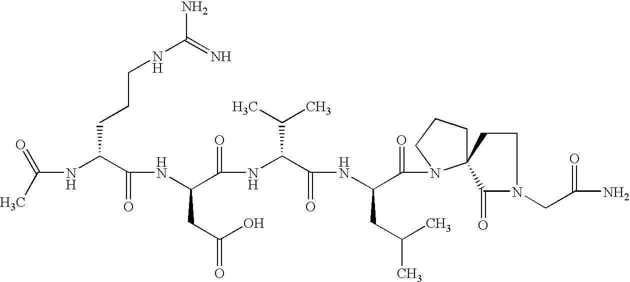

12. Compounds according to claim 1, in which AA₂-AA₃-AA₄ are substituted with a spacer and AA₅-AA₆ are substituted with a β-turn mimetic.

13. Compound according to claim 12, selected from the group consisting of:

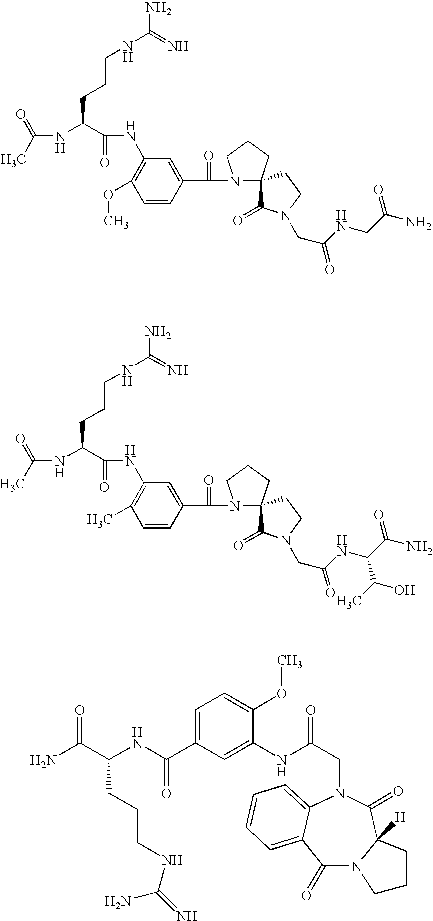

14. Compounds according to claim 1, in which AA1 is an argininomimetic and AA5-AA6 are substituted with β-turn mimetic.

15. Compound according to claim 14 with the formula

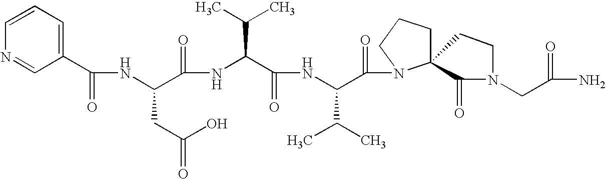

16. Compounds according to claim 1, in which AA₁ is an arginino-mimetic and AA₂-AA₃-AA₄ are substituted with a spacer.

17. Compounds according to claim 16, selected from the group consisting of:

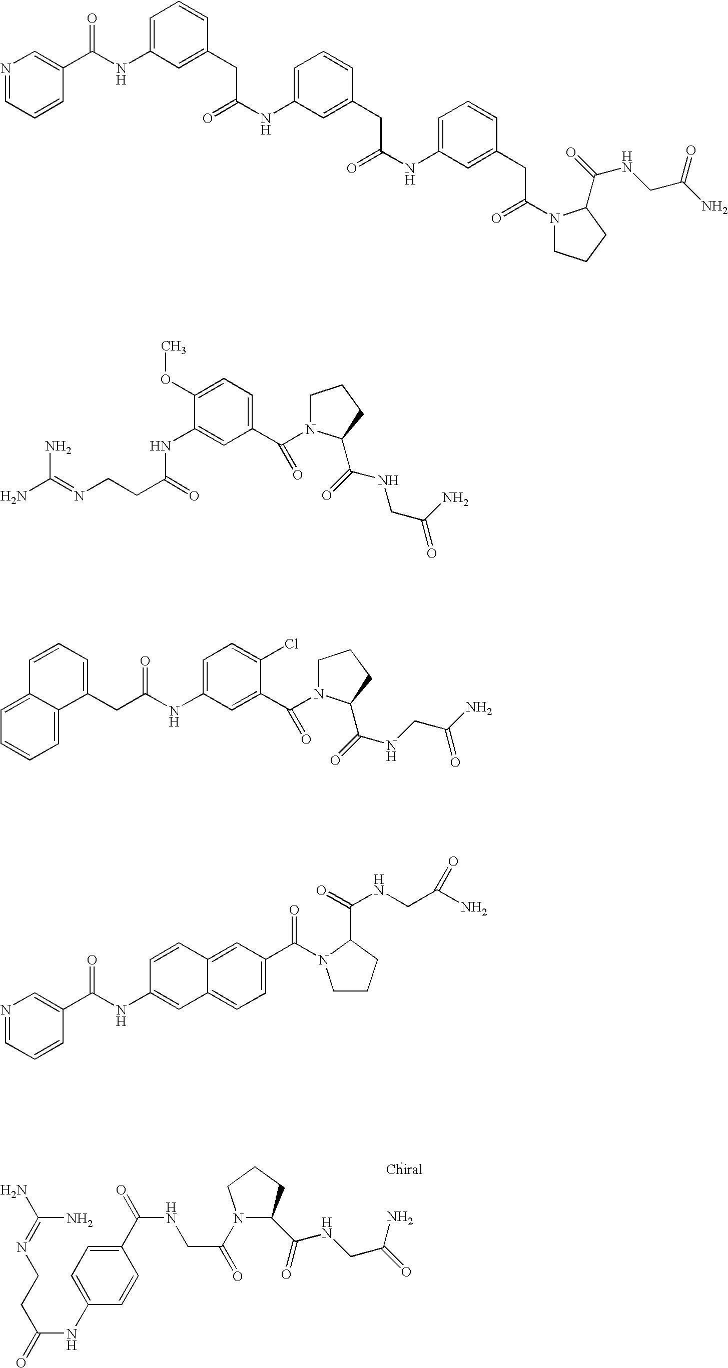

18. Compounds according to claim 1, in which AA₁ is an arginomimetic, AA₂-AA₃-AA₄ are substituted with a spacer, AA₅-AA₆ are substituted with a β-turn mimetic and AA₇ is an amino acid.

19. Compounds according to claim 18 with the formula

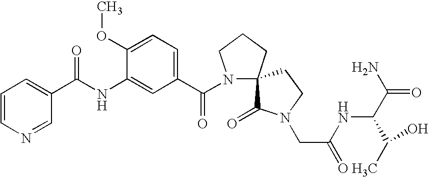

20. Compounds according to claim 1, in which AA₂-AA₃-AA₄ are substituted with a spacer.

21. Compounds according to claim 20 with the formula

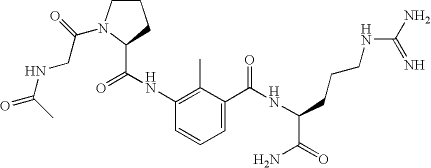

22. Compounds according to claim 1, in which one or more amino acids are substituted with one or more aza-aminoacids.

23. Compounds according to claim 16, selected from the group consisting of:
H-Arg-Gly-AzaVal-Val-Pro-Gly-NH₂
Ac-Azagly-Azagly-pro-leu-val-asp-arg-NH₂
Ac-Arg-Asp-Azagly-Val-Pro-Gly-NH₂
Ac-thr-Azagly-pro-leu-val-asp-arg-NH₂
Ac-Arg-Asp-Val-AzaVal-Pro-Gly-NH₂
Ac-Arg-Asp-AzaLeu-Val-Pro-Gly-NH₂

24. Use of compounds according to claim 1 as mimetics of a particular protein portion of MyD88, that prevent the homo-dimerisation of the protein by interfering with its interaction with the TIR domain.

25. Compounds according to claim 1 as medicaments.

26. Use of the compounds according to claim 1 for the preparation of a medicament useful for the treatment of diseases deriving from dysregulation of the signalling system of the TLR/IL-R1 receptor system.

27. Use according to claim 26, in which said diseases are selected from the group consisting of inflammatory and autoimmune diseases, cardiovascular and atherogenic diseases, sepsis and shock, transplant rejection, cancer and viral infections.

28. Use according to claim 27, in which said inflammatory and autoimmune diseases are selected from the group consisting of arthritis, gouty arthritis, chronic inflammatory bowel disease (IBD), psoriasis, type 1 diabetes, multiple sclerosis, asthma and systemic lupus erythematosus.

29. Use according to claim 27, in which said cardiovascular and atherogenic diseases are selected from the group consisting of myocardial infarct, viral myocarditis, atherosclerosis, vein graft atherosclerosis, thrombosis, re-stenosis, re-stenosis due to stents and re-stenosis due to angioplasty.

30. Pharmaceutical compositions containing at least one compound according to claim 1 in mixtures with at least one pharmaceutically acceptable vehicle and/or excipient.
